(19) United States
(12) Patent Application Publication
(10) Pub. No.: US 2026/0286386 A1
(43) Pub. Date: Sep. 24, 2026

(54) COMPOSITIONS AND METHODS FOR RECOMBINANT AAV PRODUCTION

(71) Applicant: REGENXBIO Inc., Rockville, MD (US)

(72) Inventors: Ping LIU, Rockville, MD (US); Ayda MAYER, Rockville, MD (US); Chih-Li LIN, Rockville, MD (US); Thilo H. POHLE, Sheffield (GB); Yusuf B. JOHARI, Sheffield (GB); David C. JAMES, Sheffield (GB)

(21) Appl. No.: 19/472,847

(22) PCT Filed: Apr. 5, 2024
(86) PCT No.: PCT/US2024/023368

§ 371 Date: Oct. 6, 2025

Related U.S. Application Data
(60) Provisional application No. 63/494,859, filed on Apr. 7, 2023.

(51) Int. Cl. C12N 15/86

(52) U.S. Cl. CPC C12N 15/86; C12N 2710/10322; C12N 2750/14143; C12N 2750/14152

(57) ABSTRACT

Provided herein are recombinant polynucleotides and plasmids suitable for use in the production of recombinant AAV particles. Also provided herein are methods for producing rAAV particles.

### CROSS-REFERENCE TO RELATED APPLICATIONS

This application claims the benefit of U.S. application No. 63/494,859, filed Apr. 7, 2023, which is incorporated herein by reference in its entirety.

### TECHNICAL FIELD

The present disclosure relates to recombinant polynucleotides and their use in a method of producing recombinant adeno-associated virus (rAAV) particles.

### REFERENCE TO SEQUENCE LISTING SUBMITTED ELECTRONICALLY

The content of the electronically submitted sequence listing (Name: 6728-2001_Sequence_Listing.xml; Size: 2,211,668 bytes; and Date of Creation: Apr. 4, 2024) filed with the application is incorporated herein by reference in its entirety.

### BACKGROUND

Recombinant adeno-associated virus (AAV)-based vectors are currently the most widely used gene therapy products in development. The preferred use of rAAV vector systems is due, in part, to the lack of disease associated with the wild-type virus, the ability of AAV to transduce non-dividing as well as dividing cells, and the resulting long-term robust transgene expression observed in clinical trials and that indicate great potential for delivery in gene therapy indications. Additionally, different naturally occurring and recombinant rAAV vector serotypes, specifically target different tissues, organs, and cells, and help evade any pre-existing immunity to the vector, thus expanding the therapeutic applications of AAV-based gene therapies. Before gene therapies based on a replication defective virus, for example, AAV can be more widely adopted for late clinical stage and commercial use, new methods for large scale production of recombinant virus particles need to be developed.

The triple plasmid transfection system in HEK293 cells is well established and commonly used for clinical and commercial manufacturing. In this system, one plasmid, often referred to as the trans plasmid, carries Rep and Cap genes and encodes proteins for virus replication and capsid formation. A second plasmid, often referred to as the helper plasmid, encodes the essential adenovirus helper genes (E4, E2A, and viral associated (VA) RNAs), and a third plasmid, often referred to as the cis plasmid, contains an expression cassette flanked by two inverted terminal repeats (ITRs), which is incorporated into the rAAV as its genome. The additional helper genes E1A and E1B are expressed endogenously by the HEK293 cells. The E1A protein increases Rep protein expression by transactivating the P5 and P19 promoters, and the Rep protein initiates AAV replication.

Reducing the number of plasmids for transient transfection from three to two or one can improve the transfection efficiency, simplify the process of transient transfection, lower the cost of goods (COGs) to manufacture GMP plasmids, and further shorten the timeline for producing a new rAAV. The triple plasmid system provided several advantages over the use of a helper virus to produce rAAV particles. These included short lead time for GMP plasmid manufacture, high speed of implantation to the clinic, and free of adenovirus in the process. The transfection efficiency for each plasmid, however, remains a limiting factor because the triple plasmid system requires cells to uptake three plasmids at the same time to produce viruses. Several research groups have tried to create a two-plasmid system to simplify the process of transient transfection, lower the cost of goods (COGs) to manufacture GMP plasmids, and further shorten the timeline for producing a new rAAV. The first two-plasmid expression system was developed by Grimm et al, in which they designed a packaging/helper plasmid pDG to include all genes from the trans plasmid and the helper plasmid into a single plasmid. In this new plasmid, the AAV-2 rep and cap, and adenovirus E4, E2A and VA RNA genes that are required for AAV replication, capsid formation and helper functions were included. Furthermore, the p5 promoter, which drove Rep expression in the trans plasmid, was replaced by a Mouse Mammary Tumor Virus (MMTV) promoter to weaken Rep protein expression. This new system showed a 10-fold higher titer than the conventional methods using a helper virus. Grimm et al., Novel tools for production and purification of recombinant adeno-associated virus vectors. Hum Gene Ther. 1998; 9(18):2745-2760. Tang et al. made further modifications of the backbone of pDG plasmid to create a pQT packaging system and included the rep, cap and the essential adenovirus helper genes needed for AAV production. They compared the pQT system with the conventional triple transfection system using different AAV serotypes including AAV1, AAV5 and AAV8 and AAV9 and found that the pQT system gave higher yield than the triple transfection system for AAV1 and AAV5 production. Tang et al., “Two-Plasmid Packaging System for Recombinant Adeno-Associated Virus.” Biores Open Access. 9 (2020): 219-28.

Because the pDG and pQT systems were designed by combining all the genes from trans and helper plasmids, the size of the plasmids is more than 20 Kb, which makes manufacturing of the plasmids very challenging. In addition to that, the large size of plasmids would lower the transfection efficiency during transient transfection. Thus, there is a need in the art to improve the productivity and yield of methods for the large-scale production of rAAV particles by providing improved helper and packaging plasmids.

### BRIEF SUMMARY

In one aspect, the disclosure provides an isolated recombinant polynucleotide comprising a) a first nucleotide sequence encoding an adenovirus E2A DNA binding protein (DBP) operably linked to a first promoter; b) a second nucleotide sequence encoding an adenovirus E4 polypeptide operably linked to a second promoter; c) a third nucleotide sequence encoding an adenovirus VA RNA I; and d) a fourth nucleotide sequence encoding a parvovirus p5 promoter and a fifth nucleotide sequence encoding an AAV rep gene and an AAV cap gene, wherein the parvovirus p5 promoter is operably linked to the AAV rep gene and controls the expression of the rep78 and rep68 gene products, optionally wherein the isolated recombinant polynucleotide does not comprise a nucleotide sequence encoding an adenovirus ITR sequence, L3 23K endoprotease, L5 pVI/fibre, and/or L4 pVIII/hexon-associated precursor. In some embodiments, the isolated recombinant polynucleotide further comprises a sixth nucleotide sequence encoding a recombinant viral genome comprising at least one AAV inverted terminal repeat (ITR) and a non-AAV nucleic acid sequence encoding a gene product operably linked to sequences which direct expression of the gene product in a target cell.

In one aspect, the disclosure provides a host cell comprising an isolated recombinant polynucleotide described herein.

In one aspect, the disclosure provides a method of producing an isolated recombinant polynucleotide described herein comprising incubating under suitable conditions a host cell described herein.

In one aspect, the disclosure provides a method of producing recombinant adeno-associated virus (rAAV) particles comprising culturing a cell capable of producing the rAAV particles under conditions that allow production of the rAAV particles, wherein the cell comprises a recombinant polynucleotide described herein.

In one aspect, the disclosure provides a method of producing rAAV particles, comprising a) providing a cell culture comprising a cell; b) introducing into the cell a recombinant polynucleotide described herein, and c) maintaining the cell culture under conditions that allow production of the rAAV particles.

In some embodiments, the disclosure provides:
[1.] An isolated recombinant polynucleotide comprising
   a) a first nucleotide sequence encoding an adenovirus E2A DNA binding protein (DBP) operably linked to a first promoter;
   b) a second nucleotide sequence encoding an adenovirus E4 polypeptide operably linked to a second promoter;
   c) a third nucleotide sequence encoding an adenovirus VA RNA I; and
   d) a fourth nucleotide sequence encoding a parvovirus p5 promoter and a fifth nucleotide sequence encoding an AAV rep gene and an AAV cap gene, wherein the parvovirus p5 promoter is operably linked to the AAV rep gene and controls the expression of the rep78 and rep68 gene products, optionally wherein the isolated recombinant polynucleotide does not comprise a nucleotide sequence encoding an adenovirus ITR sequence, L3 23K endoprotease, L5 pVI/fibre, and/or L4 pVIII/hexon-associated precursor;
[2.] the isolated recombinant polynucleotide of [1], wherein the nucleotide sequence encoding the adenovirus E2A DBP and the nucleotide sequence encoding the adenovirus E4 polypeptide are in opposite 5′ to 3′ orientation;
[3.] the isolated recombinant polynucleotide of [1] or [2], wherein the nucleotide sequence encoding the adenovirus E2A DBP has at least 80%, at least 85%, at least 90%, at least 95%, at least 97%, at least 98%, at least 99% or 100% identity to SEQ ID NO: 1 or 60;
[4.] the isolated recombinant polynucleotide of any one of [1] to [3], wherein the adenovirus E2A DBP comprises an amino acid sequence having at least 80%, at least 85%, at least 90%, at least 95%, at least 97%, at least 98%, at least 99% or 100% identity to SEQ ID NO: 45;
[5.] the isolated recombinant polynucleotide of any one of [1] to [4], wherein the E4 polypeptide comprises the E4 ORF6 and ORF7;
[6.] the isolated recombinant polynucleotide of [5], wherein the nucleotide sequence encoding the adenovirus E4 ORF6 and ORF7 has at least 80%, at least 85%, at least 90%, at least 95%, at least 97%, at least 98%, at least 99% or 100% identity to SEQ ID NO: 8 or 61;
[7.] the isolated recombinant polynucleotide of [5] or [6], wherein the adenovirus E4 ORF6 polypeptide comprises an amino acid sequence having at least 80%, at least 85%, at least 90%, at least 95%, at least 97%, at least 98%, at least 99% or 100% identity to SEQ ID NO: 46 and the adenovirus E4 ORF7 polypeptide comprises an amino acid sequence having at least 80%, at least 85%, at least 90%, at least 95%, at least 97%, at least 98%, at least 99% or 100% identity to SEQ ID NO: 120;
[8.] the isolated recombinant polynucleotide of any one of [1] to [4], wherein the E4 polypeptide comprises the E4 ORF6;
[9.] the isolated recombinant polynucleotide of [8], wherein the nucleotide sequence encoding the adenovirus E4 ORF6 has at least 80%, at least 85%, at least 90%, at least 95%, at least 97%, at least 98%, at least 99% or 100% identity to SEQ ID NO: 6;
[10.] the isolated recombinant polynucleotide of [9] or [10], wherein the adenovirus E4 ORF6 polypeptide comprises an amino acid sequence having at least 80%, at least 85%, at least 90%, at least 95%, at least 97%, at least 98%, at least 99% or 100% identity to SEQ ID NO: 46;
[11.] the isolated recombinant polynucleotide of any one of [1] to [10], wherein the nucleotide sequence encoding the adenovirus VA RNA I comprises a nucleotide sequence having at least 80%, at least 85%, at least 90%, at least 95%, at least 97%, at least 98%, at least 99% or 100% identity to SEQ ID NO: 54;
[12.] the isolated recombinant polynucleotide of any one of [1] to [10], wherein the nucleotide sequence encoding the adenovirus VA RNA I encodes VA RNA I and VARNA II, and optionally comprises a nucleotide sequence having at least 80%, at least 85%, at least 90%, at least 95%, at least 97%, at least 98%, at least 99% or 100% identity to SEQ ID NO: 9;
[13.] the isolated recombinant polynucleotide of any one of [1] to [12], wherein the first promoter and second promoter are different promoters;
[14.] the isolated recombinant polynucleotide of any one of [1] to [13], wherein the first promoter is an adenovirus E2A promoter;
[15.] the isolated recombinant polynucleotide of [14], wherein the adenovirus E2A promoter comprises a nucleotide sequence having at least 80%, at least 85%, at least 90%, at least 95%, at least 97%, at least 98%, at least 99% or 100% identity to SEQ ID NO: 2;
[16.] the isolated recombinant polynucleotide of any one of [1] to [15], wherein the first nucleotide sequence comprises a nucleotide sequence having at least 80%, at least 85%, at least 90%, at least 95%, at least 97%, at least 98%, at least 99% or 100% identity to SEQ ID NO: 3, 4, 23, 24, 273-275 or 276;
[17.] the isolated recombinant polynucleotide of any one of [1] to [16], wherein the second promoter is an adenovirus E4 promoter;
[18.] the isolated recombinant polynucleotide of [17], wherein the adenovirus E4 promoter comprises a nucleotide sequence having at least 80%, at least 85%, at least 90%, at least 95%, at least 97%, at least 98%, at least 99% or 100% identity to SEQ ID NO: 5;
[19.] the isolated recombinant polynucleotide of any one of [1] to comprising a nucleotide sequence having at least 80%, at least 85%, at least 90%, at least 95%, at least 97%, at least 98%, at least 99% or 100% identity to SEQ ID NO: 10 or 11;
[20.] the isolated recombinant polynucleotide of any one of [1] to [18], wherein the isolated recombinant polynucleotide comprises a nucleotide sequence encoding the E2A promoter, L4 22K/33K polypeptides and promoter, L4 100 k/hexon assembly polypeptide comprising an N terminal deletion and the E2A DBP, wherein the N-terminal deletion of the L4 100 k/hexon assembly polypeptide corresponds to the nucleotide sequence of SEQ ID NO: 21;
[21.] the isolated recombinant polynucleotide of [20], wherein the nucleotide sequence encoding the E2A promoter, L4 22K/33K polypeptides and promoter, L4 100 k/hexon assembly polypeptide comprising an N terminal deletion and the E2A DBP comprises a nucleotide sequence having at least 80%, at least 85%, at least 90%, at least 95%, at least 97%, at least 98%, at least 99% or 100% identity to SEQ ID NO: 22;
[22.] the isolated recombinant polynucleotide of any one of [1] to [18], wherein the isolated recombinant polynucleotide comprises a nucleotide sequence encoding the E2A promoter, L4 22K/33K polypeptides and promoter, L4 100 k/hexon assembly polypeptide comprising a mutation in its start codon and the E2A DBP;
[23.] the isolated recombinant polynucleotide of [22], wherein the nucleotide sequence encoding the E2A promoter, L4 22K/33K polypeptides and promoter, L4 100 k/hexon assembly polypeptide comprising a mutation in its start codon and the E2A DBP comprises a nucleotide sequence having at least 80%, at least 85%, at least 90%, at least 95%, at least 97%, at least 98%, at least 99% or 100% identity to SEQ ID NO: 23;
[24.] the isolated recombinant polynucleotide of any one of [1] to [18], wherein the isolated recombinant polynucleotide comprises a nucleotide sequence encoding the E2A promoter, L4 22K/33K polypeptides and promoter, L4 100 k/hexon assembly polypeptide comprising an N terminal deletion and the E2A DBP, wherein the N-terminal deletion of the L4 100 k/hexon assembly polypeptide encompasses the start codon of L4 100 k/hexon assembly but does not encompass the start codon of the L4 22K/33K polypeptides;
[25.] the isolated recombinant polynucleotide of any one of [1] to [18], wherein the isolated recombinant polynucleotide comprises a nucleotide sequence encoding the E2A promoter, L4 22K/33K polypeptides and promoter, L4 100 k/hexon assembly polypeptide comprising an N terminal deletion and the E2A DBP, wherein all or part of the L4 100 k/hexon assembly polypeptide is deleted without disruption of the L4 22K/33K start codon;
[26.] the isolated recombinant polynucleotide of any one of [1] to [18], wherein the isolated recombinant polynucleotide comprises a nucleotide sequence encoding the E2A promoter, L4 22K/33K polypeptides and promoter, L4 100 k/hexon assembly polypeptide comprising an N terminal deletion and the E2A DBP, wherein the N-terminal deletion of the L4 100 k/hexon assembly starts at the start codon of L4 100 k/hexon assembly and ends immediately adjacent to the L4 22K/33K promoter;
[27.] the isolated recombinant polynucleotide of any one of [1] to comprising a nucleotide sequence having at least 80%, at least 85%, at least 90%, at least 95%, at least 97%, at least 98%, at least 99% or 100% identity to SEQ ID NO: 25-34, 56, 57, 106-109, 122-130 or 131;
[28.] the isolated recombinant polynucleotide of any one of [1] to comprising a nucleotide sequence having at least 80%, at least 85%, at least 90%, at least 95%, at least 97%, at least 98%, at least 99% or 100% identity to SEQ ID NO: 140-158, 165, 170, 175, 180, 185, 190, 195, 200, 205, 210, 215, 220, 225, 230, 235, 240, 245, 250, 255, or 260;
[29.] the isolated recombinant polynucleotide of any one of [1] to comprising a nucleotide sequence having at least 80%, at least 85%, at least 90%, at least 95%, at least 97%, at least 98%, at least 99% or 100% identity to SEQ ID NO: 265 or 266;
[30.] the isolated recombinant polynucleotide of any one of [1] to [29], wherein the parvovirus p5 promoter is an AAV p5 promoter;
[31.] the isolated recombinant polynucleotide of [30], AAV p5 promoter comprises a nucleotide sequence having at least 80%, at least 85%, at least 90%, at least 95%, at least 97%, at least 98%, at least 99% or 100% identity to SEQ ID NO: 62;
[32.] the isolated recombinant polynucleotide of any one of [1] to [31], wherein the AAV rep gene and the AAV cap gene have the same serotype;
[33.] the isolated recombinant polynucleotide of any one of [1] to [31], wherein the AAV rep gene and the AAV cap gene have different serotypes;
[34.] the isolated recombinant polynucleotide of any one of [1] to [31], wherein the AAV rep gene comprises an AAV2 rep gene;
[35.] the isolated recombinant polynucleotide of [34], wherein the AAV rep gene comprises a nucleotide sequence having at least 80%, at least 85%, at least 90%, at least 95%, at least 97%, at least 98%, at least 99% or 100% identity to SEQ ID NO: 63;
[36.] the isolated recombinant polynucleotide of any one of [1] to [35], wherein the AAV cap gene comprises a serotype selected from the group consisting of AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16;
[37.] the isolated recombinant polynucleotide of [36], wherein the AAV cap gene comprises a serotype selected from the group consisting of AAV8, AAV9, AAV.rh10, AAV.rh20, AAV.rh39, AAV.Rh74, AAV.RHM4-1, AAV.hu32, and AAV.hu37;
[38.] the isolated recombinant polynucleotide of [36], wherein the AAV cap gene comprises a serotype selected from the group consisting of AAV8 or AAV9 serotype;
[39.] the isolated recombinant polynucleotide of [36], wherein the AAV cap gene comprises the AAV2 scrotype;
[40.] the isolated recombinant polynucleotide of [39], wherein the AAV2 cap gene comprises a nucleotide sequence having at least 80%, at least 85%, at least 90%, at least 95%, at least 97%, at least 98%, at least 99% or 100% identity to SEQ ID NO: 64;
[41.] the isolated recombinant polynucleotide of [36], wherein the AAV cap gene comprises the AAV6 serotype;
[42.] the isolated recombinant polynucleotide of [41], wherein the AAV6 cap gene comprises a nucleotide sequence having at least 80%, at least 85%, at least 90%, at least 95%, at least 97%, at least 98%, at least 99% or 100% identity to SEQ ID NO: 65;
[43.] the isolated recombinant polynucleotide of [36], wherein the AAV cap gene comprises the AAV8 serotype;
[44.] the isolated recombinant polynucleotide of [43], wherein the AAV8 cap gene comprises a nucleotide sequence having at least 80%, at least 85%, at least 90%, at least 95%, at least 97%, at least 98%, at least 99% or 100% identity to SEQ ID NO: 66;
[45.] the isolated recombinant polynucleotide of [36], wherein the AAV cap gene comprises the AAV9 serotype;
[46.] the isolated recombinant polynucleotide of [45], wherein the AAV9 cap gene comprises a nucleotide sequence having at least 80%, at least 85%, at least 90%, at least 95%, at least 97%, at least 98%, at least 99% or 100% identity to SEQ ID NO: 67;
[47.] the isolated recombinant polynucleotide of any one of [1] to [46], wherein the sequence encoding the cap gene comprises one or more mutations that disrupt the expression of the mAAP polypeptide;
[48.] the isolated recombinant polynucleotide of [47], wherein the one or mutations that disrupt the expression of the mAAP polypeptide comprise one or more non-sense mutations in the mAAP ORF;
[49.] the isolated recombinant polynucleotide of [47], wherein the one or mutations that disrupt the expression of the mAAP polypeptide comprise a mutation in the start codon of the mAAP ORF;
[50.] the isolated recombinant polynucleotide of any one of [1] to [46], wherein the sequence encoding the cap gene comprises the nucleotide sequence of SEQ ID NO: 166, 171, 176, 181, 186, 191, 196, 201, 206, 211, 216, 221, 226, 231, 236, 241, 246, 251, 256, or 261;
[51.] the isolated recombinant polynucleotide of any one of [1] to [50], wherein the fifth nucleotide sequence has at least 80%, at least 85%, at least 90%, at least 95%, at least 97%, at least 98%, at least 99% or 100% identity to SEQ ID NO: 68-70 or 71;
[52.] the isolated recombinant polynucleotide of any one of [1] to [50], wherein the fifth nucleotide sequence has at least 80%, at least 85%, at least 90%, at least 95%, at least 97%, at least 98%, at least 99% or 100% identity to SEQ ID NO: 167, 172, 187, 192, 197, 202, 207, 212, 217, 222, 227, 232, 237, 242, 247, 252, 257, or 262;
[53.] the isolated recombinant polynucleotide of any one of [1] to [52], wherein the p5 promoter is positioned between about 10 and about 10,000 nucleotides upstream from the AAV rep start codon;
[54.] the isolated recombinant polynucleotide of any one of [1] to [52], wherein the p5 promoter is positioned between about 5,000 and about 10,000 nucleotides upstream from the AAV rep start codon;
[55.] the isolated recombinant polynucleotide of any one of [1] to [52], wherein the p5 promoter is positioned between about 1,000 and about 5,000 nucleotides upstream from the AAV rcp start codon;
[56.] the isolated recombinant polynucleotide of [53], wherein the p5 promoter is positioned between about 1,000 and about 4,000 nucleotides upstream from the AAV rep start codon;
[57.] the isolated recombinant polynucleotide of [53], wherein the p5 promoter is positioned between about 1,000 and about 3,000 nucleotides upstream from the AAV rep start codon;
[58.] the isolated recombinant polynucleotide of [53], wherein the p5 promoter is positioned between about 2,000 and about 5,000 nucleotides upstream from the AAV rep start codon;
[59.] the isolated recombinant polynucleotide of [53], wherein the p5 promoter is positioned between about 2,000 and about 4,000 nucleotides upstream from the AAV rep start codon;
[60.] the isolated recombinant polynucleotide of [53], wherein the p5 promoter is positioned between about 2,000 and about 3,000 nucleotides upstream from the AAV rep start codon;
[61.] the isolated recombinant polynucleotide of any one of [1] to [52], wherein the first, second and/or third nucleotide sequence is positioned between the p5 promoter and the AAV rep start codon upstream from the AAV rep start codon;
[62.] the isolated recombinant polynucleotide of any one of [1] to [52], wherein the first, second and third nucleotide sequences are positioned between the p5 promoter and the AAV rep start codon upstream from the AAV rep start codon;
[63.] the isolated recombinant polynucleotide of any one of [1] to [52], wherein the first, second or third nucleotide sequence is not positioned between the p5 promoter and the AAV rep start codon upstream from the AAV rep start codon;
[64.] the isolated recombinant polynucleotide of any one of [1] to comprising a nucleotide sequence having at least 80%, at least 85%, at least 90%, at least 95%, at least 97%, at least 98%, at least 99% or 100% identity to SEQ ID NO: 72-86, 118, 159-161, 168, 173, 178, 183, 188, 193, 198, 203, 208, 213, 218, 223, 228, 233, 238, 243, 248, 253, 558, or 263;
[65.] the isolated recombinant polynucleotide of any one of [1] to comprising a nucleotide sequence having at least 80%, at least 85%, at least 90%, at least 95%, at least 97%, at least 98%, at least 99% or 100% identity to SEQ ID NO: 76 or 77;
[66.] the isolated recombinant polynucleotide of any one of [1] to comprising a nucleotide sequence having at least 80%, at least 85%, at least 90%, at least 95%, at least 97%, at least 98%, at least 99% or 100% identity to SEQ ID NO: 87-101, 119, 162-164, 169, 174, 179, 184, 189, 194, 199, 204, 209, 214, 219, 224, 229, 234, 239, 244, 249, 254, 259, or 264;
[67.] the isolated recombinant polynucleotide of any one of [1] to comprising a nucleotide sequence having at least 80%, at least 85%, at least 90%, at least 95%, at least 97%, at least 98%, at least 99% or 100% identity to SEQ ID NO: 91 or 92;
[68.] the isolated recombinant polynucleotide of any one of [1] to [67], wherein the isolated recombinant polynucleotide is a plasmid comprising a bacterial replication origin and a selectable marker gene;
[69.] the isolated recombinant polynucleotide of [68], wherein the bacterial replication origin is a ColE1 origin;
[70.] the isolated recombinant polynucleotide of or [56], wherein the selectable marker gene is a drug resistance gene;
[71.] the isolated recombinant polynucleotide of [70], wherein the selectable marker gene is a kanamycin resistance gene;
[72.] the isolated recombinant polynucleotide of any one of [1] to [71], wherein the bacterial replication origin or the selectable marker gene are positioned between the fourth nucleotide and the fifth nucleotide such that transcription initiated at the p5 promoter traverses the bacterial replication origin or the selectable marker gene, respectively, before reaching the AAV rep gene;
[73.] the isolated recombinant polynucleotide of any one of [1] to [71], wherein the bacterial replication origin and the selectable marker gene are positioned between the fourth nucleotide and the fifth nucleotide such that transcription initiated at the p5 promoter traverses the bacterial replication origin and the selectable marker gene before reaching the AAV rep gene;
[74.] the isolated recombinant polynucleotide of any one of [1] to comprising a sixth nucleotide sequence encoding a recombinant viral genome comprising at least one AAV inverted terminal repeat (ITR) and a non-AAV nucleic acid sequence encoding a gene product operably linked to sequences which direct expression of the gene product in a target cell;
[75.] the isolated recombinant polynucleotide of [74], wherein the AAV rep gene, AAV cap gene and non-AAV nucleic acid sequence encoding the gene product are transcribed in the same direction;
[76.] the isolated recombinant polynucleotide of comprising a nucleotide sequence having at least 80%, at least 85%, at least 90%, at least 95%, at least 97%, at least 98%, at least 99% or 100% identity to SEQ ID NO: 102, 103, 267 or 268 excluding the nucleotide residues corresponding to the nucleotide sequence encoding the recombinant viral genome;
[77.] the isolated recombinant polynucleotide of comprising a nucleotide sequence having at least 80%, at least 85%, at least 90%, at least 95%, at least 97%, at least 98%, at least 99% or 100% identity to SEQ ID NO: 104 or 105 excluding residues 9,537-14,270 of SEQ ID NO: 104 and residues 9,526-14,259 of SEQ ID NO: 105 corresponding to the nucleotide sequence encoding the recombinant viral genome;
[78.] the isolated recombinant polynucleotide of any one of [1] to [77], which does not comprise a nucleotide sequence encoding an adenovirus ITR sequence, L3 23K endoprotease, L5 pVI/fibre, or L4 pVIII/hexon-associated precursor.
[79.] A host cell comprising the isolated recombinant polynucleotide of any one of [1] to [78];
[80.] the host cell of [79], wherein the host cell is a bacterial cell;
[81.] the host cell of [79], wherein the host cell is an E. coli cell;
[82.] the host cell of [79], wherein the host cell is a eukaryotic cell;
[83.] the host cell of [79], wherein the host cell is a mammalian cell;
[84.] the host cell of [79], wherein the host cell is a HEK293 cell, HEK derived cell, CHO cell, CHO derived cell, HeLa cell, SF-9 cell, BHK cell, Vero cell, or PerC6 cell.
[85.] A method of producing the isolated recombinant polynucleotide of any one of [1] to [78] comprising incubating under suitable conditions the host cell of any of [79] to [84];
[86.] the method of comprising incubating under suitable conditions the host cell of or [81].
[87.] A method of producing rAAV particles, comprising
   a) providing a cell culture comprising a cell;
   b) introducing into the cell
      i. a polynucleotide of any one of [1] to [73]; and
      ii. a polynucleotide comprising a genome comprising at least one AAV inverted terminal repeat (ITR) and a non-AAV nucleic acid sequence encoding a gene product operably linked to sequences which direct expression of the gene product in a target cell, and
   c) maintaining the cell culture under conditions that allow production of the rAAV particles.
[88.] A method of producing rAAV particles, comprising
   a) providing a cell culture comprising a cell;
   b) introducing into the cell a polynucleotide of any one of to [77]; and
   c) maintaining the cell culture under conditions that allow production of the rAAV particles;
[89.] the method of or [88], wherein the introducing of the polynucleotides or polynucleotide into the cell is by transfection;
[90.] the method of any one of to [89], wherein the cell is a mammalian cell;
[91.] the method of any one of to [89], wherein the cell is an insect cell;
[92.] the method of any one of to [89], wherein the cell is a HEK293 cell, HEK derived cell, CHO cell, CHO derived cell, HeLa cell, SF-9 cell, BHK cell, Vero cell, or PerC6 cell;
[93.] the method of any one of to [89], wherein the cell is a HEK293 cell;
[94.] the method of any one of to [93], wherein the cell culture is a suspension culture or an adherent culture;
[95.] the method of any one of to [94, further comprising recovering the rAAV particles;
[96.] the method of any one of to [95], wherein the method produces more rAAV particles measured as GC/ml than a reference method using a polynucleotide comprising helper functions comprising the nucleotide sequence of SEQ ID NO: 44;
[97.] the method of any one of to [95], wherein the method produces at least about twice as many rAAV particles measured as GC/ml than a reference method using a polynucleotide comprising helper functions comprising the nucleotide sequence of SEQ ID NO: 44;
[98.] the method of any one of to [95], wherein the method produces a population of rAAV particles comprising more full capsids than a reference method using a polynucleotide comprising helper functions comprising the nucleotide sequence of SEQ ID NO: 44;
[99.] the method of any one of to [98], wherein the cell culture has a volume between about 50 liters and about 20,000 liters;
[100.] the method of any one of to [99], wherein the gene product is a polypeptide or a double stranded RNA molecule;
[101.] the method of [100], wherein the gene product is a polypeptide;
[102.] the method of [101], wherein the gene product is anti-VEGF Fab, anti-kallikrein antibody, anti-TNF antibody, microdystrophin, minidystrophin, iduronidase (IDUA), iduronate 2-sulfatase (IDS), low-density lipoprotein receptor (LDLR), tripeptidyl peptidase 1 (TPP1), or non-membrane associated splice variant of VEGF receptor 1 (sFlt-1);
[103.] the method of [101], wherein the gene product is an gamma-sarcoglycan, Rab Escort Protein 1 (REP1/CHM), retinoid isomerohydrolase (RPE65), cyclic nucleotide gated channel alpha 3 (CNGA3), cyclic nucleotide gated channel beta 3 (CNGB3), aromatic L-amino acid decarboxylase (AADC), lysosome-associated membrane protein 2 isoform B (LAMP2B), Factor VIII, Factor IX, retinitis pigmentosa GTPase regulator (RPGR), retinoschisin (RS1), sarcoplasmic reticulum calcium ATPase (SERCA2a), aflibercept, battenin (CLN3), transmembrane ER protein (CLN6), glutamic acid decarboxylase (GAD), Glial cell line-derived neurotrophic factor (GDNF), aquaporin 1 (AQP1), dystrophin, myotubularin 1 (MTM1), follistatin (FST), glucose-6-phosphatase (G6Pase), apolipoprotein A2 (APOA2), uridine diphosphate glucuronosyl transferase 1A1 (UGT1A1), arylsulfatase B (ARSB), N-acetyl-alpha-glucosaminidase (NAGLU), alpha-glucosidase (GAA), alpha-galactosidasc (GLA), beta-galactosidasc (GLB1), lipoprotein lipase (LPL), alpha 1-antitrypsin (AAT), phosphodiesterase 6B (PDE6B), ornithine carbamoyltransferase 90TC), survival motor neuron (SMN1), survival motor neuron (SMN2), neurturin (NRTN), Neurotrophin-3 (NT-3/NTF3), porphobilinogen deaminase (PBGD), nerve growth factor (NGF), mitochondrially encoded NADH: ubiquinone oxidoreductase core subunit 4 (MT-ND4), protective protein cathepsin A (PPCA), dysferlin, MER proto-oncogene, tyrosine kinase (MERTK), cystic fibrosis transmembrane conductance regulator (CFTR), or tumor necrosis factor receptor (TNFR)-immunoglobulin (IgG1) Fc fusion;
[104.] the method of [101], wherein the gene product is a dystrophin or a microdystrophin;
[105.] the method of [100], wherein the gene product is a microRNA or an antisense RNA.

Still other features and advantages of the compositions and methods described herein will become more apparent from the following detailed description when read in conjunction with the accompanying drawings.

### BRIEF DESCRIPTION OF THE DRAWINGS

FIG. 1. pHRC plasmid #1 map.

FIG. 2. pHRC plasmid #2 map.

FIG. 3. pHRC plasmid #3 map.

FIG. 4. Production of TG-A rAAV8 particles using pHRC #1, pHRC #2 or pHRC #3 plasmid and cis TG-A plasmid.

FIG. 5. Production of TG-A rAAV8 particles using pHRC #3 plasmid and cis TG-A plasmid.

FIG. 6. Production of TG-B rAAV8 particles using pHRC #3 plasmid and cis TG-B plasmid. m1, m2 and m3 indicate different absolute mass of pHRC plasmid #3 used.

FIG. 7. pHRC plasmid #4 map.

FIG. 8. pHRC plasmid #5 map.

FIG. 9. Production of TG-A rAAV8 particles using pHRC #3, pHRC #4 or pHRC #5 plasmid and cis TG-A plasmid.

FIG. 10. Production of TG-A rAAV8 particles using pHRC #3 or pHRC #7 plasmid and cis TG-A plasmid.

FIG. 11. Production of TG-A rAAV8 particles using Helper #5 or Helper #10 plasmid.

FIG. 12. pHRC plasmid #7 map.

FIG. 13. Production of TG-A rAAV8 particles using pHRC #3, pHRC #5 or pHRC #7 plasmid.

FIG. 14. TG-A rAAV8 particle production using different expression systems at day 1, day 2 and day 3 after transfection. A: ddPCR titers, B: ELISA titers, and C: full % calculation based on ddPCR and ELISA titers.

FIG. 15. Western blot analysis to detect virus capsid proteins in TG-A rAAV8 particles. A: Western blot analysis of VP1, VP2 and VP3 capsid proteins. B: Comparison of the fold changes of total VP1/2/3 in two cell lines using different plasmids. C: Comparison of the fold changes of total VP1/2/3 in two cell lines using different plasmids. D: D: Fold-changes of VP1/2/3 normalized to vinculin in the cell line Clone 4-11A4.

FIG. 16. Western blot analysis to detect Replicase proteins in TG-A rAAV8 particles. A: Western blot analysis of Rep78, Rep52 and Rep40 proteins. B: Comparison of the fold changes of total Rep78/Rep52/Rep40 in two cell lines using different plasmids. C: Fold-changes of Rep78/Rep52/Rep40 normalized to vinculin in the cell line Clone 1. D: Fold-changes of Rep78/Rep52/Rep40 normalized to vinculin in the cell line Clone 4-11A4.

FIG. 17. Production of TG-B rAAV8 particles using Original helper, Helper #3, Helper #5, pHRC #3, pHRC #5, or pHRC #7 plasmid.

FIG. 18. Production of TG-C rAAV9 particles using Original helper, Helper #3, Helper #5, or pHRC #8 plasmid.

FIG. 19. Production of TG-D rAAV9 particles using Original helper, Helper #3, Helper #5, or pHRC #8 plasmid.

FIG. 20. Production of TG-A rAAV9 particles using Original helper, Helper #3, Helper #5, pHRC #8, pHRC #11, or pHRC #14 plasmid.

FIG. 21. Production of TG-D rAAV9 particles using Original helper, Helper #3, Helper #5, pHRC #8, pHRC #11, or pHRC #14 plasmid.

FIG. 22. Production of TG-A rAAV2 particles using Original helper, Helper #3, Helper #5, pHRC #9, pHRC #12, or pHRC #15 plasmid.

FIG. 23. Production of TG-A rAAV8 particles using Helper #5, pHRC #5, or pHRC #7 plasmid. A: ddPCR titers, B: % full calculation based on analytical ultracentrifugation (AUC).

FIG. 24. pHRC plasmid #35 map.

FIG. 25. pHRC plasmid #36 map.

FIG. 26. pHRC plasmid #37 map.

FIG. 27. pHRCG plasmid #1 map.

FIG. 28. Production of TG-A rAAV8 particles using Original helper, Helper #5, pHRC #7, or pHRCG #1 plasmid.

FIG. 29. pHRCG plasmid #2 map.

FIG. 30. Production of TG-A rAAV8 particles using Original helper, Helper #5, pHRC #7, pHRCG #1, or pHRCG #2 plasmid.

FIG. 31. Production of TG-A rAAV8 particles using Original helper, Helper #5, pHRC #7, or pHRCG #1 plasmid. A: Study 1. B: Study 2.

FIG. 32. pHRCG plasmid #3 map.

FIG. 33. pHRCG plasmid #4 map.

FIG. 34. Production of TG-A rAAV8 particles using Original helper, Helper #5, pHRC #7, pHRCG #1, pHRCG #3, or pHRCG #4 plasmid. A: ddPCR titers, B: ELISA titers, and C: full % calculation based on ddPCR and ELISA titers.

FIG. 35. Production of TG-A rAAV8 particles using Original helper, Helper #5, pHRC #3, pHRC #5, pHRC #7, or pHRCG #1 plasmid. A: ddPCR titers, B: ELISA titers, and C: full % calculation based on ddPCR and ELISA titers.

FIG. 36. Production of TG-A rAAV2 particles using Original helper, Helper #3, Helper #5, pHRC #15, pHRC #17, pHRC #18, pHRC #19, pHRC #20, pHRC #21, or pHRCG #22 plasmid.

FIG. 37. mAAP mutations impact rAAV8 virus production. Production of TG-A (A), TG-B (B) and TG-D (C) rAAV8 particles using Original helper, Helper #3, Helper #5, pHRC #7, pHRC #23, pHRC #24, pHRC #25, pHRC #26, pHRC #27, or pHRCG #28 plasmid.

FIG. 38. mAAP mutations impact rAAV9 virus production. Production of TG-A rAAV9 particles using Original helper, Helper #3, Helper #5, pHRC #8, pHRC #29, pHRC #30, pHRC #31, pHRC #32, pHRC #33, or pHRCG #34 plasmid.

FIG. 39. mAAP mutations impact rAAV9 virus production. Production of TG-D rAAV9 particles using Original helper, Helper #3, Helper #5, pHRC #8, pHRC #29, pHRC #30, pHRC #31, pHRC #32, pHRC #33, or pHRCG #34 plasmid.

FIG. 40. mAAP mutations impact rAAV8 virus secretion. Production of TG-A (A), TG-B (B) and TG-D (C) rAAV8 particles using Original helper, Helper #3, Helper #5, pHRC #7, pHRC #23, pHRC #24, pHRC #25, pHRC #26, pHRC #27, or pHRCG #28 plasmid.

FIG. 41. % full TG-A (A), TG-B (B) and TG-D (C) rAAV8 particles produced using Original helper, Helper #3, Helper #5, pHRC #7, pHRC #23, pHRC #24, pHRC #25, pHRC #26, pHRC #27, or pHRCG #28 plasmid. % full calculation based on ddPCR and ELISA titers.

FIG. 42. % full TG-A (A) and TG-D (B) rAAV9 particles produced using Original helper, Helper #3, Helper #5, pHRC #8, pHRC #29, pHRC #30, pHRC #31, pHRC #32, pHRC #33, or pHRCG #34 plasmid. % full calculation based on ddPCR and ELISA titers.

### DETAILED DESCRIPTION

In one aspect, provided herein are improved recombinant polynucleotides and plasmids encoding helper functions, an AAV rep gene and an AAV cap gene suitable for use in the production of recombinant AAV particles. In some embodiments, the helper functions comprise a nucleotide sequence encoding an adenovirus E2A DNA binding protein, a nucleotide sequence encoding an adenovirus E4 polypeptide and a nucleotide sequence encoding an adenovirus VA RNA I. In some embodiments, the adenovirus E4 polypeptide comprises the E4 ORF6 and ORF7 or the E4 ORF6. In some embodiments, the polynucleotides and plasmids do not comprise a nucleotide sequence encoding an adenovirus ITR sequence, L3 23K endoprotease, L5 pVI/fibre, and/or L4 pVIII/hexon-associated precursor. In some embodiments, the polynucleotides and plasmids are smaller than previously available polynucleotides and plasmids encoding helper functions and AAV rep/cap genes suitable for use in the production of recombinant AAV particles. In some embodiments, use of the improved polynucleotides and plasmids described herein in the production of recombinant AAV particles results in increased rAAV yield.

In one aspect, provided herein are improved recombinant polynucleotides and plasmids encoding helper functions, an AAV rep gene, an AAV cap gene and a recombinant AAV viral genome suitable for use in the production of recombinant AAV particles. In some embodiments, the recombinant AAV viral genome comprises at least one AAV inverted terminal repeat (ITR) and a non-AAV nucleic acid sequence encoding a gene product operably linked to sequences which direct expression of the gene product in a target cell. In some embodiments, the helper functions comprise a nucleotide sequence encoding an adenovirus E2A DNA binding protein, a nucleotide sequence encoding an adenovirus E4 polypeptide and a nucleotide sequence encoding an adenovirus VA RNA I. In some embodiments, the adenovirus E4 polypeptide comprises the E4 ORF6 and ORF7 or the E4 ORF6. In some embodiments, the polynucleotides and plasmids do not comprise a nucleotide sequence encoding an adenovirus ITR sequence, L3 23K endoprotease, L5 pVI/fibre, and/or L4 pVIII/hexon-associated precursor.

### Definitions

Unless defined otherwise, all technical and scientific terms used herein have the same meaning as commonly understood by one of ordinary skill in the art to which this disclosure is related. To facilitate an understanding of the disclosed methods, a number of terms and phrases are defined below.

“AAV” is an abbreviation for adeno-associated virus, and may be used to refer to the virus itself or modifications, derivatives, or pseudotypes thereof. The term covers all subtypes and both naturally occurring and recombinant forms, except where required otherwise. The abbreviation “rAAV” refers to recombinant adeno-associated virus. The term “AAV” includes AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), avian AAV, bovine AAV, canine AAV, equine AAV, primate AAV, non-primate AAV, and ovine AAV, and modifications, derivatives, or pseudotypes thereof. “Primate AAV” refers to AAV that infects primates, “non-primate AAV” refers to AAV that infects non-primate mammals, “bovine AAV” refers to AAV that infects bovine mammals, etc.

“Recombinant”, as applied to an AAV particle means that the AAV particle is the product of one or more procedures that result in an AAV particle construct that is distinct from an AAV particle in nature.

A recombinant adeno-associated virus particle “rAAV particle” refers to a viral particle composed of at least one AAV capsid protein and an encapsidated polynucleotide rAAV vector genome comprising a heterologous polynucleotide (i.e., a polynucleotide other than a wild-type AAV genome such as a transgene to be delivered to a mammalian cell). The rAAV particle may be of any AAV serotype, including any modification, derivative or pseudotype (e.g., AAV1, AAV2, AAV3, AAV4, AAV5, AAV6, AAV7, AAV8, AAV9, or AAV10, or derivatives/modifications/pseudotypes thereof). Such AAV serotypes and derivatives/modifications/pseudotypes, and methods of producing such serotypes/derivatives/modifications/pseudotypes are known in the art (see, e.g., Asokan et al., Mol. Ther. 20 (4): 699-708 (2012).

The rAAV particles of the disclosure may be of any serotype, or any combination of serotypes, (e.g., a population of rAAV particles that comprises two or more serotypes, e.g., comprising two or more of rAAV2, rAAV8, and rAAV9 particles). In some embodiments, the rAAV particles are rAAV1, rAAV2, rAAV3, rAAV4, rAAV5, rAAV6, rAAV7, rAAV8, rAAV9, rAAV10, or other rAAV particles, or combinations of two or more thereof). In some embodiments, the rAAV particles are rAAV8 or rAAV9 particles.

In some embodiments, the rAAV particles have an AAV capsid protein of a serotype selected from the group consisting of AAV1, AAV2, AAV3, AAV4, AAV5, AAV6, AAV7, AAV8, AAV9, AAV10, AAV11, AAV12, AAV13, AAV14, AAV15 and AAV16 or a derivative, modification, or pseudotype thereof. In some embodiments, the rAAV particles have an AAV capsid protein of a serotype of AAV8, AAV9, or a derivative, modification, or pseudotype thereof.

The term “cell culture,” refers to cells grown adherent or in suspension, bioreactors, roller bottles, hyperstacks, microspheres, macrospheres, flasks and the like, as well as the components of the supernatant or suspension itself, including but not limited to rAAV particles, cells, cell debris, cellular contaminants, colloidal particles, biomolecules, host cell proteins, nucleic acids, and lipids, and flocculants. Large scale approaches, such as bioreactors, including suspension cultures and adherent cells growing attached to microcarriers or macrocarriers in stirred bioreactors, are also encompassed by the term “cell culture.” Cell culture procedures for both large and small-scale production of proteins are encompassed by the present disclosure. In some embodiments, the term “cell culture” refers to cells grown in suspension. In some embodiments, the term “cell culture” refers to adherent cells grown attached to microcarriers or macrocarriers in stirred bioreactors. In some embodiments, the term “cell culture” refers to cells grown in a perfusion culture. In some embodiments, the term “cell culture” refers to cells grown in an alternating tangential flow (ATF) supported high-density perfusion culture.

The terms “purifying”, “purification”, “separate”, “separating”, “separation”, “isolate”, “isolating”, or “isolation”, as used herein, refer to increasing the degree of purity of a target product, e.g., rAAV particles and rAAV genome from a sample comprising the target product and one or more impurities. Typically, the degree of purity of the target product is increased by removing (completely or partially) at least one impurity from the sample. In some embodiments, the degree of purity of the rAAV in a sample is increased by removing (completely or partially) one or more impurities from the sample by using a method described herein.

“About” modifying, for example, the quantity of an ingredient in the compositions, concentration of an ingredient in the compositions, flow rate, rAAV particle yield, feed volume, salt concentration, and like values, and ranges thereof, employed in the methods provided herein, refers to variation in the numerical quantity that can occur, for example, through typical measuring and handling procedures used for making concentrates or use solutions; through inadvertent error in these procedures; through differences in the manufacture, source, or purity of the ingredients employed to make the compositions or carry out the methods; and like considerations. The term “about” also encompasses amounts that differ due to aging of a composition with a particular initial concentration or mixture. The term “about” also encompasses amounts that differ due to mixing or processing a composition with a particular initial concentration or mixture. Whether or not modified by the term “about” the claims include equivalents to the quantities. In some embodiments, the term “about” refers to ranges of approximately 10-20% greater than or less than the indicated number or range. In further embodiments, “about” refers to plus or minus 10% of the indicated number or range. For example, “about 10%” indicates a range of 9% to 11%.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Where embodiments of the disclosure are described in terms of a Markush group or other grouping of alternatives, the disclosed method encompasses not only the entire group listed as a whole, but also each member of the group individually and all possible subgroups of the main group, and also the main group absent one or more of the group members. The disclosed methods also envisage the explicit exclusion of one or more of any of the group members in the disclosed methods.

### Recombinant Polynucleotides

In some embodiments, the disclosure provides an isolated recombinant polynucleotide encoding one or more helper functions, and an AAV rep gene and an AAV cap gene, wherein the polynucleotide is capable of promoting production of recombinant AAV particles in a host cell, e.g., an HEK cell. In some embodiments, an isolated recombinant polynucleotide described herein comprises (a) a nucleotide sequence encoding an adenovirus E2A DNA binding protein (DBP); (b) a nucleotide sequence encoding an adenovirus E4 polypeptide; (c) a nucleotide sequence encoding an adenovirus VA RNA I, and (d) a nucleotide sequence encoding an AAV rep gene and an AAV cap gene. In some embodiments, the adenovirus E4 polypeptide comprises the E4 ORF6 and ORF7 or the E4 ORF6. In some embodiments, the nucleotide sequence encoding an adenovirus VA RNA I encodes an adenovirus VA RNA I and VA RNA II. In some embodiments, the nucleotide sequence encoding an AAV rep gene and an AAV cap gene comprises a nucleotide sequence encoding a parvovirus p5 promoter and a nucleotide sequence encoding an AAV rep gene and an AAV cap gene, wherein the parvovirus p5 promoter is operably linked to the AAV rep gene and controls the expression of the rep78 and rep68 gene products. In some embodiments, the nucleotide sequence encoding a parvovirus p5 promoter is immediately upstream of the nucleotide sequence encoding an AAV rep gene. In some embodiments, the nucleotide sequence encoding a parvovirus p5 promoter is separated from the nucleotide sequence encoding an AAV rep gene by between about 1 and about 10,000 nucleotides. In some embodiments, the isolated recombinant polynucleotide does not comprise a nucleotide sequence encoding an adenovirus ITR sequence, L3 23K endoprotease, L5 pVI/fibre, and/or L4 pVIII/hexon-associated precursor. In some embodiments, the nucleotide sequence encoding the adenovirus ITR sequence, L3 23K endoprotease, L5 pVI/fibre, and/or L4 pVIII/hexon-associated precursor is the corresponding nucleotide sequence of pAdDeltaF6. In some embodiments, a nucleotide sequence encoding a protein or polypeptide (e.g., E2A DBP, E4 polypeptide, AAV rep and AAV cap), or RNA (e.g., VA RNA I) comprises a promoter operably linked to a nucleotide sequence comprising the coding region for the protein or polypeptide, or RNA. In some embodiments, a nucleotide sequence encoding a protein or polypeptide comprises a promoter and a polyA signal operably linked to a nucleotide sequence comprising the coding region.

In some embodiments, an isolated recombinant polynucleotide described herein comprises a nucleotide sequence encoding an adenovirus E2A DBP, a nucleotide sequence encoding an adenovirus E4 polypeptide and a nucleotide sequence encoding an adenovirus VA RNA I. In some embodiments, an isolated recombinant polynucleotide described herein comprises a nucleotide sequence encoding an adenovirus E2A DBP, and a nucleotide sequence encoding an adenovirus E4 polypeptide. In some embodiments, an isolated recombinant polynucleotide described herein comprises a nucleotide sequence encoding an adenovirus E2A DBP, and a nucleotide sequence encoding an adenovirus VA RNA I. In some embodiments, an isolated recombinant polynucleotide described herein comprises a nucleotide sequence encoding an adenovirus E4 polypeptide and a nucleotide sequence encoding an adenovirus VA RNA I. In some embodiments, an isolated recombinant polynucleotide described herein comprises a nucleotide sequence encoding an adenovirus E2A DBP. In some embodiments, an isolated recombinant polynucleotide described herein comprises a nucleotide sequence encoding an adenovirus E4 polypeptide. In some embodiments, the adenovirus E4 polypeptide comprises the E4 ORF6 and ORF7 or the E4 ORF6. In some embodiments, an isolated recombinant polynucleotide described herein comprises a nucleotide sequence encoding an adenovirus VA RNA I. In some embodiments, the nucleotide sequence encoding an adenovirus VA RNA I encodes an adenovirus VA RNA I and VA RNA II. In some embodiments, the isolated recombinant polynucleotide does not comprise a nucleotide sequence encoding an adenovirus ITR sequence, L3 23K endoprotease, L5 pVI/fibre, and/or L4 pVIII/hexon-associated precursor. In some embodiments, the nucleotide sequence encoding the adenovirus ITR sequence, L3 23K endoprotease, L5 pVI/fibre, and/or L4 pVIII/hexon-associated precursor is the corresponding nucleotide sequence of pAdDeltaF6. In some embodiments of the isolated recombinant polynucleotide, the nucleotide sequence encoding the adenovirus E2A DBP and the nucleotide sequence encoding the adenovirus E4 polypeptide are in opposite 5′ to 3′ orientation. In some embodiments of the isolated recombinant polynucleotide, the nucleotide sequence encoding the adenovirus E2A DBP and the nucleotide sequence encoding the adenovirus E4 polypeptide are in the same 5′ to 3′ orientation. In some embodiments, the nucleotide sequence encoding the adenovirus E2A DBP and the nucleotide sequence encoding the AAV rep gene and AAV cap gene are in opposite 5′ to 3′ orientation.

In some embodiments, an isolated recombinant polynucleotide described herein comprises (a) a nucleotide sequence encoding an adenovirus E2A DBP, (b) a nucleotide sequence encoding an adenovirus E4 polypeptide, (c) a nucleotide sequence encoding an adenovirus VA RNA I and (d) a nucleotide sequence encoding an AAV rep gene and an AAV cap gene, wherein the 5′ to 3′ order of the nucleotide sequences is (d)-(a)-(b)-(c). In some embodiments, the nucleotide sequence encoding the adenovirus E2A DBP and the nucleotide sequence encoding the adenovirus E4 polypeptide are in opposite 5′ to 3′ orientation. In some embodiments, the nucleotide sequence encoding the adenovirus E2A DBP and the nucleotide sequence encoding the AAV rep gene and AAV cap gene are in opposite 5′ to 3′ orientation. In some embodiments, the nucleotide sequence encoding the adenovirus E2A DBP and the nucleotide sequence encoding the adenovirus E4 polypeptide are in opposite 5′ to 3′ orientation and the nucleotide sequence encoding the adenovirus E2A DBP and the nucleotide sequence encoding the AAV rep gene and AAV cap gene are in opposite 5′ to 3′ orientation. In some embodiments, the isolated recombinant polynucleotide does not comprise a nucleotide sequence encoding an adenovirus ITR sequence, L3 23K endoprotease, L5 pVI/fibre, and/or L4 pVIII/hexon-associated precursor. In some embodiments, the adenovirus E4 polypeptide comprises the E4 ORF6 and ORF7 or the E4 ORF6. In some embodiments, the nucleotide sequence encoding an adenovirus VA RNA I encodes an adenovirus VA RNA I and VA RNA II.

In some embodiments, the disclosure provides an isolated recombinant polynucleotide encoding one or more helper functions, an AAV rep gene, an AAV cap gene and a recombinant AAV viral genome, wherein the polynucleotide is capable of promoting production of recombinant AAV particles in a host cell, e.g., an HEK cell. In some embodiments, an isolated recombinant polynucleotide described herein comprises (a) a nucleotide sequence encoding an adenovirus E2A DNA binding protein (DBP); (b) a nucleotide sequence encoding an adenovirus E4 polypeptide; (c) a nucleotide sequence encoding an adenovirus VA RNA I, (d) a nucleotide sequence encoding an AAV rep gene and an AAV cap gene, and (c) a nucleotide sequence encoding a recombinant viral genome comprising at least one AAV inverted terminal repeat (ITR) and a non-AAV nucleic acid sequence encoding a gene product operably linked to sequences which direct expression of the gene product in a target cell. In some embodiments, the adenovirus E4 polypeptide comprises the E4 ORF6 and ORF7 or the E4 ORF6. In some embodiments, the nucleotide sequence encoding an adenovirus VA RNA I encodes an adenovirus VA RNA I and VA RNA II. In some embodiments, the nucleotide sequence encoding an AAV rep gene and an AAV cap gene comprises a nucleotide sequence encoding a parvovirus p5 promoter and a nucleotide sequence encoding an AAV rep gene and an AAV cap gene, wherein the parvovirus p5 promoter is operably linked to the AAV rep gene and controls the expression of the rep78 and rep68 gene products. In some embodiments, the nucleotide sequence encoding a parvovirus p5 promoter is immediately upstream of the nucleotide sequence encoding an AAV rep gene. In some embodiments, the nucleotide sequence encoding a parvovirus p5 promoter is separated from the nucleotide sequence encoding an AAV rep gene by between about 1 and about 10,000 nucleotides. In some embodiments, the isolated recombinant polynucleotide does not comprise a nucleotide sequence encoding an adenovirus ITR sequence, L3 23K endoprotease, L5 pVI/fibre, and/or L4 pVIII/hexon-associated precursor. In some embodiments, the nucleotide sequence encoding the adenovirus ITR sequence, L3 23K endoprotease, L5 pVI/fibre, and/or L4 pVIII/hexon-associated precursor is the corresponding nucleotide sequence of pAdDeltaF6. In some embodiments, a nucleotide sequence encoding a protein or polypeptide (e.g., E2A DBP, E4 polypeptide, AAV rep and AAV cap), or RNA (e.g., VA RNA I) comprises a promoter operably linked to a nucleotide sequence comprising the coding region for the protein or polypeptide, or RNA. In some embodiments, a nucleotide sequence encoding a protein or polypeptide comprises a promoter and a polyA signal operably linked to a nucleotide sequence comprising the coding region. In some embodiments, a recombinant polynucleotide described herein is sufficient by itself to promote production of recombinant AAV particles in a host cell, e.g., an HEK cell.

In some embodiments, an isolated recombinant polynucleotide described herein comprises (a) a nucleotide sequence encoding an adenovirus E2A DBP, (b) a nucleotide sequence encoding an adenovirus E4 polypeptide, (c) a nucleotide sequence encoding an adenovirus VA RNA I, (d) a nucleotide sequence encoding an AAV rep gene and an AAV cap gene and (e) a nucleotide sequence encoding a recombinant AAV viral genome comprising a nucleic acid sequence encoding a gene product, wherein the 5′ to 3′ order of the nucleotide sequences is (d)-(a)-(b)-(e)-(c). In some embodiments, the adenovirus E4 polypeptide comprises the E4 ORF6 and ORF7 or the E4 ORF6. In some embodiments, the nucleotide sequence encoding the adenovirus E2A DBP and the nucleotide sequence encoding the adenovirus E4 polypeptide are in opposite 5′ to 3′ orientation. In some embodiments, the nucleotide sequence encoding the adenovirus E2A DBP and the nucleotide sequence encoding the AAV rep gene and AAV cap gene are in opposite 5′ to 3′ orientation. In some embodiments, the nucleotide sequence encoding the AAV rep gene and AAV cap gene and the nucleic acid sequence encoding a gene product are in opposite 5′ to 3′ orientation. In some embodiments, (i) the nucleotide sequence encoding the adenovirus E2A DBP and the nucleotide sequence encoding the adenovirus E4 polypeptide are in opposite 5′ to 3′ orientation; (ii) the nucleotide sequence encoding the adenovirus E2A DBP and the nucleotide sequence encoding the AAV rep gene and AAV cap gene are in opposite 5′ to 3′ orientation; and (iii) the nucleotide sequence encoding the AAV rep gene and AAV cap gene and the nucleic acid sequence encoding a gene product are in opposite 5′ to 3′ orientation. In some embodiments, the isolated recombinant polynucleotide does not comprise a nucleotide sequence encoding an adenovirus ITR sequence, L3 23K endoprotease, L5 pVI/fibre, and/or L4 pVIII/hexon-associated precursor. In some embodiments, the nucleotide sequence encoding an adenovirus VA RNA I encodes an adenovirus VA RNA I and VA RNA II.

### Adenovirus E2A DNA Binding Protein

In some embodiments, the nucleotide sequence encoding an adenovirus E2A DBP comprises a nucleotide sequence having at least 80%, at least 85%, at least 90%, at least 95%, at least 97%, at least 98%, at least 99% or 100% identity to SEQ ID NO: 1. In some embodiments, the nucleotide sequence encoding an adenovirus E2A DBP comprises a nucleotide sequence having at least 90% identity to SEQ ID NO: 1. In some embodiments, the nucleotide sequence encoding an adenovirus E2A DBP comprises a nucleotide sequence having at least 95% identity to SEQ ID NO: 1. In some embodiments, the nucleotide sequence encoding an adenovirus E2A DBP comprises a nucleotide sequence having at least 98% identity to SEQ ID NO: 1. In some embodiments, the nucleotide sequence encoding an adenovirus E2A DBP comprises SEQ ID NO: 1. In some embodiments, the nucleotide sequence encoding an adenovirus E2A DBP comprises a codon optimized nucleotide sequence. In some embodiments, the nucleotide sequence encoding an adenovirus E2A DBP comprises a nucleotide sequence having at least 80%, at least 85%, at least 90%, at least 95%, at least 97%, at least 98%, at least 99% or 100% identity to SEQ ID NO: 60. In some embodiments, the nucleotide sequence encoding an adenovirus E2A DBP comprises a nucleotide sequence having at least 95% identity to SEQ ID NO: 60. In some embodiments, the nucleotide sequence encoding an adenovirus E2A DBP comprises SEQ ID NO: 60. In some embodiments, the adenovirus E2A DBP polypeptide comprises an amino acid sequence having at least 80%, at least 85%, at least 90%, at least 95%, at least 97%, at least 98%, at least 99% or 100% identity to SEQ ID NO: 45. In some embodiments, the adenovirus E2A DBP polypeptide comprises an amino acid sequence having at least 90% identity to SEQ ID NO: 45. In some embodiments, the adenovirus E2A DBP polypeptide comprises an amino acid sequence having at least 95% identity to SEQ ID NO: 45. In some embodiments, the adenovirus E2A DBP polypeptide comprises an amino acid sequence having at least 98% identity to SEQ ID NO: 45. In some embodiments, the adenovirus E2A DBP polypeptide comprises the amino acid sequence of SEQ ID NO: 45. In some embodiments, the nucleotide sequence encoding an adenovirus E2A DBP is operably linked to a promoter and to a polyA signal.

In some embodiments, the nucleotide sequence encoding an adenovirus E2A DBP comprises a nucleotide sequence encoding a polypeptide comprising an amino acid sequence having at least 80%, at least 85%, at least 90%, at least 95%, at least 97%, at least 98%, at least 99% or 100% identity to SEQ ID NO: 45. In some embodiments, the adenovirus E2A DBP polypeptide comprises an amino acid sequence having at least 90% identity to SEQ ID NO: 45. In some embodiments, the adenovirus E2A DBP polypeptide comprises an amino acid sequence having at least 95% identity to SEQ ID NO: 45. In some embodiments, the adenovirus E2A DBP polypeptide comprises an amino acid sequence having at least 98% identity to SEQ ID NO: 45. In some embodiments, the adenovirus E2A DBP polypeptide comprises the amino acid sequence of SEQ ID NO: 45. In some embodiments, the nucleotide sequence encoding an adenovirus E2A DBP is operably linked to a promoter and to a polyA signal.

In some embodiments, the nucleotide sequence encoding an adenovirus E2A DBP is operably linked to an adenovirus E2A promoter. In some embodiments, the adenovirus E2A promoter comprises a nucleotide sequence comprising at least 80%, at least 85%, at least 90%, at least 95%, at least 97%, at least 98%, at least 99% or 100% identity to SEQ ID NO: 2. In some embodiments, the adenovirus E2A promoter comprises a nucleotide sequence comprising at least 95% identity to SEQ ID NO: 2. In some embodiments, the adenovirus E2A promoter comprises the nucleotide sequence of SEQ ID NO: 2. In some embodiments, the nucleotide sequence encoding an adenovirus E2A DBP is operably linked to a promoter that is not an adenovirus E2A promoter.

In some embodiments, the nucleotide sequence encoding an adenovirus E2A DBP operably linked to an adenovirus E2A promoter, and optionally a polyA signal, encompasses, from 3′ to 5′, the adenovirus E2A promoter, an adenovirus L4 22K/33K gene, an adenovirus L4 100 k/hexon assembly gene, the nucleotide sequence encoding an adenovirus E2A DBP and optionally the polyA signal. In some embodiments, the relative orientation of the adenovirus E2A promoter, adenovirus L4 22K/33K gene, adenovirus L4 100 k/hexon assembly gene, nucleotide sequence encoding an adenovirus E2A DBP and optional polyA signal is the same as in pAdDeltaF6. In some embodiments, the nucleotide sequence encoding an adenovirus E2A DBP operably linked to an adenovirus E2A promoter comprises a nucleotide sequence having at least 80%, at least 85%, at least 90%, at least 95%, at least 97%, at least 98%, at least 99% or 100% identity to SEQ ID NO: 3. In some embodiments, the nucleotide sequence encoding an adenovirus E2A DBP operably linked to an adenovirus E2A promoter comprises a nucleotide sequence having at least 90% identity to SEQ ID NO: 3. In some embodiments, the nucleotide sequence encoding an adenovirus E2A DBP operably linked to an adenovirus E2A promoter comprises a nucleotide sequence having at least 95% identity to SEQ ID NO: 3. In some embodiments, the nucleotide sequence encoding an adenovirus E2A DBP operably linked to an adenovirus E2A promoter comprises a nucleotide sequence having at least 98% identity to SEQ ID NO: 3. In some embodiments, the nucleotide sequence encoding an adenovirus E2A DBP operably linked to an adenovirus E2A promoter comprises the nucleotide sequence of SEQ ID NO: 3. In some embodiments, the nucleotide sequence encoding an adenovirus E2A DBP operably linked to an adenovirus E2A promoter and polyA signal comprises a nucleotide sequence having at least 80%, at least 85%, at least 90%, at least 95%, at least 97%, at least 98%, at least 99% or 100% identity to SEQ ID NO: 4. In some embodiments, the nucleotide sequence encoding an adenovirus E2A DBP operably linked to an adenovirus E2A promoter and polyA signal comprises a nucleotide sequence having at least 90% identity to SEQ ID NO: 4. In some embodiments, the nucleotide sequence encoding an adenovirus E2A DBP operably linked to an adenovirus E2A promoter and polyA signal comprises a nucleotide sequence having at least 95% identity to SEQ ID NO: 4. In some embodiments, the nucleotide sequence encoding an adenovirus E2A DBP operably linked to an adenovirus E2A promoter and polyA signal comprises a nucleotide sequence having at least 98% identity to SEQ ID NO: 4. In some embodiments, the nucleotide sequence encoding an adenovirus E2A DBP operably linked to an adenovirus E2A promoter and polyA signal comprises the nucleotide sequence of SEQ ID NO: 4.

In some embodiments, the nucleotide sequence encoding an adenovirus E2A DBP operably linked to an adenovirus E2A promoter encompasses, from 3′ to 5′, the adenovirus E2A promoter, an adenovirus L4 22K/33K gene, an adenovirus L4 100 k/hexon assembly gene, the nucleotide sequence encoding an adenovirus E2A DBP, wherein the adenovirus L4 100 k/hexon assembly gene comprises an N terminal deletion of the L4 100 k/hexon assembly polypeptide. In some embodiments, the N terminal deletion does not affect the L4 100 k/hexon assembly promoter. In some embodiments, the N terminal deletion corresponds to the sequence of SEQ ID NO: 21. In some embodiments, the relative orientation of the adenovirus E2A promoter, adenovirus L4 22K/33K gene, adenovirus L4 100 k/hexon assembly gene and nucleotide sequence encoding an adenovirus E2A DBP is the same as in pAdDeltaF6. In some embodiments, the nucleotide sequence encoding an adenovirus E2A DBP operably linked to an adenovirus E2A promoter comprises a nucleotide sequence having at least 80%, at least 85%, at least 90%, at least 95%, at least 97%, at least 98%, at least 99% or 100% identity to SEQ ID NO: 22. In some embodiments, the nucleotide sequence encoding an adenovirus E2A DBP operably linked to an adenovirus E2A promoter comprises a nucleotide sequence having at least 90% identity to SEQ ID NO: 22. In some embodiments, the nucleotide sequence encoding an adenovirus E2A DBP operably linked to an adenovirus E2A promoter comprises a nucleotide sequence having at least 95% identity to SEQ ID NO: 22. In some embodiments, the nucleotide sequence encoding an adenovirus E2A DBP operably linked to an adenovirus E2A promoter comprises a nucleotide sequence having at least 98% identity to SEQ ID NO: 22. In some embodiments, the nucleotide sequence encoding an adenovirus E2A DBP operably linked to an adenovirus E2A promoter comprises the nucleotide sequence of SEQ ID NO: 22. In some embodiments, the nucleotide sequence encoding an adenovirus E2A DBP and adenovirus E2A promoter further comprises an operably linked polyA signal.

In some embodiments, the nucleotide sequence encoding an adenovirus E2A DBP operably linked to an adenovirus E2A promoter encompasses, from 3′ to 5′, the adenovirus E2A promoter, an adenovirus L4 22K/33K gene, an adenovirus L4 100 k/hexon assembly gene, the nucleotide sequence encoding an adenovirus E2A DBP, wherein the adenovirus L4 100 k/hexon assembly gene comprises a mutation in the start codon of the L4 100 k/hexon assembly polypeptide. In some embodiments, the relative orientation of the adenovirus E2A promoter, adenovirus L4 22K/33K gene, adenovirus L4 100 k/hexon assembly gene and nucleotide sequence encoding an adenovirus E2A DBP is the same as in pAdDeltaF6. In some embodiments, the nucleotide sequence encoding an adenovirus E2A DBP operably linked to an adenovirus E2A promoter comprises a nucleotide sequence having at least 80%, at least 85%, at least 90%, at least 95%, at least 97%, at least 98%, at least 99% or 100% identity to SEQ ID NO: 23. In some embodiments, the nucleotide sequence encoding an adenovirus E2A DBP operably linked to an adenovirus E2A promoter comprises a nucleotide sequence having at least 90% identity to SEQ ID NO: 23. In some embodiments, the nucleotide sequence encoding an adenovirus E2A DBP operably linked to an adenovirus E2A promoter comprises a nucleotide sequence having at least 95% identity to SEQ ID NO: 23. In some embodiments, the nucleotide sequence encoding an adenovirus E2A DBP operably linked to an adenovirus E2A promoter comprises a nucleotide sequence having at least 98% identity to SEQ ID NO: 23. In some embodiments, the nucleotide sequence encoding an adenovirus E2A DBP operably linked to an adenovirus E2A promoter comprises the nucleotide sequence of SEQ ID NO: 23. In some embodiments, the nucleotide sequence encoding an adenovirus E2A DBP and adenovirus E2A promoter further comprises an operably linked polyA signal.

In some embodiments, the nucleotide sequence encoding an adenovirus E2A DBP operably linked to an adenovirus E2A promoter encompasses, from 3′ to 5′, the adenovirus E2A promoter, an adenovirus L4 22K/33K gene, an adenovirus L4 100 k/hexon assembly gene, the nucleotide sequence encoding an adenovirus E2A DBP, wherein the adenovirus L4 22K/33K gene comprises a mutation in the start codon of the L4 22K/33K polypeptide. In some embodiments, the relative orientation of the adenovirus E2A promoter, adenovirus L4 22K/33K gene, adenovirus L4 100 k/hexon assembly gene and nucleotide sequence encoding an adenovirus E2A DBP is the same as in pAdDeltaF6. In some embodiments, the nucleotide sequence encoding an adenovirus E2A DBP operably linked to an adenovirus E2A promoter comprises a nucleotide sequence having at least 80%, at least 85%, at least 90%, at least 95%, at least 97%, at least 98%, at least 99% or 100% identity to SEQ ID NO: 24. In some embodiments, the nucleotide sequence encoding an adenovirus E2A DBP operably linked to an adenovirus E2A promoter comprises a nucleotide sequence having at least 90% identity to SEQ ID NO: 24. In some embodiments, the nucleotide sequence encoding an adenovirus E2A DBP operably linked to an adenovirus E2A promoter comprises a nucleotide sequence having at least 95% identity to SEQ ID NO: 24. In some embodiments, the nucleotide sequence encoding an adenovirus E2A DBP operably linked to an adenovirus E2A promoter comprises a nucleotide sequence having at least 98% identity to SEQ ID NO: 24. In some embodiments, the nucleotide sequence encoding an adenovirus E2A DBP operably linked to an adenovirus E2A promoter comprises the nucleotide sequence of SEQ ID NO: 24. In some embodiments, the nucleotide sequence encoding an adenovirus E2A DBP and adenovirus E2A promoter further comprises an operably linked polyA signal.

In some embodiments, the nucleotide sequence encoding an adenovirus E2A DBP operably linked to an adenovirus E2A promoter encompasses, from 3′ to 5′, the adenovirus E2A promoter, an adenovirus L4 22K/33K gene, an adenovirus L4 100 k/hexon assembly gene, the nucleotide sequence encoding an adenovirus E2A DBP, wherein the L4 100 k/hexon assembly gene comprises an N terminal deletion of the L4 100 k/hexon assembly polypeptide that encompasses the start codon of L4 100 k/hexon assembly polypeptide but does not encompass the start codon of the L4 22K/33K polypeptide. In some embodiments, the nucleotide sequence encoding an adenovirus E2A DBP operably linked to an adenovirus E2A promoter encompasses, from 3′ to 5′, the adenovirus E2A promoter, an adenovirus L4 22K/33K gene, an adenovirus L4 100 k/hexon assembly gene, the nucleotide sequence encoding an adenovirus E2A DBP, wherein the L4 100 k/hexon assembly gene comprises an N terminal deletion of the L4 100 k/hexon assembly polypeptide, wherein all or part of the L4 100 k/hexon assembly polypeptide is deleted without disrupting the L4 22K/33K start codon. In some embodiments, the nucleotide sequence encoding an adenovirus E2A DBP operably linked to an adenovirus E2A promoter encompasses, from 3′ to 5′, the adenovirus E2A promoter, an adenovirus L4 22K/33K gene, an adenovirus L4 100 k/hexon assembly gene, the nucleotide sequence encoding an adenovirus E2A DBP, wherein the L4 100 k/hexon assembly gene comprises an N terminal deletion of the L4 100 k/hexon assembly polypeptide that encompasses the start codon of L4 100 k/hexon assembly polypeptide but does not encompass the L4 22K/33K promoter. In some embodiments, the nucleotide sequence encoding an adenovirus E2A DBP operably linked to an adenovirus E2A promoter encompasses, from 3′ to 5′, the adenovirus E2A promoter, an adenovirus L4 22K/33K gene, an adenovirus L4 100 k/hexon assembly gene, the nucleotide sequence encoding an adenovirus E2A DBP, wherein the L4 100 k/hexon assembly gene comprises an N terminal deletion of the L4 100 k/hexon assembly polypeptide that starts at the start codon of L4 100 k/hexon assembly polypeptide and ends immediately adjacent to the L4 22K/33K promoter. In some embodiments, the relative orientation of the adenovirus E2A promoter, adenovirus L4 22K/33K gene, adenovirus L4 100 k/hexon assembly gene and nucleotide sequence encoding an adenovirus E2A DBP is the same as in pAdDeltaF6. In some embodiments, the nucleotide sequence encoding an adenovirus E2A DBP and adenovirus E2A promoter further comprises an operably linked polyA signal.

In some embodiments, the nucleotide sequence encoding an adenovirus E2A DBP is operably linked to a CMV immediate early promoter. In some embodiments, the nucleotide sequence encoding an adenovirus E2A DBP is operably linked to an engineered CMV immediate early promoter, or a transcriptionally active fragment or portion thereof. In some embodiments, the CMV immediate early promoter comprises a nucleotide sequence of having at least 80%, at least 85%, at least 90%, at least 95%, at least 97%, at least 98%, at least 99% or 100% identity to SEQ ID NO: 121. In some embodiments, the CMV immediate early promoter comprises the nucleotide sequence of SEQ ID NO: 121. Engineered CMV immediate early promoters or transcriptionally active fragments or portions thereof are known to one of skill, for example, as disclosed in International Application No. PCT/US2023/061014, filed Jan. 20, 2023, which is incorporated herein by reference in its entirety.

In some embodiments, the nucleotide sequence encoding an adenovirus E2A DBP is operably linked to an inducible promoter.

### Adenovirus E4 Polypeptide

In some embodiments, the nucleotide sequence encoding an adenovirus E4 polypeptide encodes an E4 polypeptide comprising E4 ORF6. In some embodiments, the nucleotide sequence encoding an adenovirus E4 ORF6 polypeptide comprises a nucleotide sequence having at least 80%, at least 85%, at least 90%, at least 95%, at least 97%, at least 98%, at least 99% or 100% identity to SEQ ID NO: 6. In some embodiments, the nucleotide sequence encoding an adenovirus E4 ORF6 polypeptide comprises a nucleotide sequence having at least 90% identity to SEQ ID NO: 6. In some embodiments, the nucleotide sequence encoding an adenovirus E4 ORF6 polypeptide comprises a nucleotide sequence having at least 95% identity to SEQ ID NO: 6. In some embodiments, the nucleotide sequence encoding an adenovirus E4 ORF6 polypeptide comprises a nucleotide sequence having at least 98% identity to SEQ ID NO: 6. In some embodiments, the nucleotide sequence encoding an adenovirus E4 ORF6 polypeptide comprises SEQ ID NO: 6. In some embodiments, the adenovirus E4 ORF6 polypeptide comprises an amino acid sequence having at least 80%, at least 85%, at least 90%, at least 95%, at least 97%, at least 98%, at least 99% or 100% identity to SEQ ID NO: 46. In some embodiments, the adenovirus E4 ORF6 polypeptide comprises an amino acid sequence having at least 90% identity to SEQ ID NO: 46. In some embodiments, the adenovirus E4 ORF6 polypeptide comprises an amino acid sequence having at least 95% identity to SEQ ID NO: 46. In some embodiments, the adenovirus E4 ORF6 polypeptide comprises an amino acid sequence having at least 98% identity to SEQ ID NO: 46. In some embodiments, the adenovirus E4 ORF6 polypeptide comprises the amino acid sequence of SEQ ID NO: 46. In some embodiments, the nucleotide sequence encoding an adenovirus E4 ORF6 polypeptide is operably linked to a promoter and to a polyA signal.

In some embodiments, the nucleotide sequence encoding an adenovirus E4 ORF6 polypeptide comprises a nucleotide sequence encoding a polypeptide comprising an amino acid sequence having at least 80%, at least 85%, at least 90%, at least 95%, at least 97%, at least 98%, at least 99% or 100% identity to SEQ ID NO: 46. In some embodiments, the adenovirus E4 ORF6 polypeptide comprises an amino acid sequence having at least 90% identity to SEQ ID NO: 46. In some embodiments, the adenovirus E4 ORF6 polypeptide comprises an amino acid sequence having at least 95% identity to SEQ ID NO: 46. In some embodiments, the adenovirus E4 ORF6 polypeptide comprises an amino acid sequence having at least 98% identity to SEQ ID NO: 46. In some embodiments, the adenovirus E4 ORF6 polypeptide comprises the amino acid sequence of SEQ ID NO: 46. In some embodiments, the nucleotide sequence encoding an adenovirus E4 ORF6 polypeptide is operably linked to a promoter and to a polyA signal.

In some embodiments, the nucleotide sequence encoding an adenovirus E4 ORF6 polypeptide is operably linked to an adenovirus E4 promoter. In some embodiments, the adenovirus E4 promoter comprises a nucleotide sequence comprising at least 80%, at least 85%, at least 90%, at least 95%, at least 97%, at least 98%, at least 99% or 100% identity to SEQ ID NO: 5. In some embodiments, the adenovirus E4 promoter comprises a nucleotide sequence comprising at least 95% identity to SEQ ID NO: 5. In some embodiments, the adenovirus E4 promoter comprises the nucleotide sequence of SEQ ID NO: 5. In some embodiments, the nucleotide sequence encoding an adenovirus E4 ORF6 polypeptide is operably linked to a promoter that is not an adenovirus E4 promoter.

In some embodiments, the nucleotide sequence encoding an adenovirus E4 polypeptide encodes an E4 polypeptide comprising E4 ORF6 and ORF7. E4 ORF7 is also referenced as E4 ORF6/7 in the art. In some embodiments, the nucleotide sequence encoding an adenovirus E4 ORF6 and ORF7 polypeptide comprises a nucleotide sequence having at least 80%, at least 85%, at least 90%, at least 95%, at least 97%, at least 98%, at least 99% or 100% identity to SEQ ID NO: 8. In some embodiments, the nucleotide sequence encoding an adenovirus E4 ORF6 and ORF7 polypeptide comprises a nucleotide sequence having at least 90% identity to SEQ ID NO: 8. In some embodiments, the nucleotide sequence encoding an adenovirus E4 ORF6 and ORF7 polypeptide comprises a nucleotide sequence having at least 95% identity to SEQ ID NO: 8. In some embodiments, the nucleotide sequence encoding an adenovirus E4 ORF6 and ORF7 polypeptide comprises a nucleotide sequence having at least 98% identity to SEQ ID NO: 8. In some embodiments, the nucleotide sequence encoding an adenovirus E4 ORF6 and ORF7 polypeptide comprises SEQ ID NO: 8. In some embodiments, the nucleotide sequence encoding an adenovirus E4 ORF6 and ORF7 polypeptide comprises a codon optimized nucleotide sequence. In some embodiments, the nucleotide sequence encoding an adenovirus E4 ORF6 and ORF7 polypeptide comprises a nucleotide sequence having at least 80%, at least 85%, at least 90%, at least 95%, at least 97%, at least 98%, at least 99% or 100% identity to SEQ ID NO: 61. In some embodiments, the nucleotide sequence encoding an adenovirus E4 ORF6 and ORF7 polypeptide comprises a nucleotide sequence having at least 95% identity to SEQ ID NO: 61. In some embodiments, the nucleotide sequence encoding an adenovirus E4 ORF6 and ORF7 polypeptide comprises SEQ ID NO: 61. A skilled artisan understands that the polynucleotide of SEQ ID NO: 8 encodes two alternatively spliced mRNAs, one each for ORF6 and ORF7 (i.e., ORF6/7). The ORF6 mRNA comprises the nucleotide sequence corresponding to residues 1-885 of SEQ ID NO: 8. The ORF7 (i.e., ORF6/7) mRNA comprises the nucleotide sequences corresponding to residues 1-174 (exon 1) and 886-1164 (exon 2) of SEQ ID NO:8. In some embodiments, the adenovirus E4 ORF6 and ORF7 polypeptide comprises an amino acid sequence having at least 80%, at least 85%, at least 90%, at least 95%, at least 97%, at least 98%, at least 99% or 100% identity to SEQ ID NO: 46 and 120, respectively. In some embodiments, the adenovirus E4 ORF6 polypeptide comprises an amino acid sequence having at least 90% identity to SEQ ID NO: 46. In some embodiments, the adenovirus E4 ORF6 polypeptide comprises an amino acid sequence having at least 95% identity to SEQ ID NO: 46. In some embodiments, the adenovirus E4 ORF6 polypeptide comprises an amino acid sequence having at least 98% identity to SEQ ID NO: 46. In some embodiments, the adenovirus E4 ORF6 polypeptide comprises the amino acid sequence of SEQ ID NO: 46. In some embodiments, the adenovirus E4 ORF7 polypeptide comprises an amino acid sequence having at least 90% identity to SEQ ID NO: 120. In some embodiments, the adenovirus E4 ORF7 polypeptide comprises an amino acid sequence having at least 95% identity to SEQ ID NO: 120. In some embodiments, the adenovirus E4 ORF7 polypeptide comprises an amino acid sequence having at least 98% identity to SEQ ID NO: 120. In some embodiments, the adenovirus E4 ORF7 polypeptide comprises the amino acid sequence of SEQ ID NO: 120. In some embodiments, the nucleotide sequence encoding an adenovirus E4 ORF6 and ORF7 polypeptide is operably linked to a promoter and to a polyA signal.

In some embodiments, the nucleotide sequence encoding an adenovirus E4 ORF6 and ORF7 polypeptide comprises a nucleotide sequence encoding a ORF6 polypeptide comprising an amino acid sequence having at least 80%, at least 85%, at least 90%, at least 95%, at least 97%, at least 98%, at least 99% or 100% identity to SEQ ID NO: 46 and an ORF7 polypeptide comprising an amino acid sequence having at least 80%, at least 85%, at least 90%, at least 95%, at least 97%, at least 98%, at least 99% or 100% identity to SEQ ID NO: 120. In some embodiments, the adenovirus E4 ORF6 polypeptide comprises an amino acid sequence having at least 90% identity to SEQ ID NO: 46. In some embodiments, the adenovirus E4 ORF6 polypeptide comprises an amino acid sequence having at least 95% identity to SEQ ID NO: 46. In some embodiments, the adenovirus E4 ORF6 polypeptide comprises an amino acid sequence having at least 98% identity to SEQ ID NO: 46. In some embodiments, the adenovirus E4 ORF6 polypeptide comprises the amino acid sequence of SEQ ID NO: 46. In some embodiments, the adenovirus E4 ORF7 polypeptide comprises an amino acid sequence having at least 90% identity to SEQ ID NO: 120. In some embodiments, the adenovirus E4 ORF7 polypeptide comprises an amino acid sequence having at least 95% identity to SEQ ID NO: 120. In some embodiments, the adenovirus E4 ORF7 polypeptide comprises an amino acid sequence having at least 98% identity to SEQ ID NO: 120. In some embodiments, the adenovirus E4 ORF7 polypeptide comprises the amino acid sequence of SEQ ID NO: 120. In some embodiments, the nucleotide sequence encoding an adenovirus E4 ORF6 and ORF7 polypeptide is operably linked to a promoter and to a polyA signal.

In some embodiments, the nucleotide sequence encoding an adenovirus E4 ORF6 and ORF7 polypeptide is operably linked to an adenovirus E4 promoter. In some embodiments, the adenovirus E4 promoter comprises a nucleotide sequence comprising at least 80%, at least 85%, at least 90%, at least 95%, at least 97%, at least 98%, at least 99% or 100% identity to SEQ ID NO: 5. In some embodiments, the adenovirus E4 promoter comprises a nucleotide sequence comprising at least 95% identity to SEQ ID NO: 5. In some embodiments, the adenovirus E4 promoter comprises the nucleotide sequence of SEQ ID NO: 5. In some embodiments, the nucleotide sequence encoding an adenovirus E4 ORF6 and ORF7 polypeptide is operably linked to a promoter that is not an adenovirus E4 promoter.

In some embodiments, the nucleotide sequence encoding an adenovirus polypeptide is operably linked to a CMV immediate early promoter. In some embodiments, the nucleotide sequence encoding an adenovirus E4 polypeptide is operably linked to an engineered CMV immediate early promoter, or a transcriptionally active fragment or portion thereof. In some embodiments, the CMV immediate early promoter comprises a nucleotide sequence of having at least 80%, at least 85%, at least 90%, at least 95%, at least 97%, at least 98%, at least 99% or 100% identity to SEQ ID NO: 121. In some embodiments, the CMV immediate early promoter comprises the nucleotide sequence of SEQ ID NO: 121. Engineered CMV immediate early promoters or transcriptionally active fragments or portions thereof are known to one of skill, for example, as disclosed in International Application No. PCT/US2023/061014, filed Jan. 20, 2023, which is incorporated herein by reference in its entirety. In some embodiments, the adenovirus E4 polypeptide comprises the E4 ORF6 and ORF7 or the E4 ORF6.

In some embodiments, the nucleotide sequence encoding an adenovirus E4 polypeptide is operably linked to an inducible promoter. In some embodiments, the adenovirus E4 polypeptide comprises the E4 ORF6 and ORF7 or the E4 ORF6.

### Adenovirus VA RNA

In some embodiments, the nucleotide sequence encoding an adenovirus VA RNA I comprises a nucleotide sequence having at least 80%, at least 85%, at least 90%, at least 95%, at least 97%, at least 98%, at least 99% or 100% identity to SEQ ID NO: 54. In some embodiments, the nucleotide sequence encoding an adenovirus VA RNA I comprises a nucleotide sequence having at least 90% identity to SEQ ID NO: 54. In some embodiments, the nucleotide sequence encoding an adenovirus VA RNA I comprises a nucleotide sequence having at least 95% identity to SEQ ID NO: 54. In some embodiments, the nucleotide sequence encoding an adenovirus VA RNA I comprises a nucleotide sequence having at least 98% identity to SEQ ID NO: 54. In some embodiments, the nucleotide sequence encoding an adenovirus VA RNA I comprises SEQ ID NO: 54.

In some embodiments, the nucleotide sequence encoding an adenovirus VA RNA II comprises a nucleotide sequence having at least 80%, at least 85%, at least 90%, at least 95%, at least 97%, at least 98%, at least 99% or 100% identity to SEQ ID NO: 55. In some embodiments, the nucleotide sequence encoding an adenovirus VA RNA II comprises a nucleotide sequence having at least 90% identity to SEQ ID NO: 55. In some embodiments, the nucleotide sequence encoding an adenovirus VA RNA II comprises a nucleotide sequence having at least 95% identity to SEQ ID NO: 55. In some embodiments, the nucleotide sequence encoding an adenovirus VA RNA II comprises a nucleotide sequence having at least 98% identity to SEQ ID NO: 55. In some embodiments, the nucleotide sequence encoding an adenovirus VA RNA II comprises SEQ ID NO: 55.

In some embodiments, the nucleotide sequence encoding an adenovirus VA RNA I and VA RNA II comprises a nucleotide sequence having at least 80%, at least 85%, at least 90%, at least 95%, at least 97%, at least 98%, at least 99% or 100% identity to SEQ ID NO: 9. In some embodiments, the nucleotide sequence encoding an adenovirus VA RNA I and VA RNA II comprises a nucleotide sequence having at least 90% identity to SEQ ID NO: 9. In some embodiments, the nucleotide sequence encoding an adenovirus VA RNA I and VA RNA II comprises a nucleotide sequence having at least 95% identity to SEQ ID NO: 9. In some embodiments, the nucleotide sequence encoding an adenovirus VA RNA I and VA RNA II comprises a nucleotide sequence having at least 98% identity to SEQ ID NO: 9. In some embodiments, the nucleotide sequence encoding an adenovirus VA RNA I and VA RNA II comprises SEQ ID NO: 9.

### Polynucleotides Encoding E2A DBP, E4 Polypeptide and VA RNA

In some embodiments, an isolated recombinant polynucleotide described herein comprises a fragment comprising a nucleotide sequence encoding an adenovirus E2A DBP, a nucleotide sequence encoding an adenovirus E4 polypeptide and a nucleotide sequence encoding an adenovirus VA RNA I. In some embodiments, the adenovirus E4 polypeptide comprises E4 ORF6 and ORF7. In some embodiments, the adenovirus E4 polypeptide comprises the E4 ORF6. In some embodiments, the nucleotide sequence encoding an adenovirus VA RNA I encodes an adenovirus VA RNA I and VA RNA II. In some embodiments, the promoter expressing the adenovirus E2A DBP and the promoter expressing the adenovirus E4 polypeptide are the same. In some embodiments, the promoter expressing the adenovirus E2A DBP and the promoter expressing the adenovirus E4 polypeptide are different.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 10. In some embodiments, the fragment comprises a nucleotide sequence having at least 90% identity to SEQ ID NO: 10. In some embodiments, the fragment comprises a nucleotide sequence having at least 95% identity to SEQ ID NO: 10. In some embodiments, the fragment comprises a nucleotide sequence having at least 98% identity to SEQ ID NO: 10. In some embodiments, the fragment comprises the nucleotide sequence of SEQ ID NO: 10. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 11. In some embodiments, the fragment comprises a nucleotide sequence having at least 90% identity to SEQ ID NO: 11. In some embodiments, the fragment comprises a nucleotide sequence having at least 95% identity to SEQ ID NO: 11. In some embodiments, the fragment comprises a nucleotide sequence having at least 98% identity to SEQ ID NO: 11. In some embodiments, the fragment comprises the nucleotide sequence of SEQ ID NO: 11. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 56. In some embodiments, the fragment comprises a nucleotide sequence having at least 90% identity to SEQ ID NO: 56. In some embodiments, the fragment comprises a nucleotide sequence having at least 95% identity to SEQ ID NO: 56. In some embodiments, the fragment comprises a nucleotide sequence having at least 98% identity to SEQ ID NO: 56. In some embodiments, the fragment comprises the nucleotide sequence of SEQ ID NO: 56. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 57. In some embodiments, the fragment comprises a nucleotide sequence having at least 90% identity to SEQ ID NO: 57. In some embodiments, the fragment comprises a nucleotide sequence having at least 95% identity to SEQ ID NO: 57. In some embodiments, the fragment comprises a nucleotide sequence having at least 98% identity to SEQ ID NO: 57. In some embodiments, the fragment comprises the nucleotide sequence of SEQ ID NO: 57. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 25. In some embodiments, the fragment comprises a nucleotide sequence having at least 90% identity to SEQ ID NO: 25. In some embodiments, the fragment comprises a nucleotide sequence having at least 95% identity to SEQ ID NO: 25. In some embodiments, the fragment comprises a nucleotide sequence having at least 98% identity to SEQ ID NO: 25. In some embodiments, the fragment comprises the nucleotide sequence of SEQ ID NO: 25. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 26. In some embodiments, the fragment comprises a nucleotide sequence having at least 90% identity to SEQ ID NO: 26. In some embodiments, the fragment comprises a nucleotide sequence having at least 95% identity to SEQ ID NO: 26. In some embodiments, the fragment comprises a nucleotide sequence having at least 98% identity to SEQ ID NO: 26. In some embodiments, the fragment comprises the nucleotide sequence of SEQ ID NO: 26. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 27. In some embodiments, the fragment comprises a nucleotide sequence having at least 90% identity to SEQ ID NO: 27. In some embodiments, the fragment comprises a nucleotide sequence having at least 95% identity to SEQ ID NO: 27. In some embodiments, the fragment comprises a nucleotide sequence having at least 98% identity to SEQ ID NO: 27. In some embodiments, the fragment comprises the nucleotide sequence of SEQ ID NO: 27. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 28. In some embodiments, the fragment comprises a nucleotide sequence having at least 90% identity to SEQ ID NO: 28. In some embodiments, the fragment comprises a nucleotide sequence having at least 95% identity to SEQ ID NO: 28. In some embodiments, the fragment comprises a nucleotide sequence having at least 98% identity to SEQ ID NO: 28. In some embodiments, the fragment comprises the nucleotide sequence of SEQ ID NO: 28. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 29. In some embodiments, the fragment comprises a nucleotide sequence having at least 90% identity to SEQ ID NO: 29. In some embodiments, the fragment comprises a nucleotide sequence having at least 95% identity to SEQ ID NO: 29. In some embodiments, the fragment comprises a nucleotide sequence having at least 98% identity to SEQ ID NO: 29. In some embodiments, the fragment comprises the nucleotide sequence of SEQ ID NO: 29. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 30. In some embodiments, the fragment comprises a nucleotide sequence having at least 90% identity to SEQ ID NO: 30. In some embodiments, the fragment comprises a nucleotide sequence having at least 95% identity to SEQ ID NO: 30. In some embodiments, the fragment comprises a nucleotide sequence having at least 98% identity to SEQ ID NO: 30. In some embodiments, the fragment comprises the nucleotide sequence of SEQ ID NO: 30. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 31. In some embodiments, the fragment comprises a nucleotide sequence having at least 90% identity to SEQ ID NO: 31. In some embodiments, the fragment comprises a nucleotide sequence having at least 95% identity to SEQ ID NO: 31. In some embodiments, the fragment comprises a nucleotide sequence having at least 98% identity to SEQ ID NO: 31. In some embodiments, the fragment comprises the nucleotide sequence of SEQ ID NO: 31. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 32. In some embodiments, the fragment comprises a nucleotide sequence having at least 90% identity to SEQ ID NO: 32. In some embodiments, the fragment comprises a nucleotide sequence having at least 95% identity to SEQ ID NO: 32. In some embodiments, the fragment comprises a nucleotide sequence having at least 98% identity to SEQ ID NO: 32. In some embodiments, the fragment comprises the nucleotide sequence of SEQ ID NO: 32. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 33. In some embodiments, the fragment comprises a nucleotide sequence having at least 90% identity to SEQ ID NO: 33. In some embodiments, the fragment comprises a nucleotide sequence having at least 95% identity to SEQ ID NO: 33. In some embodiments, the fragment comprises a nucleotide sequence having at least 98% identity to SEQ ID NO: 33. In some embodiments, the fragment comprises the nucleotide sequence of SEQ ID NO: 33. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 34. In some embodiments, the fragment comprises a nucleotide sequence having at least 90% identity to SEQ ID NO: 34. In some embodiments, the fragment comprises a nucleotide sequence having at least 95% identity to SEQ ID NO: 34. In some embodiments, the fragment comprises a nucleotide sequence having at least 98% identity to SEQ ID NO: 34. In some embodiments, the fragment comprises the nucleotide sequence of SEQ ID NO: 34. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 106. In some embodiments, the fragment comprises a nucleotide sequence having at least 90% identity to SEQ ID NO: 106. In some embodiments, the fragment comprises a nucleotide sequence having at least 95% identity to SEQ ID NO: 106. In some embodiments, the fragment comprises a nucleotide sequence having at least 98% identity to SEQ ID NO: 106. In some embodiments, the fragment comprises the nucleotide sequence of SEQ ID NO: 106. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 107. In some embodiments, the fragment comprises a nucleotide sequence having at least 90% identity to SEQ ID NO: 107. In some embodiments, the fragment comprises a nucleotide sequence having at least 95% identity to SEQ ID NO: 107. In some embodiments, the fragment comprises a nucleotide sequence having at least 98% identity to SEQ ID NO: 107. In some embodiments, the fragment comprises the nucleotide sequence of SEQ ID NO: 107. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 108. In some embodiments, the fragment comprises a nucleotide sequence having at least 90% identity to SEQ ID NO: 108. In some embodiments, the fragment comprises a nucleotide sequence having at least 95% identity to SEQ ID NO: 108. In some embodiments, the fragment comprises a nucleotide sequence having at least 98% identity to SEQ ID NO: 108. In some embodiments, the fragment comprises the nucleotide sequence of SEQ ID NO: 108. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 109. In some embodiments, the fragment comprises a nucleotide sequence having at least 90% identity to SEQ ID NO: 109. In some embodiments, the fragment comprises a nucleotide sequence having at least 95% identity to SEQ ID NO: 109. In some embodiments, the fragment comprises a nucleotide sequence having at least 98% identity to SEQ ID NO: 109. In some embodiments, the fragment comprises the nucleotide sequence of SEQ ID NO: 109. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 56 or 57. In some embodiments, the fragment comprises a nucleotide sequence having at least 90% identity to SEQ ID NO: 56 or 57. In some embodiments, the fragment comprises a nucleotide sequence having at least 95% identity to SEQ ID NO: 56 or 57. In some embodiments, the fragment comprises a nucleotide sequence having at least 98% identity to SEQ ID NO: 56 or 57. In some embodiments, the fragment comprises the nucleotide sequence of SEQ ID NO: 56 or 57. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 106 or 107. In some embodiments, the fragment comprises a nucleotide sequence having at least 90% identity to SEQ ID NO: 106 or 107. In some embodiments, the fragment comprises a nucleotide sequence having at least 95% identity to SEQ ID NO: 106 or 107. In some embodiments, the fragment comprises a nucleotide sequence having at least 98% identity to SEQ ID NO: 106 or 107. In some embodiments, the fragment comprises the nucleotide sequence of SEQ ID NO: 106 or 107. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 108 or 109. In some embodiments, the fragment comprises a nucleotide sequence having at least 90% identity to SEQ ID NO: 108 or 109. In some embodiments, the fragment comprises a nucleotide sequence having at least 95% identity to SEQ ID NO: 108 or 109. In some embodiments, the fragment comprises a nucleotide sequence having at least 98% identity to SEQ ID NO: 108 or 109. In some embodiments, the fragment comprises the nucleotide sequence of SEQ ID NO: 108 or 109. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 122-130 or 131. In some embodiments, the fragment comprises a nucleotide sequence having at least 90% identity to SEQ ID NO: 122-130 or 131. In some embodiments, the fragment comprises a nucleotide sequence having at least 95% identity to SEQ ID NO: 122-130 or 131. In some embodiments, the fragment comprises a nucleotide sequence having at least 98% identity to SEQ ID NO: 122-130 or 131. In some embodiments, the fragment comprises the nucleotide sequence of SEQ ID NO: 122-130 or 131. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 140-158, 165, 170, 175, 180, 185, 190, 195, 200, 205, 210, 215, 220, 225, 230, 235, 240, 245, 250, 255, or 260. In some embodiments, the fragment comprises a nucleotide sequence having at least 90% identity to SEQ ID NO: 140-158, 165, 170, 175, 180, 185, 190, 195, 200, 205, 210, 215, 220, 225, 230, 235, 240, 245, 250, 255, or 260. In some embodiments, the fragment comprises a nucleotide sequence having at least 95% identity to SEQ ID NO: 140-158, 165, 170, 175, 180, 185, 190, 195, 200, 205, 210, 215, 220, 225, 230, 235, 240, 245, 250, 255, or 260. In some embodiments, the fragment comprises a nucleotide sequence having at least 98% identity to SEQ ID NO: 140-158, 165, 170, 175, 180, 185, 190, 195, 200, 205, 210, 215, 220, 225, 230, 235, 240, 245, 250, 255, or 260. In some embodiments, the fragment comprises the nucleotide sequence of SEQ ID NO: 140-158, 165, 170, 175, 180, 185, 190, 195, 200, 205, 210, 215, 220, 225, 230, 235, 240, 245, 250, 255, or 260. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 145 or 146. In some embodiments, the fragment comprises a nucleotide sequence having at least 90% identity to SEQ ID NO: 145 or 146. In some embodiments, the fragment comprises a nucleotide sequence having at least 95% identity to SEQ ID NO: 145 or 146. In some embodiments, the fragment comprises a nucleotide sequence having at least 98% identity to SEQ ID NO: 145 or 146. In some embodiments, the fragment comprises the nucleotide sequence of SEQ ID NO: 145 or 146. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60.

In some embodiments, an isolated recombinant polynucleotide described herein comprises a fragment comprising a nucleotide sequence encoding an adenovirus E2A DBP, a nucleotide sequence encoding an adenovirus E4 ORF6 polypeptide and a nucleotide sequence encoding an adenovirus VA RNA I and VA RNA II. In some embodiments, the fragment comprises a nucleotide sequence having at least 80%, at least 85%, at least 90%, at least 95%, at least 97%, at least 98%, at least 99% or 100% identity to SEQ ID NO: 156, 157 or 158. In some embodiments, the fragment comprises a nucleotide sequence having at least 90% identity to SEQ ID NO: 156, 157 or 158. In some embodiments, the fragment comprises a nucleotide sequence having at least 95% identity to SEQ ID NO: 156, 157 or 158. In some embodiments, the fragment comprises a nucleotide sequence having at least 98% identity to SEQ ID NO: 156, 157 or 158. In some embodiments, the fragment comprises the nucleotide sequence of SEQ ID NO: 156, 157 or 158. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60.

### Polynucleotides Encoding E2A DBP and E4 Polypeptide

In some embodiments, an isolated recombinant polynucleotide described herein comprises a fragment comprising a nucleotide sequence encoding an adenovirus E2A DBP and a nucleotide sequence encoding an adenovirus E4 polypeptide. In some embodiments, the adenovirus E4 polypeptide comprises E4 ORF6 and ORF7. In some embodiments, the adenovirus E4 polypeptide comprises the E4 ORF6. In some embodiments, the promoter expressing the adenovirus E2A DBP and the promoter expressing the adenovirus E4 polypeptide are the same. In some embodiments, the promoter expressing the adenovirus E2A DBP and the promoter expressing the adenovirus E4 polypeptide are different.

In some embodiments, an isolated recombinant polynucleotide described herein comprises a fragment comprising a nucleotide sequence encoding an adenovirus E2A DBP and a nucleotide sequence encoding an adenovirus E4 ORF6 and ORF7 polypeptide. In some embodiments, the fragment comprises a nucleotide sequence having at least 80%, at least 85%, at least 90%, at least 95%, at least 97%, at least 98%, at least 99% or 100% identity to SEQ ID NO: 110. In some embodiments, the fragment comprises a nucleotide sequence having at least 90% identity to SEQ ID NO: 110. In some embodiments, the fragment comprises a nucleotide sequence having at least 95% identity to SEQ ID NO: 110. In some embodiments, the fragment comprises a nucleotide sequence having at least 98% identity to SEQ ID NO: 110. In some embodiments, the fragment comprises the nucleotide sequence of SEQ ID NO: 110. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP and a nucleotide sequence encoding an adenovirus E4 ORF6 and ORF7 polypeptide. In some embodiments, the fragment comprises a nucleotide sequence having at least 80%, at least 85%, at least 90%, at least 95%, at least 97%, at least 98%, at least 99% or 100% identity to SEQ ID NO: 111. In some embodiments, the fragment comprises a nucleotide sequence having at least 90% identity to SEQ ID NO: 111. In some embodiments, the fragment comprises a nucleotide sequence having at least 95% identity to SEQ ID NO: 111. In some embodiments, the fragment comprises a nucleotide sequence having at least 98% identity to SEQ ID NO: 111. In some embodiments, the fragment comprises the nucleotide sequence of SEQ ID NO: 111. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP and a nucleotide sequence encoding an adenovirus E4 ORF6 and ORF7 polypeptide. In some embodiments, the fragment comprises a nucleotide sequence having at least 80%, at least 85%, at least 90%, at least 95%, at least 97%, at least 98%, at least 99% or 100% identity to SEQ ID NO: 112. In some embodiments, the fragment comprises a nucleotide sequence having at least 90% identity to SEQ ID NO: 112. In some embodiments, the fragment comprises a nucleotide sequence having at least 95% identity to SEQ ID NO: 112. In some embodiments, the fragment comprises a nucleotide sequence having at least 98% identity to SEQ ID NO: 112. In some embodiments, the fragment comprises the nucleotide sequence of SEQ ID NO: 112. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP and a nucleotide sequence encoding an adenovirus E4 ORF6 and ORF7 polypeptide. In some embodiments, the fragment comprises a nucleotide sequence having at least 80%, at least 85%, at least 90%, at least 95%, at least 97%, at least 98%, at least 99% or 100% identity to SEQ ID NO: 113. In some embodiments, the fragment comprises a nucleotide sequence having at least 90% identity to SEQ ID NO: 113. In some embodiments, the fragment comprises a nucleotide sequence having at least 95% identity to SEQ ID NO: 113. In some embodiments, the fragment comprises a nucleotide sequence having at least 98% identity to SEQ ID NO: 113. In some embodiments, the fragment comprises the nucleotide sequence of SEQ ID NO: 113. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP and a nucleotide sequence encoding an adenovirus E4 ORF6 and ORF7 polypeptide. In some embodiments, the fragment comprises a nucleotide sequence having at least 80%, at least 85%, at least 90%, at least 95%, at least 97%, at least 98%, at least 99% or 100% identity to SEQ ID NO: 114. In some embodiments, the fragment comprises a nucleotide sequence having at least 90% identity to SEQ ID NO: 114. In some embodiments, the fragment comprises a nucleotide sequence having at least 95% identity to SEQ ID NO: 114. In some embodiments, the fragment comprises a nucleotide sequence having at least 98% identity to SEQ ID NO: 114. In some embodiments, the fragment comprises the nucleotide sequence of SEQ ID NO: 114. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP and a nucleotide sequence encoding an adenovirus E4 ORF6 and ORF7 polypeptide. In some embodiments, the fragment comprises a nucleotide sequence having at least 80%, at least 85%, at least 90%, at least 95%, at least 97%, at least 98%, at least 99% or 100% identity to SEQ ID NO: 115. In some embodiments, the fragment comprises a nucleotide sequence having at least 90% identity to SEQ ID NO: 115. In some embodiments, the fragment comprises a nucleotide sequence having at least 95% identity to SEQ ID NO: 115. In some embodiments, the fragment comprises a nucleotide sequence having at least 98% identity to SEQ ID NO: 115. In some embodiments, the fragment comprises the nucleotide sequence of SEQ ID NO: 115. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP and a nucleotide sequence encoding an adenovirus E4 ORF6 and ORF7 polypeptide. In some embodiments, the fragment comprises a nucleotide sequence having at least 80%, at least 85%, at least 90%, at least 95%, at least 97%, at least 98%, at least 99% or 100% identity to SEQ ID NO: 116. In some embodiments, the fragment comprises a nucleotide sequence having at least 90% identity to SEQ ID NO: 116. In some embodiments, the fragment comprises a nucleotide sequence having at least 95% identity to SEQ ID NO: 116. In some embodiments, the fragment comprises a nucleotide sequence having at least 98% identity to SEQ ID NO: 116. In some embodiments, the fragment comprises the nucleotide sequence of SEQ ID NO: 116. In some embodiments, the nucleotide sequence encoding an adenovirus E4 ORF6 and ORF7 polypeptide comprises a codon optimized coding region. In some embodiments, the codon optimized E4 ORF6 and ORF7 polypeptide coding region comprises the nucleotide sequence of SEQ ID NO: 61.

In some embodiments, an isolated recombinant polynucleotide described herein comprises a fragment comprising a nucleotide sequence encoding an adenovirus E2A DBP and a nucleotide sequence encoding an adenovirus E4 ORF6 and ORF7 polypeptide. In some embodiments, the fragment comprises a nucleotide sequence having at least 80%, at least 85%, at least 90%, at least 95%, at least 97%, at least 98%, at least 99% or 100% identity to SEQ ID NO: 117. In some embodiments, the fragment comprises a nucleotide sequence having at least 90% identity to SEQ ID NO: 117. In some embodiments, the fragment comprises a nucleotide sequence having at least 95% identity to SEQ ID NO: 117. In some embodiments, the fragment comprises a nucleotide sequence having at least 98% identity to SEQ ID NO: 117. In some embodiments, the fragment comprises the nucleotide sequence of SEQ ID NO: 117. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60.

In some embodiments, an isolated recombinant polynucleotide described herein comprises a fragment comprising a nucleotide sequence encoding an adenovirus E2A DBP and a nucleotide sequence encoding an adenovirus E4 ORF6 and ORF7 polypeptide. In some embodiments, the fragment comprises a nucleotide sequence having at least 80%, at least 85%, at least 90%, at least 95%, at least 97%, at least 98%, at least 99% or 100% identity to SEQ ID NO: 277. In some embodiments, the fragment comprises a nucleotide sequence having at least 90% identity to SEQ ID NO: 277. In some embodiments, the fragment comprises a nucleotide sequence having at least 95% identity to SEQ ID NO: 277. In some embodiments, the fragment comprises a nucleotide sequence having at least 98% identity to SEQ ID NO: 277. In some embodiments, the fragment comprises the nucleotide sequence of SEQ ID NO: 277. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60.

In some embodiments, an isolated recombinant polynucleotide described herein comprises a fragment comprising a nucleotide sequence encoding an adenovirus E2A DBP and a nucleotide sequence encoding an adenovirus E4 ORF6 and ORF7 polypeptide. In some embodiments, the fragment comprises a nucleotide sequence having at least 80%, at least 85%, at least 90%, at least 95%, at least 97%, at least 98%, at least 99% or 100% identity to SEQ ID NO: 278. In some embodiments, the fragment comprises a nucleotide sequence having at least 90% identity to SEQ ID NO: 278. In some embodiments, the fragment comprises a nucleotide sequence having at least 95% identity to SEQ ID NO: 278. In some embodiments, the fragment comprises a nucleotide sequence having at least 98% identity to SEQ ID NO: 278. In some embodiments, the fragment comprises the nucleotide sequence of SEQ ID NO: 278. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60.

In some embodiments, an isolated recombinant polynucleotide described herein comprises a fragment comprising a nucleotide sequence encoding an adenovirus E2A DBP and a nucleotide sequence encoding an adenovirus E4 ORF6 and ORF7 polypeptide. In some embodiments, the fragment comprises a nucleotide sequence having at least 80%, at least 85%, at least 90%, at least 95%, at least 97%, at least 98%, at least 99% or 100% identity to SEQ ID NO: 279. In some embodiments, the fragment comprises a nucleotide sequence having at least 90% identity to SEQ ID NO: 279. In some embodiments, the fragment comprises a nucleotide sequence having at least 95% identity to SEQ ID NO: 279. In some embodiments, the fragment comprises a nucleotide sequence having at least 98% identity to SEQ ID NO: 279. In some embodiments, the fragment comprises the nucleotide sequence of SEQ ID NO: 279. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60.

In some embodiments, an isolated recombinant polynucleotide described herein comprises a fragment comprising a nucleotide sequence encoding an adenovirus E2A DBP and a nucleotide sequence encoding an adenovirus E4 ORF6 polypeptide. In some embodiments, the fragment comprises a nucleotide sequence having at least 80%, at least 85%, at least 90%, at least 95%, at least 97%, at least 98%, at least 99% or 100% identity to SEQ ID NO: 280, 281 or 282. In some embodiments, the fragment comprises a nucleotide sequence having at least 90% identity to SEQ ID NO: 280, 281 or 282. In some embodiments, the fragment comprises a nucleotide sequence having at least 95% identity to SEQ ID NO: 280, 281 or 282. In some embodiments, the fragment comprises a nucleotide sequence having at least 98% identity to SEQ ID NO: 280, 281 or 282. In some embodiments, the fragment comprises the nucleotide sequence of SEQ ID NO: 280, 281 or 282. In some embodiments, the nucleotide sequence encoding an adenovirus E2A DBP comprises a codon optimized coding region. In some embodiments, the codon optimized E2A DBP coding region comprises the nucleotide sequence of SEQ ID NO: 60.

### AAV Rep Gene and an AAV Cap Gene

In some embodiments, the nucleotide sequence encoding an AAV rep gene and an AAV cap gene comprises a nucleotide sequence encoding a parvovirus p5 promoter and a nucleotide sequence encoding an AAV rep gene and an AAV cap gene, wherein the parvovirus p5 promoter is operably linked to the AAV rep gene and controls the expression of the rep78 and rep68 gene products. In some embodiments, the p5 promoter is positioned between about 10 and about 10,000 nucleotides upstream from the AAV rep start codon.

In some embodiments, the p5 promoter is positioned between about 5,000 and about 10,000 nucleotides upstream from the AAV rep start codon. In some embodiments, the p5 promoter is positioned between about 1,000 and about 5,000 nucleotides upstream from the AAV rep start codon. In some embodiments, the p5 promoter is positioned between about 1,000 and about 4,000 nucleotides upstream from the AAV rep start codon. In some embodiments, the p5 promoter is positioned between about 1,000 and about 3,000 nucleotides upstream from the AAV rep start codon. In some embodiments, the p5 promoter is positioned between about 2,000 and about 5,000 nucleotides upstream from the AAV rep start codon. In some embodiments, the p5 promoter is positioned between about 2,000 and about 4,000 nucleotides upstream from the AAV rep start codon. In some embodiments, the p5 promoter is positioned between about 2,000 and about 3,000 nucleotides upstream from the AAV rep start codon.

In some embodiments, the nucleotide sequences encoding E2A DBP, E4 polypeptide and/or VA RNA are positioned between the p5 promoter and the AAV rep start codon upstream from the AAV rep start codon. In some embodiments, the nucleotide sequences encoding E2A DBP, E4 polypeptide and VA RNA are positioned between the p5 promoter and the AAV rep start codon upstream from the AAV rep start codon. In some embodiments, the nucleotide sequences encoding E2A DBP, E4 polypeptide or VA RNA are not positioned between the p5 promoter and the AAV rep start codon upstream from the AAV rep start codon.

In some embodiments, the parvovirus p5 promoter is an AAV p5 promoter. In some embodiments, the AAV p5 promoter comprises a nucleotide sequence having at least 80%, at least 85%, at least 90%, at least 95%, at least 97%, at least 98%, at least 99% or 100% identity to SEQ ID NO: 62. In some embodiments, the AAV p5 promoter comprises a nucleotide sequence having at least 90% identity to SEQ ID NO: 62. In some embodiments, the AAV p5 promoter comprises a nucleotide sequence having at least 95% identity to SEQ ID NO: 62. In some embodiments, the AAV p5 promoter comprises a nucleotide sequence having at least 98% identity to SEQ ID NO: 62. In some embodiments, the AAV p5 promoter comprises the nucleotide sequence of SEQ ID NO: 62.

In some embodiments, the nucleotide sequence encodes an AAV rep gene and an AAV cap gene wherein the AAV rep gene and the AAV cap gene have the same serotype. In some embodiments, the AAV rep gene and the AAV cap gene have different serotypes. In some embodiments, the nucleotide sequence encodes an AAV2 rep gene and an AAV2 cap gene. In some embodiments, the nucleotide sequence encodes an AAV2 rep gene and an AAV6 cap gene. In some embodiments, the nucleotide sequence encodes an AAV2 rep gene and an AAV8 cap gene. In some embodiments, the nucleotide sequence encodes an AAV2 rep gene and an AAV9 cap gene.

In some embodiments, the nucleotide sequence encodes an AAV2 rep gene. In some embodiments, the AAV2 rep gene comprises a nucleotide sequence having at least 80%, at least 85%, at least 90%, at least 95%, at least 97%, at least 98%, at least 99% or 100% identity to SEQ ID NO: 63.

In some embodiments, the AAV2 rep gene comprises a nucleotide sequence having at least 90% identity to SEQ ID NO: 63. In some embodiments, the AAV2 rep gene comprises a nucleotide sequence having at least 95% identity to SEQ ID NO: 63. In some embodiments, AAV2 rep gene comprises a nucleotide sequence having at least 98% identity to SEQ ID NO: 63. In some embodiments, AAV2 rep gene comprises the nucleotide sequence of SEQ ID NO: 63.

In some embodiments, the nucleotide sequence encodes an AAV cap gene comprising a serotype selected from the group consisting of AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the AAV cap gene comprises a serotype selected from the group consisting of AAV8, AAV9, AAV.rh10, AAV.rh20, AAV.rh39, AAV.Rh74, AAV.RHM4-1, AAV.hu32, and AAV.hu37. In some embodiments, the AAV cap gene comprises a serotype selected from the group consisting of AAV8 or AAV9 serotype.

In some embodiments, the AAV cap gene comprises the AAV2 serotype. In some embodiments, the AAV2 cap gene comprises a nucleotide sequence having at least 80%, at least 85%, at least 90%, at least 95%, at least 97%, at least 98%, at least 99% or 100% identity to SEQ ID NO: 64. In some embodiments, the AAV2 cap gene comprises a nucleotide sequence having at least 90% identity to SEQ ID NO: 64. In some embodiments, the AAV2 cap gene comprises a nucleotide sequence having at least 95% identity to SEQ ID NO: 64. In some embodiments, AAV2 cap gene comprises a nucleotide sequence having at least 98% identity to SEQ ID NO: 64. In some embodiments, AAV2 cap gene comprises the nucleotide sequence of SEQ ID NO: 64.

In some embodiments, the AAV cap gene comprises the AAV6 serotype. In some embodiments, the AAV6 cap gene comprises a nucleotide sequence having at least 80%, at least 85%, at least 90%, at least 95%, at least 97%, at least 98%, at least 99% or 100% identity to SEQ ID NO: 65. In some embodiments, the AAV6 cap gene comprises a nucleotide sequence having at least 90% identity to SEQ ID NO: 65. In some embodiments, the AAV6 cap gene comprises a nucleotide sequence having at least 95% identity to SEQ ID NO: 65. In some embodiments, AAV6 cap gene comprises a nucleotide sequence having at least 98% identity to SEQ ID NO: 65. In some embodiments, AAV6 cap gene comprises the nucleotide sequence of SEQ ID NO: 65.

In some embodiments, the AAV cap gene comprises the AAV8 serotype. In some embodiments, the AAV8 cap gene comprises a nucleotide sequence having at least 80%, at least 85%, at least 90%, at least 95%, at least 97%, at least 98%, at least 99% or 100% identity to SEQ ID NO: 66. In some embodiments, the AAV8 cap gene comprises a nucleotide sequence having at least 90% identity to SEQ ID NO: 66. In some embodiments, the AAV8 cap gene comprises a nucleotide sequence having at least 95% identity to SEQ ID NO: 66. In some embodiments, AAV8 cap gene comprises a nucleotide sequence having at least 98% identity to SEQ ID NO: 66. In some embodiments, AAV8 cap gene comprises the nucleotide sequence of SEQ ID NO: 66.

In some embodiments, the AAV cap gene comprises the AAV9 serotype. In some embodiments, the AAV9 cap gene comprises a nucleotide sequence having at least 80%, at least 85%, at least 90%, at least 95%, at least 97%, at least 98%, at least 99% or 100% identity to SEQ ID NO: 67. In some embodiments, the AAV9 cap gene comprises a nucleotide sequence having at least 90% identity to SEQ ID NO: 67. In some embodiments, the AAV9 cap gene comprises a nucleotide sequence having at least 95% identity to SEQ ID NO: 67. In some embodiments, AAV9 cap gene comprises a nucleotide sequence having at least 98% identity to SEQ ID NO: 67. In some embodiments, AAV9 cap gene comprises the nucleotide sequence of SEQ ID NO: 67.

In some embodiments, the nucleotide sequence encoding an AAV rep gene and an AAV cap gene comprises a nucleotide sequence having at least 80%, at least 85%, at least 90%, at least 95%, at least 97%, at least 98%, at least 99% or 100% identity to SEQ ID NO: 68-70 or 71. In some embodiments, the nucleotide sequence encoding an AAV rep gene and an AAV cap gene comprises a nucleotide sequence having at least 90% identity to SEQ ID NO: 68-70 or 71. In some embodiments, the nucleotide sequence encoding an AAV rep gene and an AAV cap gene comprises a nucleotide sequence having at least 95% identity to SEQ ID NO: 68-70 or 71. In some embodiments, the nucleotide sequence encoding an AAV rep gene and an AAV cap gene comprises a nucleotide sequence having at least 98% identity to SEQ ID NO: 68-70 or 71. In some embodiments, the nucleotide sequence encoding an AAV rep gene and an AAV cap gene comprises the nucleotide sequence of SEQ ID NO: 68-70 or 71.

A skilled artisan understands that AAV nucleotide sequences an AAV cap gene also encode the assembly-activating protein (AAP) and membrane-associated assembly protein (mAAP). Because the cap polypeptide is encoded in a different reading frame than the AAP and mAAP polypeptides, a skilled artisan understands that the nucleotide sequences encoding a cap gene described herein can comprise a non-sense mutation introducing a stop codon into the reading frame encoding the AAP and mAAP polypeptide without also disrupting the reading frame encoding the cap polypeptide. In some embodiment, the nucleotide sequence encoding an AAV cap gene comprises a mutation that affects the expression of the mAAP polypeptide. In some embodiments, the nucleotide sequence encoding an AAV cap gene comprises a mutation that introduces a stop codon into the reading frame of the mAAP polypeptide resulting in the production of a truncated mAAP polypeptide. In some embodiments, the nucleotide sequence encoding an AAV cap gene comprises one or more mutations that introduce one or more stop codons into the reading frame of the mAAP polypeptide. In some embodiments, the nucleotide sequence encoding an AAV cap gene comprises two, three, four, five or six mutations that introduce two, three, four, five or six, respectively, stop codons into the reading frame of the mAAP polypeptide. In some embodiments, the nucleotide sequence encoding an AAV cap gene comprises six mutations that introduce six stop codons into the reading frame of the mAAP polypeptide. In some embodiments, the one or more mutations affecting the mAAP reading frame comprise a non-sense mutation for one or more of amino acid residues S39, L78, E90, L100, L106 and L110 of mAAP. In some embodiments, the one or more mutations affecting the mAAP reading frame comprise six non-sense mutations for amino acid residues S39, L78, E90, L100, L106 and L110 of mAAP. In some embodiments, the one or more mutations affecting the mAAP reading frame correspond to the mutation present in any one of the pHRC #17 through #34 packaging vectors. In some embodiments, the mutations affecting the mAAP reading frame correspond to the mutations present in the pHRC #38 or #39 packaging vector. In some embodiments, the nucleotide sequence encoding an AAV cap gene comprises a mutation that disrupts the first initiation codon of the mAAP polypeptide. In some embodiments, the nucleotide sequence encoding an AAV cap gene comprises a mutation that disrupts translation initiation for the mAAP polypeptide. In some embodiments, the nucleotide sequence encoding the cap gene comprises the nucleotide sequence encoding the cap gene of any one of the pHRC #17 through #34 packaging vectors. In some embodiments, the nucleotide sequence encoding the cap gene comprises the nucleotide sequence encoding the cap gene of the pHRC #38 or #39 packaging vector.

In some embodiment, the nucleotide sequence encoding an AAV cap gene comprises one or mutations that affect the expression of the mAAP polypeptide. In some embodiments, the AAV cap gene comprises a nucleotide sequence having at least 80%, at least 85%, at least 90%, at least 95%, at least 97%, at least 98%, at least 99% or 100% identity to SEQ ID NO: 166, 171, 176, 181, 186, 191, 196, 201, 206, 211, 216, 221, 226, 231, 236, 241, 246, 251, 256, or 261. In some embodiments, the AAV cap gene comprises a nucleotide sequence having at least 90% identity to SEQ ID NO: 166, 171, 176, 181, 186, 191, 196, 201, 206, 211, 216, 221, 226, 231, 236, 241, 246, 251, 256, or 261. In some embodiments, the AAV cap gene comprises a nucleotide sequence having at least 95% identity to SEQ ID NO: 166, 171, 176, 181, 186, 191, 196, 201, 206, 211, 216, 221, 226, 231, 236, 241, 246, 251, 256, or 261. In some embodiments, AAV cap gene comprises a nucleotide sequence having at least 98% identity to SEQ ID NO: 166, 171, 176, 181, 186, 191, 196, 201, 206, 211, 216, 221, 226, 231, 236, 241, 246, 251, 256, or 261. In some embodiments, AAV cap gene comprises the nucleotide sequence of SEQ ID NO: 166, 171, 176, 181, 186, 191, 196, 201, 206, 211, 216, 221, 226, 231, 236, 241, 246, 251, 256, or 261.

In some embodiment, the nucleotide sequence encoding an AAV rep gene and an AAV cap gene comprises one or mutations that affect the expression of the mAAP polypeptide. In some embodiments, the nucleotide sequence encoding an AAV rep gene and an AAV cap gene comprises a nucleotide sequence having at least 80%, at least 85%, at least 90%, at least 95%, at least 97%, at least 98%, at least 99% or 100% identity to SEQ ID NO: 167, 172, 187, 192, 197, 202, 207, 212, 217, 222, 227, 232, 237, 242, 247, 252, 257, or 262. In some embodiments, the nucleotide sequence encoding an AAV rep gene and an AAV cap gene comprises a nucleotide sequence having at least 90% identity to SEQ ID NO: 167, 172, 187, 192, 197, 202, 207, 212, 217, 222, 227, 232, 237, 242, 247, 252, 257, or 262. In some embodiments, the nucleotide sequence encoding an AAV rep gene and an AAV cap gene comprises a nucleotide sequence having at least 95% identity to SEQ ID NO: 167, 172, 187, 192, 197, 202, 207, 212, 217, 222, 227, 232, 237, 242, 247, 252, 257, or 262. In some embodiments, the nucleotide sequence encoding an AAV rep gene and an AAV cap gene comprises a nucleotide sequence having at least 98% identity to SEQ ID NO: 167, 172, 187, 192, 197, 202, 207, 212, 217, 222, 227, 232, 237, 242, 247, 252, 257, or 262. In some embodiments, the nucleotide sequence encoding an AAV rep gene and an AAV cap gene comprises the nucleotide sequence of SEQ ID NO: 167, 172, 187, 192, 197, 202, 207, 212, 217, 222, 227, 232, 237, 242, 247, 252, 257, or 262.

### Recombinant Viral Genome

In some embodiments, the nucleotide sequence encoding a recombinant viral genome comprises at least one AAV inverted terminal repeat (ITR) and a non-AAV nucleic acid sequence encoding a gene product operably linked to sequences which direct expression of the gene product in a target cell. In certain embodiments, the gene product is from Tables 2A-2C. In some embodiments, the rAAV genome comprises the following components: (1) AAV inverted terminal repeats that flank an expression cassette; (2) regulatory control elements, such as a) promoter/enhancers, b) a polyA signal, and c) optionally an intron; and (3) nucleic acid sequences coding for a transgene. In other embodiments for expressing an intact or substantially intact monoclonal antibody (mAb), the rAAV genome comprises the following components: (1) AAV inverted terminal repeats that flank an expression cassette; (2) regulatory control elements, such as a) promoter/enhancers, b) a polyA signal, and c) optionally an intron; and (3) nucleic acid sequences coding for the light chain Fab and heavy chain Fab of the antibody, or at least the heavy chain or light chain Fab, and optionally a heavy chain Fc region. In still other embodiments for expressing an intact or substantially intact mAb, the rAAV genome comprises the following components: (1) AAV inverted terminal repeats that flank an expression cassette; (2) regulatory control elements, such as a) promoter/enhancers, b) a polyA signal, and c) optionally an intron; and (3) nucleic acid sequences coding for the heavy chain Fab of an anti-VEGF (e.g., sevacizumab, ranibizumab, bevacizumab, and brolucizumab), anti-EpoR (e.g., LKA-651), anti-ALK1 (e.g., ascrinvacumab), anti-C5 (e.g., tesidolumab and eculizumab), anti-CD105 (e.g., carotuximab), anti-CC1Q (e.g., ANX-007), anti-TNFα (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab and sarilumab), anti-IL4R (e.g., dupilumab), anti-IL17A (e.g., ixekizumab and secukinumab), anti-IL-5 (e.g., mepolizumab), anti-IL12/IL23 (e.g., ustekinumab), anti-CD19 (e.g., inebilizumab), anti-ITGF7 mAb (e.g., etrolizumab), anti-SOST mAb (e.g., romosozumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., densomab), anti-PCSK9 (e.g., alirocumab and evolocumab), anti-ANGPTL3 (e.g., evinacumab*), anti-OxPL (e.g., E06), anti-fD (e.g., lampalizumab), or anti-MMP9 (e.g., andecaliximab); optionally an Fc polypeptide of the same isotype as the native form of the therapeutic antibody, such as an IgG isotype amino acid sequence IgG1, IgG2 or IgG4 or modified Fc thereof; and the light chain of an anti-VEGF (e.g., sevacizumab, ranibizumab, bevacizumab, and brolucizumab), anti-EpoR (e.g., LKA-651), anti-ALK1 (e.g., ascrinvacumab), anti-C5 (e.g., tesidolumab and eculizumab), anti-CD105 or anti-ENG (e.g., carotuximab), anti-CC1Q (e.g., ANX-007), anti-TNFα (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab and sarilumab), anti-IL4R (e.g., dupilumab), anti-IL17A (e.g., ixekizumab and secukinumab), anti-IL-5 (e.g., mepolizumab), anti-IL12/IL23 (e.g., ustekinumab), anti-CD19 (e.g., inebilizumab), anti-ITGF7 mAb (e.g., etrolizumab), anti-SOST mAb (e.g., romosozumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., densomab), anti-PCSK9 (e.g., alirocumab and evolocumab), anti-ANGPTL3 (e.g., evinacumab), anti-OxPL (e.g., E06), anti-fD (e.g., lampalizumab), or anti-MMP9 (e.g., andecaliximab); wherein the heavy chain (Fab and optionally Fc region) and the light chain are separated by a self-cleaving furin (F)/F2A or flexible linker, ensuring expression of equal amounts of the heavy and the light chain polypeptides.

In some embodiments, an isolated polynucleotide disclosed herein comprises a nucleotide sequence encoding a recombinant viral genome that encodes an anti-VEGF Fab, optionally wherein the isolated polynucleotide further comprises a nucleotide sequence encoding an AAV8 cap gene. In more specific embodiments, the anti-VEGF Fab is ranibizumab. In some embodiments, an isolated polynucleotide disclosed herein comprises a nucleotide sequence encoding a recombinant viral genome that encodes iduronidase (IDUA), optionally wherein the isolated polynucleotide further comprises a nucleotide sequence encoding an AAV9 cap gene. In some embodiments, an isolated polynucleotide disclosed herein comprises a nucleotide sequence encoding a recombinant viral genome that encodes iduronate 2-sulfatase (IDS), optionally wherein the isolated polynucleotide further comprises a nucleotide sequence encoding an AAV9 cap gene. In some embodiments, an isolated polynucleotide disclosed herein comprises a nucleotide sequence encoding a recombinant viral genome that encodes a low-density lipoprotein receptor (LDLR), optionally wherein the isolated polynucleotide further comprises a nucleotide sequence encoding an AAV8 cap gene. In some embodiments, an isolated polynucleotide disclosed herein comprises a nucleotide sequence encoding a recombinant viral genome that encodes a tripeptidyl peptidase 1 (TPP1) protein, optionally wherein the isolated polynucleotide further comprises a nucleotide sequence encoding an AAV9 cap gene. In some embodiments, an isolated polynucleotide disclosed herein comprises a nucleotide sequence encoding a recombinant viral genome that encodes a non-membrane associated splice variant of VEGF receptor 1 (sFlt-1). In some embodiments, an isolated polynucleotide disclosed herein comprises a nucleotide sequence encoding a recombinant viral genome that encodes a gamma-sarcoglycan, Rab Escort Protein 1 (REP1/CHM), retinoid isomerohydrolase (RPE65), cyclic nucleotide gated channel alpha 3 (CNGA3), cyclic nucleotide gated channel beta 3 (CNGB3), aromatic L-amino acid decarboxylase (AADC), lysosome-associated membrane protein 2 isoform B (LAMP2B), Factor VIII, Factor IX, retinitis pigmentosa GTPase regulator (RPGR), retinoschisin (RS1), sarcoplasmic reticulum calcium ATPase (SERCA2a), aflibercept, battenin (CLN3), transmembrane ER protein (CLN6), glutamic acid decarboxylase (GAD), Glial cell line-derived neurotrophic factor (GDNF), aquaporin 1 (AQP1), dystrophin, microdystrophin, myotubularin 1 (MTM1), follistatin (FST), glucose-6-phosphatase (G6Pasc), apolipoprotein A2 (APOA2), uridine diphosphate glucuronosyl transferase 1A1 (UGT1A1), arylsulfatase B (ARSB), N-acetyl-alpha-glucosaminidase (NAGLU), alpha-glucosidase (GAA), alpha-galactosidase (GLA), beta-galactosidase (GLB1), lipoprotein lipase (LPL), alpha 1-antitrypsin (AAT), phosphodiesterase 6B (PDE6B), ornithine carbamoyltransferase 90TC), survival motor neuron (SMN1), survival motor neuron (SMN2), neurturin (NRTN), Neurotrophin-3 (NT-3/NTF3), porphobilinogen deaminase (PBGD), nerve growth factor (NGF), mitochondrially encoded NADH: ubiquinone oxidoreductase core subunit 4 (MT-ND4), protective protein cathepsin A (PPCA), dysferlin, MER proto-oncogene, tyrosine kinase (MERTK), cystic fibrosis transmembrane conductance regulator (CFTR), or tumor necrosis factor receptor (TNFR)-immunoglobulin (IgG1) Fc fusion.

### Boca Virus NP1 and NS2 Polypeptides

In some embodiments, an isolated recombinant polynucleotide described herein further comprises a nucleotide sequence encoding a Boca virus NP1 and NS2 polypeptides. In some embodiments, the nucleotide sequence encoding the Boca virus NP1 and NS2 polypeptides has at least 80%, at least 85%, at least 90%, at least 95%, at least 97%, at least 98%, at least 99% or 100% identity to SEQ ID NO: 12. In some embodiments, the nucleotide sequence encoding the Boca virus NP1 and NS2 polypeptides has at least 90% identity to SEQ ID NO: 12. In some embodiments, the nucleotide sequence encoding the Boca virus NP1 and NS2 polypeptides has at least 95% identity to SEQ ID NO: 12. In some embodiments, the nucleotide sequence encoding the Boca virus NP1 and NS2 polypeptides has at least 98% identity to SEQ ID NO: 12. In some embodiments, the nucleotide sequence encoding the Boca virus NP1 and NS2 polypeptides comprises SEQ ID NO: 12. In some embodiments, the Boca virus NP1 and NS2 polypeptides comprise an amino acid sequence having at least 80%, at least 85%, at least 90%, at least 95%, at least 97%, at least 98%, at least 99% or 100% identity to SEQ ID NO: 52. In some embodiments, the Boca virus NP1 and NS2 polypeptides comprise an amino acid sequence having at least 90% identity to SEQ ID NO: 52. In some embodiments, the Boca virus NP1 and NS2 polypeptides comprise an amino acid sequence having at least 95% identity to SEQ ID NO: 52. In some embodiments, the Boca virus NP1 and NS2 polypeptides comprise an amino acid sequence having at least 98% identity to SEQ ID NO: 52. In some embodiments, the Boca virus NP1 and NS2 polypeptides comprise the amino acid sequence of SEQ ID NO: 52. In some embodiments, the nucleotide sequence encoding the Boca virus NP1 and NS2 polypeptides comprises a CMV promoter. In some embodiments, the nucleotide sequence encoding the Boca virus NP1 and NS2 polypeptides comprises an engineered CMV immediate early promoters. In some embodiments, the CMV immediate early promoter comprises a nucleotide sequence of having at least 80%, at least 85%, at least 90%, at least 95%, at least 97%, at least 98%, at least 99% or 100% identity to SEQ ID NO: 121. In some embodiments, the CMV immediate early promoter comprises the nucleotide sequence of SEQ ID NO: 121. Engineered CMV immediate early promoters or transcriptionally active fragments or portions thereof are known to one of skill, for example, as disclosed in International Application No. PCT/US2023/061014, filed Jan. 20, 2023, which is incorporated herein by reference in its entirety.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide, a nucleotide sequence encoding an adenovirus VA RNA I and VA RNA II and a Boca virus NP1 and NS2 polypeptides. In some embodiments, the fragment comprises a nucleotide sequence having at least 80%, at least 85%, at least 90%, at least 95%, at least 97%, at least 98%, at least 99% or 100% identity to SEQ ID NO: 13. In some embodiments, the fragment comprises a nucleotide sequence having at least 90% identity to SEQ ID NO: 13. In some embodiments, the fragment comprises a nucleotide sequence having at least 95% identity to SEQ ID NO: 13. In some embodiments, the fragment comprises a nucleotide sequence having at least 98% identity to SEQ ID NO: 13. In some embodiments, the fragment comprises the nucleotide sequence of SEQ ID NO: 13.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide, a nucleotide sequence encoding an adenovirus VA RNA I and VA RNA II and a Boca virus NP1 and NS2 polypeptides. In some embodiments, the fragment comprises a nucleotide sequence having at least 80%, at least 85%, at least 90%, at least 95%, at least 97%, at least 98%, at least 99% or 100% identity to SEQ ID NO: 14. In some embodiments, the fragment comprises a nucleotide sequence having at least 90% identity to SEQ ID NO: 14. In some embodiments, the fragment comprises a nucleotide sequence having at least 95% identity to SEQ ID NO: 14. In some embodiments, the fragment comprises a nucleotide sequence having at least 98% identity to SEQ ID NO: 14. In some embodiments, the fragment comprises the nucleotide sequence of SEQ ID NO: 14.

In some embodiments, an isolated recombinant polynucleotide described herein comprises a fragment comprising a nucleotide sequence encoding an adenovirus E2A DBP, a nucleotide sequence encoding an adenovirus E4 ORF6, a nucleotide sequence encoding an adenovirus VA RNA I and VA RNA II and a nucleotide sequence encoding a Boca virus NP1 and NS2.

### Adeno-Associated Virus Assembly-Activating Protein

In some embodiments, an isolated recombinant polynucleotide described herein further comprises a nucleotide sequence encoding an adeno-associated virus (AAV) assembly-activating protein (AAP). A skilled artisan understands that the AAV AAP ORF overlaps with the AAV capsid ORF in the wild type virus, and consequently there are AAV serotype specific AAPs, e.g., AAP 1 to 13 corresponding to AAV serotypes 1 to 13. Sonntag et al., Journal of Virology, 85:12686-12697 (2011). In some embodiments the AAP is AAP 1, AAP 2, AAP 3B, AAP 4, AAP 5, AAP 6, AAP 7, AAP 8, AAP 9, AAP 10, AAP 11, AAP 12 or AAV 13. In some embodiments, the AAP isotype matches the capsid isotype of the recombinant AAV being produced. In some embodiments, the AAP is AAP 8. In some embodiments, the AAP is AAP 9. In some embodiments, the AAP comprises an amino acid sequence having at least 80%, at least 85%, at least 90%, at least 95%, at least 97%, at least 98%, at least 99% or 100% identity to SEQ ID NO: 53. In some embodiments, the AAP comprises the amino acid sequence of SEQ ID NO: 53. In some embodiments, the nucleotide sequence encoding the AAV AAP has at least 80%, at least 85%, at least 90%, at least 95%, at least 97%, at least 98%, at least 99% or 100% identity to SEQ ID NO: 15. In some embodiments, the nucleotide sequence encoding the AAV AAP has at least 90% identity to SEQ ID NO: 15. In some embodiments, the nucleotide sequence encoding the AAV AAP has at least 95% identity to SEQ ID NO: 15. In some embodiments, the nucleotide sequence encoding the AAV AAP has at least 98% identity to SEQ ID NO: 15. In some embodiments, the nucleotide sequence encoding the AAV AAP comprises SEQ ID NO: 15. In some embodiments, the AAV AAP comprises an amino acid sequence having at least 80%, at least 85%, at least 90%, at least 95%, at least 97%, at least 98%, at least 99% or 100% identity to SEQ ID NO: 53. In some embodiments, the AAV AAP comprises an amino acid sequence having at least 90% identity to SEQ ID NO: 53. In some embodiments, the AAV AAP comprises an amino acid sequence having at least 95% identity to SEQ ID NO: 53. In some embodiments, the AAV AAP comprises an amino acid sequence having at least 98% identity to SEQ ID NO: 53. In some embodiments, the AAV AAP comprises the amino acid sequence of SEQ ID NO: 53. In some embodiments, the nucleotide sequence encoding the AAV AAP comprises a CMV promoter. In some embodiments, the nucleotide sequence encoding the AAV AAP comprises an engineered CMV immediate early promoters. In some embodiments, the CMV immediate early promoter comprises a nucleotide sequence of having at least 80%, at least 85%, at least 90%, at least 95%, at least 97%, at least 98%, at least 99% or 100% identity to SEQ ID NO: 121. In some embodiments, the CMV immediate early promoter comprises the nucleotide sequence of SEQ ID NO: 121. Engineered CMV immediate early promoters or transcriptionally active fragments or portions thereof are known to one of skill, for example, as disclosed in International Application No. PCT/US2023/061014, filed Jan. 20, 2023, which is incorporated herein by reference in its entirety.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide, a nucleotide sequence encoding an adenovirus VA RNA I and VA RNA II and an adeno-associated virus (AAV) assembly-activating protein (AAP). In some embodiments, the fragment comprises a nucleotide sequence having at least 80%, at least 85%, at least 90%, at least 95%, at least 97%, at least 98%, at least 99% or 100% identity to SEQ ID NO: 16. In some embodiments, the fragment comprises a nucleotide sequence having at least 90% identity to SEQ ID NO: 16. In some embodiments, the fragment comprises a nucleotide sequence having at least 95% identity to SEQ ID NO: 16. In some embodiments, the fragment comprises a nucleotide sequence having at least 98% identity to SEQ ID NO: 16. In some embodiments, the fragment comprises the nucleotide sequence of SEQ ID NO: 16.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide, a nucleotide sequence encoding an adenovirus VA RNA I and VA RNA II and an adeno-associated virus (AAV) assembly-activating protein (AAP). In some embodiments, the fragment comprises a nucleotide sequence having at least 80%, at least 85%, at least 90%, at least 95%, at least 97%, at least 98%, at least 99% or 100% identity to SEQ ID NO: 17. In some embodiments, the fragment comprises a nucleotide sequence having at least 90% identity to SEQ ID NO: 17. In some embodiments, the fragment comprises a nucleotide sequence having at least 95% identity to SEQ ID NO: 17. In some embodiments, the fragment comprises a nucleotide sequence having at least 98% identity to SEQ ID NO: 17. In some embodiments, the fragment comprises the nucleotide sequence of SEQ ID NO: 17.

In some embodiments, an isolated recombinant polynucleotide described herein comprises a fragment comprising a nucleotide sequence encoding an adenovirus E2A DBP, a nucleotide sequence encoding an adenovirus E4 ORF6, a nucleotide sequence encoding an adenovirus VA RNA I and VA RNA II and a nucleotide sequence encoding an adeno-associated virus (AAV) assembly-activating protein (AAP).

In some embodiments, an isolated recombinant polynucleotide described herein comprising a nucleotide sequence encoding an adenovirus E2A DBP, a nucleotide sequence encoding an adenovirus E4 polypeptide and a nucleotide sequence encoding an adenovirus VA RNA I, and optionally VA RNA II, further comprises a nucleotide sequence encoding membrane-associated assembly protein (mAAP). A skilled artisan understands that the mAAP ORF overlaps with the VP1 AAV capsid ORF in the wild type virus, and consequently there are AAV serotype specific mAAPs. Overexpression of mAAP can increase yield of packaged viral particles. See, e.g., International Publication Nos. WO2021/226253, WO2021260204 and WO2022046998, each of which is incorporated herein by reference in its entirety. In some embodiments, the mAAP isotype matches the capsid isotype of the recombinant AAV being produced.

Adenovirus E1A polypeptide

In some embodiments, an isolated recombinant polynucleotide described herein further comprises a nucleotide sequence encoding an adenovirus E1A polypeptide. In some embodiments, the nucleotide sequence encoding the adenovirus E1A polypeptide has at least 80%, at least 85%, at least 90%, at least 95%, at least 97%, at least 98%, at least 99% or 100% identity to SEQ ID NO: 18. In some embodiments, the nucleotide sequence encoding the adenovirus E1A polypeptide has at least 90% identity to SEQ ID NO: 18. In some embodiments, the nucleotide sequence encoding the adenovirus E1A polypeptide has at least 95% identity to SEQ ID NO: 18. In some embodiments, the nucleotide sequence encoding the adenovirus E1A polypeptide has at least 98% identity to SEQ ID NO: 18. In some embodiments, the nucleotide sequence encoding the adenovirus E1A polypeptide comprises SEQ ID NO: 18. In some embodiments, the adenovirus E1A polypeptide comprises an amino acid sequence having at least 80%, at least 85%, at least 90%, at least 95%, at least 97%, at least 98%, at least 99% or 100% identity to SEQ ID NO: 51. In some embodiments, the adenovirus E1A polypeptide comprises an amino acid sequence having at least 90% identity to SEQ ID NO: 51. In some embodiments, the adenovirus E1A polypeptide comprises an amino acid sequence having at least 95% identity to SEQ ID NO: 51. In some embodiments, the adenovirus E1A polypeptide comprises an amino acid sequence having at least 98% identity to SEQ ID NO: 51. In some embodiments, the adenovirus E1A polypeptide comprises the amino acid sequence of SEQ ID NO: 51. In some embodiments, the nucleotide sequence encoding the adenovirus E1A polypeptide comprises a CMV promoter. In some embodiments, the nucleotide sequence encoding the adenovirus E1A polypeptide comprises an engineered CMV immediate early promoters. In some embodiments, the CMV immediate early promoter comprises a nucleotide sequence of having at least 80%, at least 85%, at least 90%, at least 95%, at least 97%, at least 98%, at least 99% or 100% identity to SEQ ID NO: 121. In some embodiments, the CMV immediate early promoter comprises the nucleotide sequence of SEQ ID NO: 121. Engineered CMV immediate early promoters or transcriptionally active fragments or portions thereof are known to one of skill, for example, as disclosed in International Application No. PCT/US2023/061014, filed Jan. 20, 2023, which is incorporated herein by reference in its entirety.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide, a nucleotide sequence encoding an adenovirus VA RNA I and VA RNA II and an adenovirus E1A polypeptide. In some embodiments, the fragment comprises a nucleotide sequence having at least 80%, at least 85%, at least 90%, at least 95%, at least 97%, at least 98%, at least 99% or 100% identity to SEQ ID NO: 19. In some embodiments, the fragment comprises a nucleotide sequence having at least 90% identity to SEQ ID NO: 19. In some embodiments, the fragment comprises a nucleotide sequence having at least 95% identity to SEQ ID NO: 19. In some embodiments, the fragment comprises a nucleotide sequence having at least 98% identity to SEQ ID NO: 19. In some embodiments, the fragment comprises the nucleotide sequence of SEQ ID NO: 19.

In some embodiments, an isolated recombinant polynucleotide described herein comprises a fragment comprising a nucleotide sequence encoding an adenovirus E2A DBP, a nucleotide sequence encoding an adenovirus E4 ORF6 and ORF7 polypeptide, a nucleotide sequence encoding an adenovirus VA RNA I and VA RNA II and an adenovirus E1A polypeptide. In some embodiments, the fragment comprises a nucleotide sequence having at least 80%, at least 85%, at least 90%, at least 95%, at least 97%, at least 98%, at least 99% or 100% identity to SEQ ID NO: 20. In some embodiments, the fragment comprises a nucleotide sequence having at least 90% identity to SEQ ID NO: 20. In some embodiments, the fragment comprises a nucleotide sequence having at least 95% identity to SEQ ID NO: 20. In some embodiments, the fragment comprises a nucleotide sequence having at least 98% identity to SEQ ID NO: 20. In some embodiments, the fragment comprises the nucleotide sequence of SEQ ID NO: 20.

In some embodiments, an isolated recombinant polynucleotide described herein comprises a fragment comprising a nucleotide sequence encoding an adenovirus E2A DBP, a nucleotide sequence encoding an adenovirus E4 ORF6, a nucleotide sequence encoding an adenovirus VA RNA I and VA RNA II and a nucleotide sequence encoding an adenovirus E1A polypeptide.

### Plasmids

In some embodiments, the disclosure provides a plasmid comprising a recombinant polynucleotide described herein wherein the plasmid encodes one or more helper functions, an AAV rep gene and an AAV cap gene, wherein the plasmid is capable of promoting production of recombinant AAV particles in a host cell, e.g., an HEK cell. In some embodiments, a plasmid described herein comprises a recombinant polynucleotide comprising (a) a nucleotide sequence encoding an adenovirus E2A DNA binding protein (DBP); (b) a nucleotide sequence encoding an adenovirus E4 polypeptide; (c) a nucleotide sequence encoding an adenovirus VA RNA I, and (d) a nucleotide sequence encoding an AAV rep gene and an AAV cap gene. In some embodiments, the adenovirus E4 polypeptide comprises E4 ORF6 and ORF7. In some embodiments, the adenovirus E4 polypeptide comprises the E4 ORF6. In some embodiments, the nucleotide sequence encoding an adenovirus VA RNA I encodes an adenovirus VA RNA I and VA RNA II. In some embodiments, the nucleotide sequence encoding an AAV rep gene and an AAV cap gene comprises a nucleotide sequence encoding a parvovirus p5 promoter and a nucleotide sequence encoding an AAV rep gene and an AAV cap gene, wherein the parvovirus p5 promoter is operably linked to the AAV rep gene and controls the expression of the rep78 and rep68 gene products. In some embodiments, the nucleotide sequence encoding a parvovirus p5 promoter is immediately upstream of the nucleotide sequence encoding an AAV rep gene. In some embodiments, the nucleotide sequence encoding a parvovirus p5 promoter is separated from the nucleotide sequence encoding an AAV rep gene by between about 1 and about 10,000 nucleotides. In some embodiments, the plasmid does not comprise a nucleotide sequence encoding an adenovirus ITR sequence, L3 23K endoprotease, L5 pVI/fibre, and/or L4 pVIII/hexon-associated precursor. In some embodiments, the nucleotide sequence encoding the adenovirus ITR sequence, L3 23K endoprotease, L5 pVI/fibre, and/or L4 pVIII/hexon-associated precursor is the corresponding nucleotide sequence of pAdDeltaF6. In some embodiments, the plasmid is a bacterial plasmid.

In some embodiments, the disclosure provides a plasmid comprising a recombinant polynucleotide described herein wherein the plasmid encodes one or more helper functions, an AAV rep gene, an AAV cap gene, and a recombinant AAV viral genome, wherein the plasmid is capable of promoting production of recombinant AAV particles in a host cell, e.g., an HEK cell. In some embodiments, a plasmid described herein comprises a recombinant polynucleotide comprising (a) a nucleotide sequence encoding an adenovirus E2A DNA binding protein (DBP); (b) a nucleotide sequence encoding an adenovirus E4 polypeptide; (c) a nucleotide sequence encoding an adenovirus VA RNA I, (d) a nucleotide sequence encoding an AAV rep gene and an AAV cap gene, and (e) a nucleotide sequence encoding a recombinant viral genome comprising at least one AAV inverted terminal repeat (ITR) and a non-AAV nucleic acid sequence encoding a gene product operably linked to sequences which direct expression of the gene product in a target cell. In some embodiments, the adenovirus E4 polypeptide comprises E4 ORF6 and ORF7. In some embodiments, the adenovirus E4 polypeptide comprises the E4 ORF6. In some embodiments, the nucleotide sequence encoding an adenovirus VA RNA I encodes an adenovirus VA RNA I and VA RNA II. In some embodiments, the nucleotide sequence encoding an AAV rep gene and an AAV cap gene comprises a nucleotide sequence encoding a parvovirus p5 promoter and a nucleotide sequence encoding an AAV rep gene and an AAV cap gene, wherein the parvovirus p5 promoter is operably linked to the AAV rep gene and controls the expression of the rep78 and rep68 gene products. In some embodiments, the nucleotide sequence encoding a parvovirus p5 promoter is immediately upstream of the nucleotide sequence encoding an AAV rep gene. In some embodiments, the nucleotide sequence encoding a parvovirus p5 promoter is separated from the nucleotide sequence encoding an AAV rep gene by between about 1 and about 10,000 nucleotides. In some embodiments, the plasmid does not comprise a nucleotide sequence encoding an adenovirus ITR sequence, L3 23K endoprotease, L5 pVI/fibre, and/or L4 pVIII/hexon-associated precursor. In some embodiments, the nucleotide sequence encoding the adenovirus ITR sequence, L3 23K endoprotease, L5 pVI/fibre, and/or L4 pVIII/hexon-associated precursor is the corresponding nucleotide sequence of pAdDeltaF6. In some embodiments, the plasmid is a bacterial plasmid.

In some embodiments, a plasmid described herein comprises a bacterial replication origin capable of propagating the plasmid in a bacterial host cell, e.g., E. coli host cell. In some embodiments, the bacterial replication origin is a ColE1 origin.

In some embodiments, a plasmid described herein comprises a selectable marker gene. In some embodiments, the selectable marker gene is a drug resistance gene. In some embodiments, the selectable marker gene is a kanamycin resistance gene. In some embodiments, the selectable marker gene is an ampicillin resistance gene.

In some embodiments, a plasmid described herein comprises a bacterial replication origin and a selectable marker gene.

In some embodiments, a plasmid described herein comprises (a) a nucleotide sequence encoding an adenovirus E2A DNA binding protein (DBP); (b) a nucleotide sequence encoding an adenovirus E4 polypeptide; (c) a nucleotide sequence encoding an adenovirus VA RNA I, (d) a nucleotide sequence encoding an AAV rep gene and an AAV cap gene, (e) a nucleotide sequence encoding a parvovirus p5 promoter, and (f) a nucleotide sequence encoding a replication origin and a selectable marker gene, wherein the plasmid comprises the nucleotide sequences in the order of 5′-(d)-(a)-(b)-(c)-(f)-3′, wherein the parvovirus p5 promoter is operably linked to the AAV rep gene and controls the expression of the rep78 and rep68 gene products. In some embodiments, the adenovirus E4 polypeptide comprises E4 ORF6 and ORF7. In some embodiments, the adenovirus E4 polypeptide comprises the E4 ORF6. In some embodiments, the nucleotide sequence encoding a parvovirus p5 promoter is immediately upstream of the nucleotide sequence encoding an AAV rep gene. In some embodiments, the nucleotide sequence encoding an adenovirus VA RNA I encodes an adenovirus VA RNA I and VA RNA II. In some embodiments, the nucleotide sequence encoding a parvovirus p5 promoter is separated from the nucleotide sequence encoding an AAV rep gene by between about 1 and about 10,000 nucleotides. In some embodiments, the p5 promoter is positioned between about 10 and about 10,000 nucleotides upstream from the AAV rep start codon. In some embodiments, the p5 promoter is positioned between about 1,000 and about 4,000 nucleotides upstream from the AAV rep start codon. In some embodiments, the p5 promoter is positioned between about 1,000 and about 2,000 nucleotides upstream from the AAV rep start codon. In some embodiments, the p5 promoter is positioned between about 2,000 and about 3,000 nucleotides upstream from the AAV rep start codon. In some embodiments, the p5 promoter is positioned between about 3,000 and about 4,000 nucleotides upstream from the AAV rep start codon. In some embodiments, the p5 promoter is positioned between nucleotide sequences (f) and (d). In some embodiments, the p5 promoter is positioned between nucleotide sequences (c) and (f). In some embodiments, the p5 promoter is positioned between nucleotide sequences (b) and (c). In some embodiments, the p5 promoter is positioned between the replication origin and the selectable marker gene. In some embodiments, the adenovirus E2A DBP coding region and the adenovirus E4 polypeptide coding region are in opposite 5′ to 3′ orientation. In some embodiments, the adenovirus E2A DBP coding region and the AAV rep gene and AAV cap gene coding regions are in opposite 5′ to 3′ orientation. In some embodiments, the adenovirus E2A DBP coding region and the adenovirus E4 polypeptide coding region are in opposite 5′ to 3′ orientation and the adenovirus E2A DBP coding region and the AAV rep gene and AAV cap gene coding regions are in opposite 5′ to 3′ orientation. In some embodiments, the plasmid does not comprise a nucleotide sequence encoding an adenovirus ITR sequence, L3 23K endoprotease, L5 pVI/fibre, and/or L4 pVIII/hexon-associated precursor. In some embodiments, the plasmid comprises between about 10,000 and 15,000 nucleotides. In some embodiments, the plasmid comprises between about 10,000 and 14,000 nucleotides. In some embodiments, the plasmid comprises between about 10,000 and 13,000 nucleotides. In some embodiments, the plasmid comprises between about 12,000 and 13,000 nucleotides.

In some embodiments, a plasmid described herein comprises a nucleotide sequence having at least 80%, at least 85%, at least 90%, at least 95%, at least 97%, at least 98%, or at least 99% identity to any one of the pHRC #1 to #39 packaging plasmid.

In some embodiments, a plasmid described herein comprises the nucleotide sequence of any one of the pHRC #1 to #39 packaging plasmid.

In some embodiments, a plasmid described herein comprises the nucleotide sequence of the pHRC #5 or #7 packaging plasmid.

In some embodiments, a plasmid described herein comprises the nucleotide sequence of the pHRC #35, #36 or #37 packaging plasmid.

In some embodiments, a plasmid described herein comprises the nucleotide sequence of any one of the pHRC #8 to #16 packaging plasmid.

In some embodiments, a plasmid described herein comprises a nucleotide sequence having at least 80%, at least 85%, at least 90%, at least 95%, at least 97%, at least 98%, or at least 99% identity to SEQ ID NO: 72-86 or 118.

In some embodiments, a plasmid described herein comprises the nucleotide sequence of SEQ ID NO: 72-86 or 118.

In some embodiments, a plasmid described herein comprises a nucleotide sequence having at least 80%, at least 85%, at least 90%, at least 95%, at least 97%, at least 98%, or at least 99% identity to SEQ ID NO: 72-86, 118, 159-161, 168, 173, 178, 183, 188, 193, 198, 203, 208, 213, 218, 223, 228, 233, 238, 243, 248, 253, 558, or 263.

In some embodiments, a plasmid described herein comprises a nucleotide sequence having at least 80%, at least 85%, at least 90%, at least 95%, at least 97%, at least 98%, or at least 99% identity to SEQ ID NO: 76 or 77.

In some embodiments, a plasmid described herein comprises a nucleotide sequence having at least 80%, at least 85%, at least 90%, at least 95%, at least 97%, at least 98%, or at least 99% identity to SEQ ID NO: 78-85 or 86.

In some embodiments, a plasmid described herein comprises a nucleotide sequence having at least 80%, at least 85%, at least 90%, at least 95%, at least 97%, at least 98%, or at least 99% identity to SEQ ID NO: 159, 160 or 161.

In some embodiments, a plasmid described herein comprises the nucleotide sequence of SEQ ID NO: 87-101, 119, 162-164, 169, 174, 179, 184, 189, 194, 199, 204, 209, 214, 219, 224, 229, 234, 239, 244, 249, 254, 259, or 264.

In some embodiments, a plasmid described herein comprises the nucleotide sequence of SEQ ID NO: 91 or 92.

In some embodiments, a plasmid described herein comprises the nucleotide sequence of SEQ ID NO: 93-100 or 101.

In some embodiments, a plasmid described herein comprises the nucleotide sequence of SEQ ID NO: 162, 163 or 164.

In some embodiments, a plasmid described herein comprises a nucleotide sequence having at least 80%, at least 85%, at least 90%, at least 95%, at least 97%, at least 98%, or at least 99% identity to SEQ ID NO: 10, 11, 25-34, 56, 57, 106-108 or 109.

In some embodiments, a plasmid described herein comprises the nucleotide sequence of SEQ ID NO: 10, 11, 25-34, 56, 57, 106-108 or 109.

In some embodiments, a plasmid described herein comprises a nucleotide sequence having at least 80%, at least 85%, at least 90%, at least 95%, at least 97%, at least 98%, or at least 99% identity to SEQ ID NO: 110-116 or 117.

In some embodiments, a plasmid described herein comprises the nucleotide sequence of SEQ ID NO: 110-116 or 117.

In some embodiments, a plasmid described herein comprises (a) a nucleotide sequence encoding an adenovirus E2A DNA binding protein (DBP); (b) a nucleotide sequence encoding an adenovirus E4 polypeptide; (c) a nucleotide sequence encoding an adenovirus VA RNA I, (d) a nucleotide sequence encoding an AAV rep gene and an AAV cap gene, (e) a nucleotide sequence encoding a parvovirus p5 promoter, (f) a nucleotide sequence encoding a recombinant AAV viral genome comprising a nucleic acid sequence encoding a gene product, and (g) a nucleotide sequence encoding a replication origin and a selectable marker gene, wherein the plasmid comprises the nucleotide sequences in the order of 5′-(d)-(a)-(b)-(f)-(c)-(g)-3′, wherein the parvovirus p5 promoter is operably linked to the AAV rep gene and controls the expression of the rep78 and rep68 gene products. In some embodiments, the adenovirus E4 polypeptide comprises E4 ORF6 and ORF7. In some embodiments, the adenovirus E4 polypeptide comprises the E4 ORF6. In some embodiments, the nucleotide sequence encoding an adenovirus VA RNA I encodes an adenovirus VA RNA I and VA RNA II. In some embodiments, the nucleotide sequence encoding a parvovirus p5 promoter is immediately upstream of the nucleotide sequence encoding an AAV rep gene. In some embodiments, the nucleotide sequence encoding a parvovirus p5 promoter is separated from the nucleotide sequence encoding an AAV rep gene by between about 1 and about 10,000 nucleotides. In some embodiments, the p5 promoter is positioned between about 10 and about 10,000 nucleotides upstream from the AAV rep start codon. In some embodiments, the p5 promoter is positioned between about 1,000 and about 4,000 nucleotides upstream from the AAV rep start codon. In some embodiments, the p5 promoter is positioned between about 1,000 and about 2,000 nucleotides upstream from the AAV rep start codon. In some embodiments, the p5 promoter is positioned between about 2,000 and about 3,000 nucleotides upstream from the AAV rep start codon. In some embodiments, the p5 promoter is positioned between about 3,000 and about 4,000 nucleotides upstream from the AAV rep start codon. In some embodiments, the p5 promoter is positioned between nucleotide sequences (g) and (d). In some embodiments, the p5 promoter is positioned between nucleotide sequences (c) and (g). In some embodiments, the p5 promoter is positioned between nucleotide sequences (f) and (c). In some embodiments, the p5 promoter is positioned between the replication origin and the selectable marker gene. In some embodiments, the adenovirus E2A DBP coding region and the adenovirus E4 polypeptide coding region are in opposite 5′ to 3′ orientation. In some embodiments, the adenovirus E2A DBP coding region and the AAV rep gene and AAV cap gene coding regions are in opposite 5′ to 3′ orientation. In some embodiments, the adenovirus E2A DBP coding region and the adenovirus E4 polypeptide coding region are in opposite 5′ to 3′ orientation and the adenovirus E2A DBP coding region and the AAV rep gene and AAV cap gene coding regions are in opposite 5′ to 3′ orientation. In some embodiments, the AAV rep gene and AAV cap gene coding regions and the gene product coding region comprised by the recombinant AAV viral genome are in opposite 5′ to 3′ orientation. In some embodiments, the plasmid does not comprise a nucleotide sequence encoding an adenovirus ITR sequence, L3 23K endoprotease, L5 pVI/fibre, and/or L4 pVIII/hexon-associated precursor. In some embodiments, the plasmid comprises between about 12,000 and 17,000 nucleotides. In some embodiments, the plasmid comprises between about 12,000 and 16,000 nucleotides. In some embodiments, the plasmid comprises between about 12,000 and 15,000 nucleotides. In some embodiments, the plasmid comprises between about 13,000 and 15,000 nucleotides.

In some embodiments, a plasmid described herein comprises a nucleotide sequence having at least 80%, at least 85%, at least 90%, at least 95%, at least 97%, at least 98%, or at least 99% identity to the pHRCG #1, #2, #3 or #4 complete plasmid excluding the nucleotide sequence encoding a recombinant AAV viral genome comprising a nucleic acid sequence encoding a gene product. In some embodiments, the nucleotide sequence encoding a recombinant AAV viral genome within pHRCG #1, #2, #3 or #4 spans the regions encoding the 5′ and 3′ ITRs and the sequence between the 5′ and 3′ ITRs.

In some embodiments, the plasmid comprises the pHRCG #1, #2, #3 or #4 plasmid excluding the nucleotide sequence encoding a recombinant AAV viral genome comprising a nucleic acid sequence encoding a gene product. In some embodiments, the nucleotide sequence encoding a recombinant AAV viral genome within pHRCG #1, #2, #3 or #4 spans the regions encoding the 5′ and 3′ ITRs and the sequence between the 5′ and 3′ ITRs.

In some embodiments, a plasmid described herein comprises a nucleotide sequence having at least 80%, at least 85%, at least 90%, at least 95%, at least 97%, at least 98%, or at least 99% identity to SEQ ID NO: 102, 103, 267 or 268 excluding the nucleotide sequence encoding a recombinant AAV viral genome comprising a nucleic acid sequence encoding a gene product. In some embodiments, the nucleotide sequence encoding a recombinant AAV viral genome spans the regions encoding the 5′ and 3′ ITRs and the sequence between the 5′ and 3′ ITRs.

In some embodiments, a plasmid described herein comprises the nucleotide sequence of SEQ ID NO: 102, 103, 267 or 268 excluding the nucleotide sequence encoding a recombinant AAV viral genome comprising a nucleic acid sequence encoding a gene product. In some embodiments, the nucleotide sequence encoding a recombinant AAV viral genome spans the regions encoding the 5′ and 3′ ITRs and the sequence between the 5′ and 3′ ITRs.

In some embodiments, a plasmid described herein comprises a nucleotide sequence having at least 80%, at least 85%, at least 90%, at least 95%, at least 97%, at least 98%, or at least 99% identity to SEQ ID NO: 104, 105, 269 or 270 excluding the nucleotide sequence encoding a recombinant AAV viral genome comprising a nucleic acid sequence encoding a gene product. In some embodiments, the nucleotide sequence encoding a recombinant AAV viral genome spans the regions encoding the 5′ and 3′ ITRs and the sequence between the 5′ and 3′ ITRs. In some embodiments, the nucleotide sequence encoding a recombinant AAV viral genome comprises the nucleotide sequence of residues 9,537-14,270 of SEQ ID NO: 104 or residues 9,526-14,259 of SEQ ID NO: 105.

In some embodiments, a plasmid described herein comprises the nucleotide sequence of SEQ ID NO: 104, 105, 269 or 270 excluding the nucleotide sequence encoding a recombinant AAV viral genome comprising a nucleic acid sequence encoding a gene product. In some embodiments, the nucleotide sequence encoding a recombinant AAV viral genome spans the regions encoding the 5′ and 3′ ITRs and the sequence between the 5′ and 3′ ITRs. In some embodiments, the nucleotide sequence encoding a recombinant AAV viral genome comprises the nucleotide sequence of residues 9,537-14,270 of SEQ ID NO: 104 or residues 9,526-14,259 of SEQ ID NO: 105.

In some embodiments, a plasmid described herein comprises a nucleotide sequence having at least 80%, at least 85%, at least 90%, at least 95%, at least 97%, at least 98%, or at least 99% identity to SEQ ID NO: 110-116 or 117.

In some embodiments, a plasmid described herein comprises the nucleotide sequence of SEQ ID NO: 110-116 or 117.

In some embodiments, a plasmid described herein comprises a nucleotide sequence with at least about 80%, at least about 85%, at least about 90%, at least about 95%, at least about 97%, at least about 98%, at least about 99% or 100% identity to a nucleotide sequence shown in Table 1. In some embodiments, a plasmid described herein comprises a nucleotide sequence shown in Table 1.

TABLE 1
SEQ ID Nos for plasmid sequences (N/A not applicable)

| | E2A/E4/VA | | | w/o | |
|---|---|---|---|---|---|
| | helper | Cap | | plasmid | Full |
| | function | gene | Rep/Cap | backbone | plasmid |
| helper | | N/A | N/A | | 35 |
| plasmids #1 | | | | | |
| helper | | N/A | N/A | | 36 |
| plasmids #2 | | | | | |
| helper | 10 & 11 | N/A | N/A | N/A | 37 |
| plasmids #3 | | | | | |
| helper | 122 & 123 | N/A | N/A | N/A | 132 |
| plasmids #3A | | | | | |
| helper | 124 & 125 | N/A | N/A | N/A | 133 |
| plasmids #3B | | | | | |
| helper | 126 & 127 | N/A | N/A | N/A | 134 |
| plasmids #3C | | | | | |
| helper | 128 & 129 | N/A | N/A | N/A | 135 |
| plasmids #3D | | | | | |
| helper | 56 & 57 | N/A | N/A | N/A | 38 |
| plasmids #4 | | | | | |
| helper | 25 & 26 | N/A | N/A | N/A | 39 |
| plasmids #5 | | | | | |
| helper | 130 & 131 | N/A | N/A | N/A | 136 |
| plasmids #5A | | | | | |
| helper | 27 & 28 | N/A | N/A | N/A | 40 |
| plasmids #6 | | | | | |
| helper | 29 & 30 | N/A | N/A | N/A | 41 |
| plasmids #7 | | | | | |
| helper | 31 & 32 | N/A | N/A | N/A | 42 |
| plasmids #8 | | | | | |
| helper | 33 & 34 | N/A | N/A | N/A | 43 |
| plasmids #9 | | | | | |
| helper | 137 & 138 | N/A | N/A | N/A | 139 |
| plasmids #10 | | | | | |
| pHRC #1 | 140 | 66 | 70 | 72 | 87 |
| pHRC #2 | 141 | 66 | 70 | 73 | 88 |
| pHRC #3 | 142 | 66 | 70 | 74 | 89 |
| pHRC #4 | 143 | 66 | 70 | 75 | 90 |
| pHRC #5 | 144 | 66 | 70 | 76 | 91 |
| pHRC #7 | 145 | 66 | 70 | 77 | 92 |
| pHRC #6 | 283 | 66 | 70 | 118 | 119 |
| pHRC #8 | 146 | 67 | 71 | 78 | 93 |
| pHRC #9 | 147 | 64 | 68 | 79 | 94 |
| pHRC #10 | 148 | 65 | 69 | 80 | 95 |
| pHRC #11 | 149 | 67 | 71 | 81 | 96 |
| pHRC #12 | 150 | 64 | 68 | 82 | 97 |
| pHRC #13 | 151 | 65 | 69 | 83 | 98 |
| pHRC #14 | 152 | 67 | 71 | 84 | 99 |
| pHRC #15 | 153 | 64 | 68 | 85 | 100 |
| pHRC #16 | 154 | 65 | 69 | 86 | 101 |
| pHRC #17 | 165 | 166 | 167 | 168 | 169 |
| pHRC #18 | 170 | 171 | 172 | 173 | 174 |
| pHRC #19 | 175 | 176 | 177 | 178 | 179 |
| pHRC #20 | 180 | 181 | 182 | 183 | 184 |
| pHRC #21 | 185 | 186 | 187 | 188 | 189 |
| pHRC #22 | 190 | 191 | 192 | 193 | 194 |
| pHRC #23 | 195 | 196 | 197 | 198 | 199 |
| pHRC #24 | 200 | 201 | 202 | 203 | 204 |
| pHRC #25 | 205 | 206 | 207 | 208 | 209 |
| pHRC #26 | 210 | 211 | 212 | 213 | 214 |
| pHRC #27 | 215 | 216 | 217 | 218 | 219 |
| pHRC #28 | 220 | 221 | 222 | 223 | 224 |
| pHRC #29 | 225 | 226 | 227 | 228 | 229 |
| pHRC #30 | 230 | 231 | 232 | 233 | 234 |
| pHRC #31 | 235 | 236 | 237 | 238 | 239 |
| pHRC #32 | 240 | 241 | 242 | 243 | 244 |
| pHRC #33 | 245 | 246 | 247 | 248 | 249 |
| pHRC #34 | 250 | 251 | 252 | 253 | 254 |
| pHRC #35 | 156 | 66 | 70 | 159 | 162 |
| pHRC #36 | 157 | 66 | 70 | 160 | 163 |
| pHRC #37 | 158 | 66 | 70 | 161 | 164 |
| pHRC #38 | 255 | 256 | 257 | 258 | 259 |
| pHRC #39 | 260 | 261 | 262 | 263 | 264 |
| pHRCG #1 | 271 | 66 | 70 | 102 | 104 |
| pHRCG #2 | 272 | 66 | 70 | 103 | 105 |
| pHRCG #3 | 265 | 66 | 70 | 267 | 269 |
| pHRCG #4 | 266 | 66 | 70 | 268 | 270 |

In some embodiments, a helper plasmid described herein comprises a nucleotide sequence having at least 80%, at least 85%, at least 90%, at least 95%, at least 97%, at least 98%, at least 99% or 100% identity to E2A/E4/VA helper function sequence shown in Table 1. In some embodiments, a helper plasmid described herein comprises a nucleotide sequence having the sequence of a E2A/E4/VA helper function sequence shown in Table 1.

In some embodiments, a helper plasmid described herein comprises a nucleotide sequence having at least 80%, at least 85%, at least 90%, at least 95%, at least 97%, at least 98%, at least 99% or 100% identity to a helper plasmid “full plasmid” sequence shown in Table 1. In some embodiments, a helper plasmid described herein comprises a nucleotide sequence having the sequence of a helper plasmid “full plasmid” sequence shown in Table 1.

In some embodiments, a packaging plasmid described herein comprises a nucleotide sequence having at least 80%, at least 85%, at least 90%, at least 95%, at least 97%, at least 98%, at least 99% or 100% identity to E2A/E4/VA helper function sequence shown in Table 1, a nucleotide sequence encoding a rep gene and a nucleotide sequence encoding a cap gene. In some embodiments, the packaging plasmid further comprises a nucleotide sequence encoding a p5 promoter. In some embodiments, the packaging plasmid comprises a nucleotide sequence having the sequence of a E2A/E4/VA helper function sequence shown in Table 1. In some embodiments, the nucleotide sequence encoding a cap gene comprises a nucleotide sequence having at least 80%, at least 85%, at least 90%, at least 95%, at least 97%, at least 98%, at least 99% or 100% identity to a cap gene sequence shown in Table 1. In some embodiments, the nucleotide sequence encoding a cap gene comprises a cap gene sequence shown in Table 1. In some embodiments, the nucleotide sequence encoding the rep gene and the nucleotide sequence encoding the cap gene comprises a nucleotide sequence having at least 80%, at least 85%, at least 90%, at least 95%, at least 97%, at least 98%, at least 99% or 100% identity to a rep/cap sequence shown in Table 1. In some embodiments, the nucleotide sequence encoding the rep gene and the nucleotide sequence encoding the cap gene comprises a rep/cap sequence shown in Table 1.

In some embodiments, a packaging plasmid described herein comprises a nucleotide sequence having at least 80%, at least 85%, at least 90%, at least 95%, at least 97%, at least 98%, at least 99% or 100% identity to a pHRC “full plasmid” sequence shown in Table 1. In some embodiments, a packaging plasmid described herein comprises a nucleotide sequence having the sequence of a pHCR “full plasmid” sequence shown in Table 1.

In some embodiments, a plasmid described herein is less than 15,000 bp long. In some embodiments, a plasmid described herein is less than 13,000 bp long. In some embodiments, a plasmid described herein is between 10,000 and 15,000 bp long.

### Host Cells

In some embodiments, the disclosure provides a host cell comprising a recombinant polynucleotide or a plasmid described herein. In some embodiments, the host cell is a prokaryotic cell capable of propagating a recombinant polynucleotide or a plasmid described herein. In some embodiments, the prokaryotic host cell is a bacterial cell. In some embodiments, the prokaryotic host cell is E. coli. In some embodiments, the host cell is a eukaryotic cell capable of producing recombinant AAV particles. In some embodiments, the eukaryotic host cell is a mammalian cell. In some embodiments, the eukaryotic host cell is a HEK293 cell, HEK derived cell, CHO cell, CHO derived cell, HeLa cell, SF-9 cell, BHK cell, Vero cell, or PerC6 cell.

In some embodiments, a host cell described herein comprises a recombinant polynucleotide encoding one or more helper functions, and an AAV rep gene and an AAV cap gene, wherein the polynucleotide is capable of promoting production of recombinant AAV particles in a host cell, e.g., an HEK cell. In some embodiments, the host cell comprises an isolated recombinant polynucleotide comprising (a) a nucleotide sequence encoding an adenovirus E2A DNA binding protein (DBP); (b) a nucleotide sequence encoding an adenovirus E4 polypeptide; (c) a nucleotide sequence encoding an adenovirus VA RNA I, and (d) a nucleotide sequence encoding an AAV rep gene and an AAV cap gene. In some embodiments, the adenovirus E4 polypeptide comprises E4 ORF6 and ORF7. In some embodiments, the adenovirus E4 polypeptide comprises the E4 ORF6. In some embodiments, the nucleotide sequence encoding an adenovirus VA RNA I encodes an adenovirus VA RNA I and VA RNA II. In some embodiments, the nucleotide sequence encoding an AAV rep gene and an AAV cap gene comprises a nucleotide sequence encoding a parvovirus p5 promoter and a nucleotide sequence encoding an AAV rep gene and an AAV cap gene, wherein the parvovirus p5 promoter is operably linked to the AAV rep gene and controls the expression of the rep78 and rep68 gene products. In some embodiments, the recombinant polynucleotide does not comprise a nucleotide sequence encoding an adenovirus ITR sequence, L3 23K endoprotease, L5 pVI/fibre, and/or L4 pVIII/hexon-associated precursor. In some embodiments, the plasmid is a bacterial plasmid.

In some embodiments, a host cell described herein comprises a recombinant polynucleotide encoding one or more helper functions, an AAV rep gene and an AAV cap gene, and a recombinant AAV viral genome comprising a nucleic acid sequence encoding a gene product, wherein the polynucleotide is capable of promoting production of recombinant AAV particles in a host cell, e.g., an HEK cell. In some embodiments, the host cell comprises an isolated recombinant polynucleotide comprising (a) a nucleotide sequence encoding an adenovirus E2A DBP, (b) a nucleotide sequence encoding an adenovirus E4 polypeptide, (c) a nucleotide sequence encoding an adenovirus VA RNA I, (d) a nucleotide sequence encoding an AAV rep gene and an AAV cap gene, and (e) a nucleotide sequence encoding a recombinant AAV viral genome comprising a nucleic acid sequence encoding a gene product. In some embodiments, the adenovirus E4 polypeptide comprises E4 ORF6 and ORF7. In some embodiments, the adenovirus E4 polypeptide comprises the E4 ORF6. In some embodiments, the nucleotide sequence encoding an adenovirus VA RNA I encodes an adenovirus VA RNA I and VA RNA II. In some embodiments, the nucleotide sequence encoding an AAV rep gene and an AAV cap gene comprises a nucleotide sequence encoding a parvovirus p5 promoter and a nucleotide sequence encoding an AAV rep gene and an AAV cap gene, wherein the parvovirus p5 promoter is operably linked to the AAV rep gene and controls the expression of the rep78 and rep68 gene products. In some embodiments, the recombinant polynucleotide does not comprise a nucleotide sequence encoding an adenovirus ITR sequence, L3 23K endoprotease, L5 pVI/fibre, and/or L4 pVIII/hexon-associated precursor. In some embodiments, the plasmid is a bacterial plasmid.

In some embodiments, a host cell described herein comprises a plasmid described herein comprising (a) a nucleotide sequence encoding an adenovirus E2A DNA binding protein (DBP); (b) a nucleotide sequence encoding an adenovirus E4 polypeptide; (c) a nucleotide sequence encoding an adenovirus VA RNA I, (d) a nucleotide sequence encoding an AAV rep gene and an AAV cap gene, (e) a nucleotide sequence encoding a parvovirus p5 promoter, and (f) a nucleotide sequence encoding a replication origin and a selectable marker gene, wherein the plasmid comprises the nucleotide sequences in the order of 5′-(d)-(a)-(b)-(c)-(f)-3′, wherein the parvovirus p5 promoter is operably linked to the AAV rep gene and controls the expression of the rep78 and rep68 gene products. In some embodiments, the adenovirus E4 polypeptide comprises E4 ORF6 and ORF7. In some embodiments, the adenovirus E4 polypeptide comprises the E4 ORF6. In some embodiments, the nucleotide sequence encoding an adenovirus VA RNA I encodes an adenovirus VA RNA I and VA RNA II. In some embodiments, the plasmid does not comprise a nucleotide sequence encoding an adenovirus ITR sequence, L3 23K endoprotease, L5 pVI/fibre, and/or L4 pVIII/hexon-associated precursor. In some embodiments, the plasmid is a bacterial plasmid.

In some embodiments, a host cell described herein comprises a plasmid described herein comprising (a) a nucleotide sequence encoding an adenovirus E2A DNA binding protein (DBP); (b) a nucleotide sequence encoding an adenovirus E4 polypeptide; (c) a nucleotide sequence encoding an adenovirus VA RNA I, (d) a nucleotide sequence encoding an AAV rep gene and an AAV cap gene, (e) a nucleotide sequence encoding a parvovirus p5 promoter, (f) a nucleotide sequence encoding a recombinant AAV viral genome comprising a nucleic acid sequence encoding a gene product, and (g) a nucleotide sequence encoding a replication origin and a selectable marker gene, wherein the plasmid comprises the nucleotide sequences in the order of 5′-(d)-(a)-(b)-(f)-(c)-(g)-3′, wherein the parvovirus p5 promoter is operably linked to the AAV rep gene and controls the expression of the rep78 and rep68 gene products. In some embodiments, the adenovirus E4 polypeptide comprises E4 ORF6 and ORF7. In some embodiments, the adenovirus E4 polypeptide comprises the E4 ORF6. In some embodiments, the nucleotide sequence encoding an adenovirus VA RNA I encodes an adenovirus VA RNA I and VA RNA II. In some embodiments, the plasmid does not comprise a nucleotide sequence encoding an adenovirus ITR sequence, L3 23K endoprotease, L5 pVI/fibre, and/or L4 pVIII/hexon-associated precursor. In some embodiments, the plasmid is a bacterial plasmid.

In some embodiments, the disclosure provides a method of producing a recombinant polynucleotide described herein or a plasmid described herein comprising incubating a host cell described herein under suitable conditions to produce the recombinant polynucleotide or a plasmid. In some embodiments, the host cell is a prokaryotic cell capable of propagating a plasmid described herein. In some embodiments, the prokaryotic host cell is a bacterial cell. In some embodiments, the prokaryotic host cell is E. coli.

### Methods of Producing a Recombinant Viral Particle

In one aspect, the disclosure provides a method of producing recombinant adeno-associated virus (rAAV) particles in a eukaryotic host cell by using a recombinant polynucleotide, plasmid or host cell described herein. In some embodiments, the method further comprises recovering the rAAV particles.

In some embodiments, the disclosure provides a method of producing recombinant adeno-associated virus (rAAV) particles comprising culturing a cell capable of producing the rAAV particles, wherein the cell comprises (i) a polynucleotide encoding an AAV capsid protein; (ii) a polynucleotide encoding a functional rep gene; (iii) a polynucleotide comprising a genome comprising at least one AAV inverted terminal repeat (ITR) and a non-AAV nucleic acid sequence encoding a gene product operably linked to sequences which direct expression of the gene product in a target cell; and (iv) one or more polynucleotides comprising sufficient helper functions to permit packaging of the genome into the AAV capsid protein under conditions which permit packaging of the genome into the AAV capsid, wherein the recombinant polynucleotide described herein or a plasmid described herein comprise a recombinant polynucleotide encoding one or more helper functions, an AAV rep gene and an AAV cap gene, and optionally a recombinant AAV viral genome comprising a nucleic acid sequence encoding a gene product. In some embodiments, the one or more helper functions comprise a nucleotide sequence encoding the adenovirus E2A DBP, a nucleotide sequence encoding the adenovirus E4 polypeptide and a nucleotide sequence encoding the adenovirus VA RNA I. In some embodiments, the adenovirus E4 polypeptide comprises E4 ORF6 and ORF7. In some embodiments, the adenovirus E4 polypeptide comprises the E4 ORF6. In some embodiments, the nucleotide sequence encoding an adenovirus VA RNA I encodes an adenovirus VA RNA I and VA RNA II. In some embodiments, the method further comprises recovering the rAAV particles. In some embodiments, the cell comprises (i) one polynucleotide disclosed herein comprising (a) a nucleotide sequence encoding an adenovirus E2A DNA binding protein (DBP); (b) a nucleotide sequence encoding an adenovirus E4 polypeptide; (c) a nucleotide sequence encoding an adenovirus VA RNA I, and (d) a nucleotide sequence encoding an AAV rep gene and an AAV cap gene and (ii) one polynucleotide encoding the rAAV genome to be packaged. In some embodiments, the adenovirus E4 polypeptide comprises E4 ORF6 and ORF7. In some embodiments, the adenovirus E4 polypeptide comprises the E4 ORF6. In some embodiments, the cell comprises one polynucleotide disclosed herein comprising (a) a nucleotide sequence encoding an adenovirus E2A DBP, (b) a nucleotide sequence encoding an adenovirus E4 polypeptide, (c) a nucleotide sequence encoding an adenovirus VA RNA I, (d) a nucleotide sequence encoding an AAV rep gene and an AAV cap gene, and (e) a nucleotide sequence encoding a recombinant AAV viral genome comprising a nucleic acid sequence encoding a gene product. In some embodiments, the adenovirus E4 polypeptide comprises E4 ORF6 and ORF7. In some embodiments, the adenovirus E4 polypeptide comprises the E4 ORF6. In some embodiments, the rAAV particles are AAV8 or AAV9 particles. In some embodiments, the rAAV particles have an AAV capsid protein of a serotype selected from the group consisting of AAV.rh8, AAV.rh10, AAV.rh20, AAV.rh39, AAV.Rh74, AAV.RHM4-1, AAV.hu32, AAV.hu37, AAV.PHB, and AAV.7m8. In some embodiments, the rAAV particles have an AAV capsid protein with high sequence homology to AAV8 or AAV9 such as, AAV.rh10, AAV.rh20, AAV.rh39, AAV.Rh74, AAV.RHM4-1, AAV.hu32, and AAV.hu37. In some embodiments, the cell culture is a suspension culture. In some embodiments, the cell culture comprises HEK293 cells adapted for growth in suspension culture. In some embodiments, the cell culture has a volume of between about 400 liters and about 5,000 liters.

In some embodiments, the disclosure provides a method of producing recombinant adeno-associated virus (rAAV) particles comprising (a) providing a cell culture comprising a cell; (b) introducing into the cell a first polynucelotide disclosed here comprising (i) a nucleotide sequence encoding an adenovirus E2A DNA binding protein (DBP); (ii) a nucleotide sequence encoding an adenovirus E4 polypeptide; (iii) a nucleotide sequence encoding an adenovirus VA RNA I, and (iv) a nucleotide sequence encoding an AAV rep gene and an AAV cap gene and a second polynucleotide comprising a genome comprising at least one AAV inverted terminal repeat (ITR) and a non-AAV nucleic acid sequence encoding a gene product operably linked to sequences which direct expression of the gene product in a target cell, and (c) maintaining the cell culture under conditions that allow production of the rAAV particles. In some embodiments, the adenovirus E4 polypeptide comprises E4 ORF6 and ORF7. In some embodiments, the adenovirus E4 polypeptide comprises the E4 ORF6. In some embodiments, the disclosure provides a method of producing recombinant adeno-associated virus (rAAV) particles comprising (a) providing a cell culture comprising a cell; (b) introducing into the cell a plasmid disclosed here comprising (i) a nucleotide sequence encoding an adenovirus E2A DNA binding protein (DBP); (ii) a nucleotide sequence encoding an adenovirus E4 polypeptide; (iii) a nucleotide sequence encoding an adenovirus VA RNA I, and (iv) a nucleotide sequence encoding an AAV rep gene and an AAV cap gene and a polynucleotide comprising a genome comprising at least one AAV inverted terminal repeat (ITR) and a non-AAV nucleic acid sequence encoding a gene product operably linked to sequences which direct expression of the gene product in a target cell, and (c) maintaining the cell culture under conditions that allow production of the rAAV particles. In some embodiments, the adenovirus E4 polypeptide comprises E4 ORF6 and ORF7. In some embodiments, the adenovirus E4 polypeptide comprises the E4 ORF6. In some embodiments, the nucleotide sequence encoding an adenovirus VA RNA I encodes an adenovirus VA RNA I and VA RNA II. In some embodiments, the method further comprises recovering the rAAV particles. In some embodiments, the rAAV particles are AAV8 or AAV9 particles. In some embodiments, the rAAV particles have an AAV capsid protein of a serotype selected from the group consisting of AAV.rh8, AAV.rh10, AAV.rh20, AAV.rh39, AAV.Rh74, AAV.RHM4-1, AAV.hu32, AAV.hu37, AAV.PHB, and AAV.7m8. In some embodiments, the rAAV particles have an AAV capsid protein with high sequence homology to AAV8 or AAV9 such as, AAV.rh10, AAV.rh20, AAV.rh39, AAV.Rh74, AAV.RHM4-1, AAV.hu32, and AAV.hu37. In some embodiments, the cell culture is a suspension culture. In some embodiments, the cell culture comprises HEK293 cells adapted for growth in suspension culture. In some embodiments, the cell culture has a volume of between about 400 liters and about 5,000 liters.

In some embodiments, the disclosure provides a method of producing recombinant adeno-associated virus (rAAV) particles comprising (a) providing a cell culture comprising a cell; (b) introducing into the cell a polynucelotide disclosed here comprising (i) a nucleotide sequence encoding an adenovirus E2A DBP, (ii) a nucleotide sequence encoding an adenovirus E4 polypeptide, (iii) a nucleotide sequence encoding an adenovirus VA RNA I, (iv) a nucleotide sequence encoding an AAV rep gene and an AAV cap gene, and (v) a nucleotide sequence encoding a recombinant AAV viral genome comprising a nucleic acid sequence encoding a gene product, and (c) maintaining the cell culture under conditions that allow production of the rAAV particles. In some embodiments, the adenovirus E4 polypeptide comprises E4 ORF6 and ORF7. In some embodiments, the adenovirus E4 polypeptide comprises the E4 ORF6. In some embodiments, the disclosure provides a method of producing recombinant adeno-associated virus (rAAV) particles comprising (a) providing a cell culture comprising a cell; (b) introducing into the cell a plasmid disclosed here comprising (i) a nucleotide sequence encoding an adenovirus E2A DBP, (ii) a nucleotide sequence encoding an adenovirus E4 polypeptide, (iii) a nucleotide sequence encoding an adenovirus VA RNA I, (iv) a nucleotide sequence encoding an AAV rep gene and an AAV cap gene, and (v) a nucleotide sequence encoding a recombinant AAV viral genome comprising a nucleic acid sequence encoding a gene product, and (c) maintaining the cell culture under conditions that allow production of the rAAV particles. In some embodiments, the adenovirus E4 polypeptide comprises E4 ORF6 and ORF7. In some embodiments, the adenovirus E4 polypeptide comprises the E4 ORF6. In some embodiments, the nucleotide sequence encoding an adenovirus VA RNA I encodes an adenovirus VA RNA I and VA RNA II. In some embodiments, the method further comprises recovering the rAAV particles. In some embodiments, the rAAV particles are AAV8 or AAV9 particles. In some embodiments, the rAAV particles have an AAV capsid protein of a serotype selected from the group consisting of AAV.rh8, AAV.rh10, AAV.rh20, AAV.rh39, AAV.Rh74, AAV.RHM4-1, AAV.hu32, AAV.hu37, AAV.PHB, and AAV.7m8. In some embodiments, the rAAV particles have an AAV capsid protein with high sequence homology to AAV8 or AAV9 such as, AAV.rh10, AAV.rh20, AAV.rh39, AAV.Rh74, AAV.RHM4-1, AAV.hu32, and AAV.hu37. In some embodiments, the cell culture is a suspension culture. In some embodiments, the cell culture comprises HEK293 cells adapted for growth in suspension culture. In some embodiments, the cell culture has a volume of between about 400 liters and about 5,000 liters.

In some embodiments, a method disclosed herein comprises introducing into the cell a polynucleotide encoding an AAV capsid protein and a functional rep gene.

In some embodiments, the introducing of the one or more polynucleotides and/or the one or more plasmids into the cell is by transfection.

In some embodiments, the cell is a mammalian cell. In some embodiments, the cell is an insect cell. In some embodiments, the cell is a HEK293 cell, HEK derived cell, CHO cell, CHO derived cell, HeLa cell, SF-9 cell, BHK cell, Vero cell, or PerC6 cell. In some embodiments, the cell is a HEK293 cell.

In some embodiments, the cell culture is a suspension culture or an adherent culture. In some embodiments, the cell culture is a suspension culture.

In some embodiments, the cell culture has a volume between about 50 liters and about 20,000 liters.

In some embodiments, a method described herein produces more rAAV particles measured as GC/ml than a reference method. In some embodiments, the reference method uses a polynucleotide comprising helper functions that comprises the nucleotide sequence of SEQ ID NO: 35. In some embodiments, the reference method uses a polynucleotide comprising helper functions that comprises the nucleotide sequence of SEQ ID NO: 44. In some embodiments, the method described herein produces at least about 10% more rAAV particles measured as GC/ml than the reference method. In some embodiments, the method described herein produces at least about 10% more rAAV particles measured as GC/ml than the reference method. In some embodiments, the method described herein produces at least about 20% more rAAV particles measured as GC/ml than the reference method. In some embodiments, the method described herein produces at least about 30% more rAAV particles measured as GC/ml than the reference method. In some embodiments, the method described herein produces at least about 40% more rAAV particles measured as GC/ml than the reference method. In some embodiments, the method described herein produces at least about 50% more rAAV particles measured as GC/ml than the reference method. In some embodiments, the method described herein produces at least about 70% more rAAV particles measured as GC/ml than the reference method. In some embodiments, the method described herein produces at least about 90% more rAAV particles measured as GC/ml than the reference method. In some embodiments, the method described herein produces at least about twice as many rAAV particles measured as GC/ml than the reference method. In some embodiments, the method produces at least about three times as many rAAV particles measured as GC/ml than the reference method. In some embodiments, the method produces at least about four times as many rAAV particles measured as GC/ml than the reference method.

In some embodiments, the method produces a population of rAAV particles comprising more full capsids than a reference method. In some embodiments, the reference method uses a polynucleotide comprising helper functions that comprises the nucleotide sequence of SEQ ID NO: 35. In some embodiments, the reference method uses a polynucleotide comprising helper functions that comprises the nucleotide sequence of SEQ ID NO: 44.

In some embodiments, the rAAV particles comprise a capsid protein of the AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 serotype. In some embodiments, the rAAV particles comprise a capsid protein of the AAV8, AAV9, AAV.rh10, AAV.rh20, AAV.rh39, AAV.Rh74, AAV.RHM4-1, AAV.hu32, or AAV.hu37 serotype. In some embodiments, the rAAV particles comprise a capsid protein of the AAV8 serotype. In some embodiments, the rAAV particles comprise a capsid protein of the AAV9 serotype.

In some embodiments, the rAAV particle comprises a transgene encoding a gene product. In some embodiments, the gene product is a polypeptide or a double stranded RNA molecule. In some embodiments, the gene product is a polypeptide. In some embodiments, the transgene encodes an antibody or antigen-binding fragment thereof, fusion protein, Fc-fusion polypeptide, immunoadhesin, immunoglobulin, engineered protein, protein fragment or enzyme. In some embodiments, the transgene comprises a regulatory element operatively connected to a polynucleotide encoding the gene product.

In some embodiments, the gene product is anti-VEGF Fab, anti-kallikrein antibody, anti-TNF antibody, microdystrophin, minidystrophin, iduronidase (IDUA), iduronate 2-sulfatase (IDS), low-density lipoprotein receptor (LDLR), tripeptidyl peptidase 1 (TPP1), or non-membrane associated splice variant of VEGF receptor 1 (sFlt-1). In some embodiments, the gene product is an gamma-sarcoglycan, Rab Escort Protein 1 (REP1/CHM), retinoid isomerohydrolase (RPE65), cyclic nucleotide gated channel alpha 3 (CNGA3), cyclic nucleotide gated channel beta 3 (CNGB3), aromatic L-amino acid decarboxylase (AADC), lysosome-associated membrane protein 2 isoform B (LAMP2B), Factor VIII, Factor IX, retinitis pigmentosa GTPase regulator (RPGR), retinoschisin (RS1), sarcoplasmic reticulum calcium ATPase (SERCA2a), aflibercept, battenin (CLN3), transmembrane ER protein (CLN6), glutamic acid decarboxylase (GAD), Glial cell line-derived neurotrophic factor (GDNF), aquaporin 1 (AQP1), dystrophin, myotubularin 1 (MTM1), follistatin (FST), glucose-6-phosphatase (G6Pase), apolipoprotein A2 (APOA2), uridine diphosphate glucuronosyl transferase 1A1 (UGT1A1), arylsulfatase B (ARSB), N-acetyl-alpha-glucosaminidase (NAGLU), alpha-glucosidase (GAA), alpha-galactosidase (GLA), beta-galactosidase (GLB1), lipoprotein lipase (LPL), alpha 1-antitrypsin (AAT), phosphodiesterase 6B (PDE6B), ornithine carbamoyltransferase 90TC), survival motor neuron (SMN1), survival motor neuron (SMN2), neurturin (NRTN), Neurotrophin-3 (NT-3/NTF3), porphobilinogen deaminase (PBGD), nerve growth factor (NGF), mitochondrially encoded NADH: ubiquinone oxidoreductase core subunit 4 (MT-ND4), protective protein cathepsin A (PPCA), dysferlin, MER proto-oncogene, tyrosine kinase (MERTK), cystic fibrosis transmembrane conductance regulator (CFTR), or tumor necrosis factor receptor (TNFR)-immunoglobulin (IgG1) Fc fusion. In some embodiments, the gene product is a dystrophin or a microdystrophin. In some embodiments, the gene product is a microRNA.

In some embodiments, a method described herein increases production of rAAV particles while maintaining or improving the quality attributes of the rAAV particles and compositions comprising thereof. In some embodiments, the quality of rAAV particles and compositions comprising thereof is assessed by determining the concentration of rAAV particles (e.g., GC/m1), the percentage of particles comprising a copy of the rAAV genome; the ratio of particles without a genome, infectivity of the rAAV particles, stability of rAAV particles, concentration of residual host cell proteins, or concentration of residual host cell nucleic acids (e.g., host cell genomic DNA, plasmid encoding rep and cap genes, plasmid encoding helper functions, plasmid encoding rAAV genome). In some embodiments, the quality of rAAV particles produced by a method described herein or compositions comprising thereof is the same as that of rAAV particles or compositions produced by a reference method using a helper plasmid comprising the nucleotide sequence of SEQ ID NO: 35 or 44. In some embodiments, the quality of rAAV particles produced by a method described herein or compositions comprising thereof is better than the quality of rAAV particles or compositions produced by a reference method using a helper plasmid comprising the nucleotide sequence of SEQ ID NO: 35 or 44.

Numerous cell culture based systems are known in the art for production of rAAV particles, any of which can be used to practice a method described herein. rAAV production cultures for the production of rAAV virus particles require; (1) suitable host cells, including, for example, human-derived cell lines such as HeLa, A549, or HEK293 cells and their derivatives (HEK293T cells, HEK293F cells), or mammalian cell lines such as Vero, CHO cells or CHO-derived cells; (2) suitable helper virus function, provided by wild type or mutant adenovirus (such as temperature sensitive adenovirus), herpes virus, baculovirus, or a plasmid construct providing helper functions; (3) AAV rep and cap genes and gene products; (4) a transgene (such as a therapeutic transgene) flanked by AAV ITR sequences; and (5) suitable media and media components to support rAAV production.

A skilled artisan is aware of the numerous methods by which AAV rep and cap genes, AAV helper genes (e.g., adenovirus E1a gene, E1b gene, E4 gene, E2a gene, and VA gene), and rAAV genomes (comprising one or more genes of interest flanked by inverted terminal repeats (ITRs)) can be introduced into cells to produce or package rAAV. The phrase “adenovirus helper functions” refers to a number of viral helper genes expressed in a cell (as RNA or protein) such that the AAV grows efficiently in the cell. The skilled artisan understands that helper viruses, including adenovirus and herpes simplex virus (HSV), promote AAV replication and certain genes have been identified that provide the essential functions, e.g., the helper may induce changes to the cellular environment that facilitate such AAV gene expression and replication. In some embodiments of a method described herein, AAV rep and cap genes, helper genes, and rAAV genomes are introduced into cells by transfection of one or more plasmid vectors encoding the AAV rep and cap genes, helper genes, and rAAV genome.

Molecular biology techniques to develop plasmid or viral vectors encoding the AAV rep and cap genes, helper genes, and/or rAAV genome are commonly known in the art. In some embodiments, AAV rep and cap genes are encoded by one plasmid vector. In some embodiments, AAV helper genes (e.g., adenovirus E1a gene, E1b gene, E4 gene, E2a gene, and VA gene) are encoded by one plasmid vector. In some embodiments, the E1a gene or E1b gene is stably expressed by the host cell, and the remaining AAV helper genes are introduced into the cell by transfection by one viral vector. In some embodiments, the E1a gene and E1b gene are stably expressed by the host cell, and the E4 gene, E2a gene, and VA gene are introduced into the cell by transfection by one plasmid vector. In some embodiments, one or more helper genes are stably expressed by the host cell, and one or more helper genes are introduced into the cell by transfection by one plasmid vector. In some embodiments, the helper genes are stably expressed by the host cell. In some embodiments, AAV rep and cap genes are encoded by one viral vector. In some embodiments, AAV helper genes (e.g., adenovirus E1a gene, E1b gene, E4 gene, E2a gene, and VA gene) are encoded by one viral vector. In some embodiments, the E1a gene or E1b gene is stably expressed by the host cell, and the remaining AAV helper genes are introduced into the cell by transfection by one viral vector. In some embodiments, the E1a gene and E1b gene are stably expressed by the host cell, and the E4 gene, E2a gene, and VA gene are introduced into the cell by transfection by one viral vector. In some embodiments, one or more helper genes are stably expressed by the host cell, and one or more helper genes are introduced into the cell by transfection by one viral vector. In some embodiments, the AAV rep and cap genes, the adenovirus helper functions necessary for packaging, and the rAAV genome to be packaged are introduced to the cells by transfection with one or more polynucleotides, e.g., vectors. In some embodiments, a method described herein comprises transfecting the cells with a mixture of three polynucleotides: one encoding the cap and rep genes, one encoding adenovirus helper functions necessary for packaging (e.g., adenovirus E1a gene, E1b gene, E4 gene, E2a gene, and VA gene), and one encoding the rAAV genome to be packaged. In some embodiments, the AAV cap gene is an AAV8 or AAV9 cap gene. In some embodiments, the AAV cap gene is an AAV.rh8, AAV.rh10, AAV.rh20, AAV.rh39, AAV.Rh74, AAV.RHM4-1, AAV.hu32, AAV.hu37, AAV.PHB, or AAV.7m8 cap gene. In some embodiments, the AAV cap gene encodes a capsid protein with high sequence homology to AAV8 or AAV9 such as, AAV.rh10, AAV.rh20, AAV.rh39, AAV.Rh74, AAV.RHM4-1, AAV.hu32, and AAV.hu37. In some embodiments, the vector encoding the rAAV genome to be packaged comprises a gene of interest flanked by AAV ITRs. In some embodiments, the AAV ITRs are from AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other AAV serotype.

Any combination of vectors can be used to introduce AAV rep and cap genes, AAV helper genes, and rAAV genome to a cell in which rAAV particles are to be produced or packaged. In some embodiments of a method described herein, a first plasmid vector encoding an rAAV genome comprising a gene of interest flanked by AAV inverted terminal repeats (ITRs), a second vector encoding AAV rep and cap genes, and a third vector encoding helper genes can be used. In some embodiments, a mixture of the three vectors is co-transfected into a cell.

In some embodiments, a combination of transfection and infection is used by using both plasmid vectors as well as viral vectors.

In some embodiments, one or more of rep and cap genes, and AAV helper genes are constitutively expressed by the cells and does not need to be transfected or transduced into the cells. In some embodiments, the cell constitutively expresses rep and/or cap genes. In some embodiments, the cell constitutively expresses one or more AAV helper genes. In some embodiments, the cell constitutively expresses Ela. In some embodiments, the cell comprises a stable transgene encoding the rAAV genome.

In some embodiments, AAV rep, cap, and helper genes (e.g., E1a gene, E1b gene, E4 gene, E2a gene, or VA gene) can be of any AAV serotype. Similarly, AAV ITRs can also be of any AAV serotype. For example, in some embodiments, AAV ITRs are from AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other AAV serotypes (e.g., a hybrid serotype harboring sequences from more than one serotype). In some embodiments, AAV cap gene is from AAV9 or AAV8 cap gene. In some embodiments, an AAV cap gene is from AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other AAV serotypes (e.g., a hybrid serotype harboring sequences from more than one serotype). In some embodiments, AAV rep and cap genes for the production of a rAAV particle are from different serotypes. For example, the rep gene is from AAV2 whereas the cap gene is from AAV9.

Any suitable media known in the art can be used for the production of recombinant virus particles (e.g., rAAV particles) according to a method described herein. These media include, without limitation, media produced by Hyclone Laboratories and JRH including Modified Eagle Medium (MEM), Dulbecco's Modified Eagle Medium (DMEM), and Sf-900 II SFM media as described in U.S. Pat. No. 6,723,551, which is incorporated herein by reference in its entirety. In some embodiments, the medium comprises Dynamis™ Medium, FreeStyle™ 293 Expression Medium, or Expi293™ Expression Medium from Invitrogen/ThermoFisher. In some embodiments, the medium comprises Dynamis™ Medium. In some embodiments, a method described herein uses a cell culture comprising a scrum-free medium, an animal-component free medium, or a chemically defined medium. In some embodiments, the medium is an animal-component free medium. In some embodiments, the medium comprises serum. In some embodiments, the medium comprises fetal bovine serum. In some embodiments, the medium is a glutamine-free medium. In some embodiments, the medium comprises glutamine. In some embodiments, the medium is supplemented with one or more of nutrients, salts, buffering agents, and additives (e.g., antifoam agent). In some embodiments, the medium is supplemented with glutamine. In some embodiments, the medium is supplemented with serum. In some embodiments, the medium is supplemented with fetal bovine serum. In some embodiments, the medium is supplemented with poloxamer, e.g., Kolliphor® P 188 Bio. In some embodiments, a medium is a base medium. In some embodiments, the medium is a feed medium.

Recombinant virus (e.g., rAAV) production cultures can routinely be grown under a variety of conditions (over a wide temperature range, for varying lengths of time, and the like) suitable to the particular host cell being utilized. As is known in the art, virus production cultures include suspension-adapted host cells such as HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, RK cells, Per.C6 cells, chicken embryo cells and SF-9 cells which can be cultured in a variety of ways including, for example, spinner flasks, stirred tank bioreactors, and disposable systems such as the Wave bag system. Numerous suspension cultures are known in the art for production of rAAV particles, including for example, the cultures disclosed in U.S. Pat. Nos. 6,995,006, 9,783,826, and in U.S. Pat. Appl. Pub. No. 20120122155, each of which is incorporated herein by reference in its entirety.

Any cell or cell line that is known in the art to produce recombinant virus particles (e.g., rAAV particles) can be used in any one of the methods described herein. In some embodiments, a method of producing recombinant virus particles (e.g., rAAV particles) or increasing the production of recombinant virus particles (e.g., a rAAV particles) described herein uses HeLa cells, HEK293 cells, HEK293 derived cells (e.g., HEK293T cells, HEK293F cells), Vero cells, CHO cells, CHO-K1 cells, CHO derived cells, EB66 cells, LLC-MK cells, MDCK cells, RAF cells, RK cells, TCMK-1 cells, PK15 cells, BHK cells, BHK-21 cells, NS-1 cells, BHK cells, 293 cells, RK cells, Per.C6 cells, chicken embryo cells or SF-9 cells. In some embodiments, a method described herein uses mammalian cells. In some embodiments, a method described herein uses insect cells, e.g., SF-9 cells. In some embodiments, a method described herein uses cells adapted for growth in suspension culture. In some embodiments, a method described herein uses HEK293 cells adapted for growth in suspension culture.

In some embodiments, a cell culture described herein is a suspension culture. In some embodiments, a large scale suspension cell culture described herein comprises HEK293 cells adapted for growth in suspension culture. In some embodiments, a cell culture described herein comprises a serum-free medium, an animal-component free medium, or a chemically defined medium. In some embodiments, a cell culture described herein comprises a serum-free medium. In some embodiments, suspension-adapted cells are cultured in a shaker flask, a spinner flask, a cell bag, or a bioreactor.

In some embodiments, a cell culture described herein comprises a serum-free medium, an animal-component free medium, or a chemically defined medium. In some embodiments, a cell culture described herein comprises a serum-free medium.

In some embodiments, a large scale suspension culture cell culture described herein comprises a high density cell culture. In some embodiments, the culture has a total cell density of between about 1×10E+06 cells/ml and about 30×10E+06 cells/ml. In some embodiments, more than about 50% of the cells are viable cells. In some embodiments, the cells are HeLa cells, HEK293 cells, HEK293 derived cells (e.g., HEK293T cells, HEK293F cells), Vero cells, or SF-9 cells. In further embodiments, the cells are HEK293 cells.

Methods described herein can be used in the production of rAAV particles comprising a capsid protein from any AAV capsid serotype. In some embodiments, the rAAV particles comprise a capsid protein from an AAV capsid serotype selected from AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise a capsid protein that is a derivative, modification, or pseudotype of AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 capsid protein.

In some embodiments, the rAAV particles comprise a capsid protein from an AAV capsid serotype selected from AAV8 and AAV9. In some embodiments, the rAAV particles have an AAV capsid serotype of AAV8. In some embodiments, the rAAV particles have an AAV capsid serotype of AAV9.

In some embodiments, the rAAV particles comprise a capsid protein from an AAV capsid serotype selected from the group consisting of AAV.rh8, AAV.rh10, AAV.rh20, AAV.rh39, AAV.Rh74, AAV.RHM4-1, AAV.hu32, AAV.hu37, AAV.PHB, and AAV.7m8. In some embodiments, the rAAV particles comprise a capsid protein with high sequence homology to AAV8 or AAV9 such as, AAV.rh10, AAV.rh20, AAV.rh39, AAV.Rh74, AAV.RHM4-1, AAV.hu32, and AAV.hu37.

In some embodiments, the rAAV particles comprise a capsid protein that is a derivative, modification, or pseudotype of AAV8 or AAV9 capsid protein. In some embodiments, the rAAV particles comprise a capsid protein that has an AAV8 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV8 capsid protein.

In some embodiments, the rAAV particles comprise a capsid protein that is a derivative, modification, or pseudotype of AAV9 capsid protein. In some embodiments, rAAV particles comprise a capsid protein that has an AAV9 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV9 capsid protein.

In some embodiments, the rAAV particles comprise a capsid protein that has at least 80% or more identity, e.g., 85%, 85%, 87%, 88%, 89%, 90%, 91%, 92%, 93%, 94%, 95%, 96%, 97%, 98%, 99%, 99.5%, etc., i.e. up to 100% identity, to the VP1, VP2 and/or VP3 sequence of AAV.rh8, AAV.rh10, AAV.rh20, AAV.rh39, AAV.Rh74, AAV.RHM4-1, AAV.hu32, AAV.hu37, AAV.PHB, or AAV.7m8 capsid protein. In some embodiments, the rAAV particles comprise a capsid protein that has at least 80% or more identity, e.g., 85%, 85%, 87%, 88%, 89%, 90%, 91%, 92%, 93%, 94%, 95%, 96%, 97%, 98%, 99%, 99.5%, etc., i.e. up to 100% identity, to the VP1, VP2 and/or VP3 sequence of an AAV capsid protein with high sequence homology to AAV8 or AAV9 such as, AAV.rh10, AAV.rh20, AAV.rh39, AAV.Rh74, AAV.RHM4-1, AAV.hu32, and AAV.hu37.

In additional embodiments, the rAAV particles comprise a mosaic capsid. In additional embodiments, the rAAV particles comprise a pseudotyped rAAV particle. In additional embodiments, the rAAV particles comprise a capsid containing a capsid protein chimera of two or more AAV capsid serotypes.

### RAAV Particles

The provided methods are suitable for use in the production of any isolated recombinant AAV particles. As such, the rAAV can be of any serotype, modification, or derivative, known in the art, or any combination thereof (e.g., a population of rAAV particles that comprises two or more serotypes, e.g., comprising two or more of rAAV2, rAAV8, and rAAV9 particles) known in the art. In some embodiments, the rAAV particles are AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof.

In some embodiments, rAAV particles have a capsid protein from an AAV serotype selected from AAV1, AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or a derivative, modification, or pseudotype thereof. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, rAAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise a capsid protein from an AAV capsid serotype selected from AAV1, AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise the capsid of Anc80 or Anc80L65, as described in Zinn et al., 2015, Cell Rep. 12 (6): 1056-1068, which is incorporated by reference in its entirety. In certain embodiments, the rAAV particles comprise the capsid with one of the following amino acid insertions: LGETTRP or LALGETTRP, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise the capsid of AAV.7m8, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise any AAV capsid disclosed in U.S. Pat. No. 9,585,971, such as AAVPHP.B. In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23:857-862 and Georgiadis et al., 2018, Gene Therapy 25:450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29(9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles comprise an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles have a capsid protein disclosed in Intl. Appl. Publ. No. WO 2003/052051 (see, e.g., SEQ ID NO: 2), WO 2005/033321 (see, e.g., SEQ ID NOs: 123 and 88), WO 03/042397 (see, e.g., SEQ ID NOs: 2, 81, 85, and 97), WO 2006/068888 (see, e.g., SEQ ID NOs: 1 and 3-6), WO 2006/110689, (see, e.g., SEQ ID NOs: 5-38) WO2009/104964 (see, e.g., SEQ ID NOs: 1-5, 7, 9, 20, 22, 24 and 31), WO 2010/127097 (see, e.g., SEQ ID NOs: 5-38), and WO 2015/191508 (see, e.g., SEQ ID NOs: 80-294), and U.S. Appl. Publ. No. 20150023924 (see, e.g., SEQ ID NOs: 1, 5-10), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2), WO 2005/033321 (see, e.g., SEQ ID NOs: 123 and 88), WO 03/042397 (see, e.g., SEQ ID NOs: 2, 81, 85, and 97), WO 2006/068888 (see, e.g., SEQ ID NOs: 1 and 3-6), WO 2006/110689 (see, e.g., SEQ ID NOs: 5-38) WO2009/104964 (see, e.g., SEQ ID NOs: 1-5, 7, 9, 20, 22, 24 and 31), WO 2010/127097 (see, e.g., SEQ ID NOs: 5-38), and WO 2015/191508 (see, e.g., SEQ ID NOs: 80-294), and U.S. Appl. Publ. No. 20150023924 (see, e.g., SEQ ID NOs: 1, 5-10).

Nucleic acid sequences of AAV based viral vectors and methods of making recombinant AAV and AAV capsids are taught, for example, in U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; International Patent Application Nos. PCT/US2015/034799; PCT/EP2015/053335; WO 2003/052051, WO 2005/033321, WO 03/042397, WO 2006/068888, WO 2006/110689, WO2009/104964, WO 2010/127097, and WO 2015/191508, and U.S. Appl. Publ. No. 20150023924.

The provided methods are suitable for use in the production of recombinant AAV encoding a transgene. In certain embodiments, the transgene is from Tables 2A-2C. In some embodiments, the rAAV genome comprises a vector comprising the following components: (1) AAV inverted terminal repeats that flank an expression cassette; (2) regulatory control elements, such as a) promoter/enhancers, b) a polyA signal, and c) optionally an intron; and (3) nucleic acid sequences coding for a transgene. In other embodiments for expressing an intact or substantially intact monoclonal antibody (mAb), the rAAV genome comprises a vector comprising the following components: (1) AAV inverted terminal repeats that flank an expression cassette; (2) regulatory control elements, such as a) promoter/enhancers, b) a polyA signal, and c) optionally an intron; and (3) nucleic acid sequences coding for the light chain Fab and heavy chain Fab of the antibody, or at least the heavy chain or light chain Fab, and optionally a heavy chain Fc region. In still other embodiments for expressing an intact or substantially intact mAb, the rAAV genome comprises a vector comprising the following components: (1) AAV inverted terminal repeats that flank an expression cassette; (2) regulatory control elements, such as a) promoter/enhancers, b) a polyA signal, and c) optionally an intron; and (3) nucleic acid sequences coding for the heavy chain Fab of an anti-VEGF (e.g., sevacizumab, ranibizumab, bevacizumab, and brolucizumab), anti-EpoR (e.g., LKA-651), anti-ALK1 (e.g., ascrinvacumab), anti-C5 (e.g., tesidolumab and eculizumab), anti-CD105 (e.g., carotuximab), anti-CC1Q (e.g., ANX-007), anti-TNFα (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab and sarilumab), anti-IL4R (e.g., dupilumab), anti-IL17A (e.g., ixekizumab and secukinumab), anti-IL-5 (e.g., mepolizumab), anti-IL12/IL23 (e.g., ustekinumab), anti-CD19 (e.g., inebilizumab), anti-ITGF7 mAb (e.g., etrolizumab), anti-SOST mAb (e.g., romosozumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., densomab), anti-PCSK9 (e.g., alirocumab and evolocumab), anti-ANGPTL3 (e.g., cvinacumab*), anti-OxPL (e.g., E06), anti-fD (e.g., lampalizumab), or anti-MMP9 (e.g., andecaliximab); optionally an Fc polypeptide of the same isotype as the native form of the therapeutic antibody, such as an IgG isotype amino acid sequence IgG1, IgG2 or IgG4 or modified Fc thereof; and the light chain of an anti-VEGF (e.g., sevacizumab, ranibizumab, bevacizumab, and brolucizumab), anti-EpoR (e.g., LKA-651), anti-ALK1 (e.g., ascrinvacumab), anti-C5 (e.g., tesidolumab and cculizumab), anti-CD105 or anti-ENG (e.g., carotuximab), anti-CC1Q (e.g., ANX-007), anti-TNFα (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab and sarilumab), anti-IL4R (e.g., dupilumab), anti-IL17A (e.g., ixekizumab and secukinumab), anti-IL-5 (e.g., mepolizumab), anti-IL12/IL23 (e.g., ustekinumab), anti-CD19 (e.g., inebilizumab), anti-ITGF7 mAb (e.g., etrolizumab), anti-SOST mAb (e.g., romosozumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., densomab), anti-PCSK9 (e.g., alirocumab and evolocumab), anti-ANGPTL3 (e.g., evinacumab), anti-OxPL (e.g., E06), anti-fD (e.g., lampalizumab), or anti-MMP9 (e.g., andecaliximab); wherein the heavy chain (Fab and optionally Fc region) and the light chain are separated by a self-cleaving furin (F)/F2A or flexible linker, ensuring expression of equal amounts of the heavy and the light chain polypeptides.

TABLE 2A

| Disease | Transgene |
|---|---|
| MPS I | alpha-L-iduronidase (IDUA) |
| MPS II (Hunter Syndrome) | iduronate-2-sulfatase (IDS) |
| ceroid lipofuscinosis (Batten disease) | (CLN1, CLN2, CLN10, CLN13), a soluble |
| | lysosomal protein (CLN5), a protein |
| | in the secretory pathway (CLN11), |
| | two cytoplasmic proteins that also |
| | peripherally associate with |
| | membranes (CLN4, CLN14), and many |
| | transmembrane proteins with different |
| | subcellular locations (CLN3, CLN6, CLN7, |
| | CLN8, CLN12) |
| MPS IIIa (Sanfilippo type A Syndrome) | heparan sulfate sulfatase (also called N- |
| | sulfoglucosamine sulfohydrolase (SGSH)) |
| MPS IIIB (Sanfilippo type B Syndrome) | N-acetyl-alpha-D-glucosaminidase (NAGLU) |
| MPS VI (Maroteaux-Lamy Syndrome) | arylsulfatase B |
| Gaucher disease (type 1, II and III) | Glucocerebrosidase, GBA1 |
| Parkinson's Disease | Glucocerebrosidase; GBA1 |
| Parkinson's Disease | dopamine decarboxylase |
| Pompe | acid maltase; GAA |
| Metachromatic leukodystrophy | Aryl sulfatase A |
| MPS VII (Sly syndrome) | beta-glucuronidase |
| MPS VIII | glucosamine-6-sulfate sulfatase |
| MPS IX | Hyaluronidase |
| Niemann-Pick disease | Sphingomyelinase |
| Niemann-Pick disease without | a npc1 gene encoding a |
| sphingomyelinase deficiency | cholesterol metabolizing enzyme |
| Tay-Sachs disease | Alpha subunit of beta-hexosaminidase |
| Sandhoff disease | both alpha and beta subunit of beta- |
| | hexosaminidase |
| Fabry Disease | alpha-galactosidase |
| Fucosidosis | Fucosidase (FUCA1 gene) |
| Alpha-mannosidosis | alpha-mannosidase |
| Beta-mannosidosis | Beta-mannosidase |
| Wolman disease | cholesterol ester hydrolase |
| Parkinson's disease | Neurturin |
| Parkinson's disease | glial derived growth factor (GDGF) |
| Parkinson's disease | tyrosine hydroxylase |
| Parkinson's disease | glutamic acid decarboxylase. |
| Parkinson's disease | fibroblast growth factor-2 (FGF-2) |
| Parkinson's disease | brain derived growth factor (BDGF) |
| No disease listed (Galactosialidosis | neuraminidase deficiency with |
| (Goldberg syndrome)) | betagalactosidase deficiency |
| Spinal Muscular Atrophy (SMA) | SMN |
| Friedreich's ataxia | Frataxin |
| Amyotrophic lateral sclerosis (ALS) | SOD1 |
| Glycogen Storage Disease 1a | Glucose-6-phosphatase |
| XLMTM | MTM1 |
| Crigler Najjar | UGT1A1 |
| CPVT | CASQ2 |
| Rett syndrome | MECP2 |
| Achromatopsia | CNGB3, CNGA3, GNAT2, PDE6C |
| Choroidermia | CDM |
| Danon Disease | LAMP2 |
| Cystic Fibrosis | CFTR |
| Duchenne Muscular Dystrophy | Mini-Dystrophin or Microdystrophin Gene |
| Limb Girdle Muscular Dystrophy Type | human-alpha-sarcoglycan |
| 2C\|Gamma-sarcoglycanopathy | |
| Advanced Heart Failure | SERCA2a |
| Rheumatoid Arthritis | TNFR:Fc Fusion Gene |
| Leber Congenital Amaurosis | GAA |
| Limb Girdle Muscular Dystrophy Type | gamma-sarcoglycan |
| 2C\|Gamma-sarcoglycanopathy | |
| Retinitis Pigmentosa | hMERTK |
| Age-Related Macular Degeneration | sFLT01 |
| Becker Muscular Dystrophy and | huFollistatin344 |
| Sporadic Inclusion Body Myositis | |
| Parkinson's Disease | GDNF |
| Metachromatic Leukodystrophy (MLD) | cuARSA |
| Hepatitis C | anti-HCV shRNA |
| Limb Girdle Muscular Dystrophy | hSGCA |
| Type 2D | |
| Human Immunodeficiency Virus | PG9DP |
| Infections; HIV Infections (HIV-1) | |
| Acute Intermittant Porphyria | PBGD |
| Leber's Hereditary Optical Neuropathy | P1ND4v2 |
| Alpha-1 Antitrypsin Deficiency | alpha1AT |
| Pompe Disease | hGAA |
| X-linked Retinoschisis | RS1 |
| Choroideremia | hCHM |
| Giant Axonal Neuropathy | JeT-GAN |
| X-linked Retinoschisis | hRS1 |
| Squamous Cell Head and Neck Cancer; | hAQP1 |
| Radiation Induced Xerostomia | |
| Hemophilia B | Factor IX |
| Homozygous FH | hLDLR |
| Dysferlinopathies | dysferlin transgene (e.g., |
| | rAAVrh74.MHCK7.DYSF.DV) |
| Hemophilia B | AAV6 ZFP nuclease |
| MPS I | AAV6 ZFP nuclease |
| Rheumatoid Arthritis | NF-kB.IFN-β |
| Batten/CLN6 | CLN6 |
| Sanfilippo Disease Type A | hSGSH |
| Osteoarthritis | 5IL-1Ra |
| Achromatopsia | CNGA3 |
| Achromatopsia | CNGB3 |
| Ornithine Transcarbamylase (OTC) | OTC |
| Deficiency | |
| Hemophilia A | Factor VIII |
| Mucopolysaccharidosis II | ZFP nuclease |
| Hemophilia A | ZFP nuclease |
| Wet AMD | anti-VEGF |
| X-Linked Retinitis Pigmentosa | RPGR |
| Mucopolysaccharidosis Type VI | hARSB |
| Leber Hereditary Optic Neuropathy | ND4 |
| X-Linked Myotubular Myopathy | MTM1 |
| Crigler-Najjar Syndrome | UGT1A1 |
| Achromatopsia | CNGB3 |
| Retinitis Pigmentosa | hPDE6B |
| X-Linked Retinitis Pigmentosa | RPGR |
| Mucopolysaccharidosis Type 3 B | hNAGLU |
| Duchenne Muscular Dystrophy | GALGT2 |
| Arthritis, Rheumatoid; Arthritis, | TNFR:Fc Fusion Gene |
| Psoriatic; Ankylosing Spondylitis | |
| Idiopathic Parkinson's Disease | Neurturin |
| Alzheimer's Disease | NGF |
| Human Immunodeficiency Virus | tgAAC09 |
| Infections; HIV Infections (HIV-1) | |
| Familial Lipoprotein Lipase Deficiency | LPL |
| Idiopathic Parkinson's Disease | Neurturin |
| Alpha-1 Antitrypsin Deficiency | hAAT |
| Leber Congenital Amaurosis (LCA) 2 | hRPE65v2 |
| Batten Disease; Late Infantile Neuronal | CLN2 |
| Lipofuscinosis | |
| Parkinson's Disease | GAD |
| Sanfilippo Disease Type | N-sulfoglucosamine sulfohydrolase |
| A/Mucopolysaccharidosis Type IIIA | (SGSH) gene |
| Congestive Heart Failure | SERC2a |
| Becker Muscular Dystrophy and Sporadic | Follistatin (e.g. rAAV.CMV.huFollistatin344) |
| Inclusion Body Myositis | |
| Parkinson's Disease | hAADC-2 |
| Choroideremia | REP1 |
| CEA Specific AAV-DC-CTL Treatment in | CEA |
| Stage IV Gastric Cancer | |
| Gastric Cancer | MUC1-peptide-DC-CTL |
| Leber's Hereditary Optical Neuropathy | scAAV2-P1ND4v2 |
| Aromatic Amino Acid Decarboxylase | hAADC |
| Deficiency | |
| Hemophilia B | Factor IX |
| Parkinson's Disease | AADC |
| Leber Hereditary Optic Neuropathy | Genetic: GS010\|Drug: Placebo |
| SMA - Spinal Muscular Atrophy\|Gene | SMN |
| Therapy | |
| Hemophilia A | B-Domain Deleted Factor VIII |
| MPS I | IDUA |
| MPS II | IDS |
| CLN3-Related Neuronal Ceroid- | CLN3 |
| Lipofuscinosis (Batten) | |
| Limb-Girdle Muscular Dystrophy, Type | hSGCB |
| 2E | |
| Alzheimer Disease | APOE2 |
| Retinitis Pigmentosa | hMERKTK |
| Retinitis Pigmentosa | RLBP1 |
| Wet AMD or diabetic retinopathy | Anti-VEGF antibody or Anti-VEGF trap (e.g., |
| | one or more extracellular domains of VEGFR-1 |
| | and/or VEGFR-2; e.g., aflibercept) |

TABLE 2B

| | | ANTIBODIES | |
|---|---|---|---|
| ANTIGENS | | (TRANSGENE) | INDICATIONS |
| Nervous | Amyloid beta | Solanezumab | Alzheimer's Disease |
| System | (Aβ or Abeta) | GSK933776 | |
| Targets | peptides | | |
| | derived | | |
| | from APP | | |
| | Sortilin | AL-001 | Frontotemporal dementia |
| | | | (FTD) |
| | Tau protein | ABBV-8E12 | Alzheimer's, Progressive |
| | | UCB-0107 | supranuclear palsy, |
| | | NI-105 (BIIB076) | frontotemporal |
| | | | demential, |
| | | | chronic traumatic |
| | | | encephalopathy, Pick's |
| | | | complex, primary age- |
| | | | related taupathy |
| | Semaphorin-4D | VX15/2503 | Huntington's disease, |
| | (SEMA4D) | | juvenile Huntington's |
| | | | disease |
| | alpha-synuclein | Prasinezumab | Parkinson's disease, |
| | | NI-202 (BIIB054) | synucleinopathies |
| | | MED-1341 | |
| | superoxide | NI-204 | ALS, Alzheimer's |
| | dismutase-1 | | Disease |
| | (SOD-1) | | |
| | CGRP Receptor | eptinezumab, | Migraines, Cluster |
| | | fremanezumab | headaches |
| | | galcanezumab | |
| Ocular Anti- | VEGF | Sevacizumab | diabetic |
| Angiogenic | | | retinopathy (DR), |
| Targets | | | myopic choroidal |
| | | | neovascularization |
| | | | (mCNV), age-related |
| | | | macular degeneration |
| | | | (AMD), macular edema |
| | VEGF | ranibizumab | Wet AMD |
| | | (LUCENTIS ®) | |
| | | bevacizumab | |
| | | (AVASTIN ®) | |
| | | brolucizumab | |
| | erythropoietin | LKA-651 | retinal vein occlusion |
| | receptor | | (RVO), wet AMD, |
| | | | macular edema |
| | Amyloid beta | Solanezumab | Dry AMD |
| | (Aβ or Abeta) | GSK933776 | |
| | peptides derived | | |
| | from APP | | |
| | activin receptor | ascrinvacumab | neovascular age-related |
| | like kinase 1 | | macular degeneration |
| | (ALK1) | | |
| | complement | tesidolumab | dry AMD, uveitis |
| | component 5 | | |
| | (C5) | | |
| | endoglin (END | carotuximab | wet AMD and other |
| | or CD105) | | retinal disorders |
| | | | caused by increased |
| | | | vascularization |
| | complement | ANX-007 | glaucoma |
| | component 1Q | | |
| | (C1Q) | | |
| TNF-alpha | | adalimumab | uveitis |
| | | (HUMIRA ®) | |
| | | infliximab | |
| | | (REMICADE ®) | |
| | | golimumab | |
| Repulsive guidance molecule-A | | elezanumab | multiple sclerosis |
| Transthyretin (TTR) | | NI-301 | amyloidosis |
| | | PRX-004 | |
| Connective tissue growth factor | | pamrevlumab | fibrotic diseases, e.g., |
| (CTGF) | | | diabetic nephropathy, liver |
| | | | fibrosis, idiopathic |
| | | | pulmonary fibrosis |
| Neuromyelitis | interleukin | Satralizumab | NMO, DR, DME, uveitis |
| optica | receptor 6 | sarilumab | |
| (NMO)/Uveitis | (IL6R) | | |
| targets | CD19 | inebilizumab | NMO |

| Integrin beta 7 | etrolizumab | ulcerative colitis, Crohn's |
|---|---|---|
| | | disease |
| Sclerostin | romosozumab | Osteoporosis, abnormal |
| | (EVENITY ®) | bone loss or weakness |

TABLE 2C

| | | ANTIBODIES | |
|---|---|---|---|
| ANTIGENS | | (TRANSGENE) | INDICATIONS |
| | Amyloid beta (Aβ | Aducanumab | Alzheimer's Disease |
| | or Abeta) peptides | crenezumab | |
| | | gantenerumab | |
| Nervous System | Tau protein | anti-TAU | Alzheimer's, Progressive |
| Targets | | | supranuclear palsy, |
| | | | frontotemporal demential, |
| | | | chronic traumatic |
| | | | encephalopathy, Pick's |
| | | | complex, primary age- |
| | | | related taupathy |
| | CGRP Receptor | erenumab | Migraine |
| | | (AIMOVIG ™) | |
| Interleukins or | IL-17A | ixekizumab | Plaque psoriasis, psoriatic |
| interleukin | | (TALTZ ®) | arthritis, ankylosing |
| receptors | | secukinumab | sponylitis |
| | | (COSENTYX ®) | |
| | IL-5 | mepolizumab | Asthma |
| | | (NUCALA ®) | |
| | IL-12/IL-23 | ustekinumab | Psoriasis & Crohn's disease |
| | | (STELARA ®) | |
| | IL-4R | dupilumab | Atopic dermatitis |
| Integrin | | vedolizumab | Ulcerative colitis & |
| | | (ENTYVIO ®) | Crohn's disease |
| | | Natalizumab (anti- | Multiple sclerosis & |
| | | integrin alpha 4) | Crohn's disease |
| Cardiovascular | PCSK9 | alirocumab | HeFH & HoFH |
| Targets | | (PRALUENT ®) | |
| | | evolucomab | |
| | | (REPATHA ®) | |
| | ANGPTL3 | evinacumab | HoFH & severe forms of |
| | | | dyslipidema |
| | Proinflammatory/ | E06-scFv | Cardiovascular diseases |
| | proatherogenic | | such as atherosclerosis |
| | phospholipids | | |
| RANKL | | denosumab | Osteoporosis, increasing |
| | | (XGEVA ® and | bone mass in breast and |
| | | PROLIA ®) | prostate cancer patients, & |
| | | | preventing skeletal-related |
| | | | events due to bone |
| | | | metastasis |
| PD-1, or PD-L1 or PD-L2 | | nivolumab | Metastatic melanoma, |
| | | (OPDIVO ®) | lymphoma, non-small cell |
| | | pembrolizumab | lung carcinoma |
| | | (KEYTRUDA ®) | |
| BLyS (B-lymphocyte stimulator, also | | belimumab | Systemic lupus |
| known as B-cell activating factor (BAFF)) | | (BENLYSTA ®) | erythromatosis |
| Ocular Targets | Factor D | lampalizumab | Dry AMD |
| | MMP9 | andecaliximab | Dry AMD |
| TNF-alpha | | adalimumab | Rheumatoid arthritis, |
| | | (HUMIRA ®) and | psoriatic arthritis, |
| | | infliximab | askylosing spondylitis, |
| | | (REMICADE ® | Crohn's disease, plaque |
| | | | psoriasis, ulcerative colitis |
| Plasma Protein | C5, C5a | cculizumab | Paroxysmal nocturnal |
| targets | | (SOLIRIS ®) | hemoglobinuria, atypical |
| | | | hemolytic uremic |
| | | | syndrome, complement- |
| | | | mediated thrombotic |
| | | | microangiopathy |
| | Plasma kallikrein | lanadelumab | Hereditary angioedema |
| | | | (HAE) |

In some embodiments, the rAAV particles are rAAV viral vectors encoding an anti-VEGF Fab. In specific embodiments, the rAAV particles are rAAV8-based viral vectors encoding an anti-VEGF Fab. In more specific embodiments, the rAAV particles are rAAV8-based viral vectors encoding ranibizumab. In some embodiments, the rAAV particles are rAAV viral vectors encoding iduronidase (IDUA). In specific embodiments, the rAAV particles are rAAV9-based viral vectors encoding IDUA. In some embodiments, the rAAV particles are rAAV viral vectors encoding iduronate 2-sulfatase (IDS). In specific embodiments, the rAAV particles are rAAV9-based viral vectors encoding IDS. In some embodiments, the rAAV particles are rAAV viral vectors encoding a low-density lipoprotein receptor (LDLR). In specific embodiments, the rAAV particles are rAAV8-based viral vectors encoding LDLR. In some embodiments, the rAAV particles are rAAV viral vectors encoding tripeptidyl peptidase 1 (TPP1) protein. In specific embodiments, the rAAV particles are rAAV9-based viral vectors encoding TPP1. In some embodiments, the rAAV particles are rAAV viral vectors encoding non-membrane associated splice variant of VEGF receptor 1 (sFlt-1). In some embodiments, the rAAV particles are rAAV viral vectors encoding gamma-sarcoglycan, Rab Escort Protein 1 (REP1/CHM), retinoid isomerohydrolase (RPE65), cyclic nucleotide gated channel alpha 3 (CNGA3), cyclic nucleotide gated channel beta 3 (CNGB3), aromatic L-amino acid decarboxylase (AADC), lysosome-associated membrane protein 2 isoform B (LAMP2B), Factor VIII, Factor IX, retinitis pigmentosa GTPase regulator (RPGR), retinoschisin (RS1), sarcoplasmic reticulum calcium ATPase (SERCA2a), aflibercept, battenin (CLN3), transmembrane ER protein (CLN6), glutamic acid decarboxylase (GAD), Glial cell line-derived neurotrophic factor (GDNF), aquaporin 1 (AQP1), dystrophin, microdystrophin, myotubularin 1 (MTM1), follistatin (FST), glucose-6-phosphatase (G6Pase), apolipoprotein A2 (APOA2), uridine diphosphate glucuronosyl transferase 1A1 (UGT1A1), arylsulfatase B (ARSB), N-acetyl-alpha-glucosaminidasc (NAGLU), alpha-glucosidase (GAA), alpha-galactosidase (GLA), beta-galactosidase (GLB1), lipoprotein lipase (LPL), alpha 1-antitrypsin (AAT), phosphodiesterase 6B (PDE6B), ornithine carbamoyltransferase 90TC), survival motor neuron (SMN1), survival motor neuron (SMN2), neurturin (NRTN), Neurotrophin-3 (NT-3/NTF3), porphobilinogen deaminase (PBGD), nerve growth factor (NGF), mitochondrially encoded NADH: ubiquinone oxidoreductase core subunit 4 (MT-ND4), protective protein cathepsin A (PPCA), dysferlin, MER proto-oncogene, tyrosine kinase (MERTK), cystic fibrosis transmembrane conductance regulator (CFTR), or tumor necrosis factor receptor (TNFR)-immunoglobulin (IgG1) Fc fusion.

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, rAAV particles comprise a capsid containing a capsid protein chimeric of two or more AAV capsid serotypes. In some embodiments, the capsid protein is a chimeric of 2 or more AAV capsid proteins from AAV serotypes selected from AAV1, AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In certain embodiments, a single-stranded AAV (ssAAV) can be used. In certain embodiments, a self-complementary vector, e.g., scAAV, can be used (see, e.g., Wu, 2007, Human Gene Therapy, 18 (2): 171-82, McCarty et al, 2001, Gene Therapy, Vol. 8, Number 16, Pages 1248-1254; and U.S. Pat. Nos. 6,596,535; 7,125,717; and 7,456,683, each of which is incorporated herein by reference in its entirety).

In some embodiments, the rAAV particles comprise a capsid protein from an AAV capsid serotype selected from AAV8 or AAV9. In some embodiments, the rAAV particles have an AAV capsid serotype of AAV8. In some embodiments, the rAAV particles have an AAV capsid scrotype of AAV9.

In some embodiments, the rAAV particles comprise a capsid protein that is a derivative, modification, or pseudotype of AAV8 or AAV9 capsid protein. In some embodiments, the rAAV particles comprise a capsid protein that has an AAV8 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV8 capsid protein.

In some embodiments, the rAAV particles comprise a capsid protein that is a derivative, modification, or pseudotype of AAV9 capsid protein. In some embodiments, the rAAV particles comprise a capsid protein that has an AAV9 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV9 capsid protein.

In additional embodiments, the rAAV particles comprise a mosaic capsid. Mosaic AAV particles are composed of a mixture of viral capsid proteins from different serotypes of AAV. In some embodiments, the rAAV particles comprise a mosaic capsid containing capsid proteins of a serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise a mosaic capsid containing capsid proteins of a serotype selected from AAV1, AAV2, AAV5, AAV6, AAV7, AAV8, AAV9, AAV10, AAVrh.8, AAVrh.10, AAVhu.37, AAVrh.20, and AAVrh.74.

In additional embodiments, the rAAV particles comprise a pseudotyped rAAV particle. In some embodiments, the pseudotyped rAAV particle comprises (a) a nucleic acid vector comprising AAV ITRs and (b) a capsid comprised of capsid proteins derived from AAVx (e.g., AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16). In additional embodiments, the rAAV particles comprise a pseudotyped rAAV particle comprised of a capsid protein of an AAV serotype selected from AAV1, AAV2, AAV5, AAV6, AAV7, AAV8, AAV9, AAV10, AAVrh.8, and AAVrh. 10, AAVhu.37, AAVrh.20, and AAVrh.74. In additional embodiments, the rAAV particles comprise a pseudotyped rAAV particle containing AAV8 capsid protein. In additional embodiments, the rAAV particles comprise a pseudotyped rAAV particle comprised of AAV9 capsid protein. In some embodiments, the pseudotyped rAAV8 or rAAV9 particles are rAAV2/8 or rAAV2/9 pseudotyped particles. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, the rAAV particles comprise a capsid containing a capsid protein chimeric of two or more AAV capsid serotypes. In some embodiments, the rAAV particles comprise an AAV capsid protein chimeric of AAV8 capsid protein and one or more AAV capsid proteins from an AAV serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise an AAV capsid protein chimeric of AAV8 capsid protein and one or more AAV capsid proteins from an AAV serotype selected from AAV1, AAV2, AAV5, AAV6, AAV7, AAV9, AAV10, rAAVrh10, AAVrh.8, AAVrh.10, AAVhu.37, AAVrh.20, and AAVrh.74. In some embodiments, the rAAV particles comprise an AAV capsid protein chimeric of AAV9 capsid protein the capsid protein of one or more AAV capsid serotypes selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise an AAV capsid protein chimeric of AAV9 capsid protein the capsid protein of one or more AAV capsid serotypes selected from AAV1, AAV2, AAV3, AAV4, AAV5, AA6, AAV7, AAV8, AAV9, AAVrh.8, AAVrh.10, AAVhu.37, AAVrh.20, and AAVrh.74.

### Methods for Isolating RAAV Particles

In some embodiments, the disclosure provides methods for producing recombinant adeno-associated virus (rAAV) particles, comprising isolating rAAV particles from a feed comprising an impurity (for example, rAAV production culture). In some embodiments, a method for producing recombinant adeno-associated virus (rAAV) particles described herein comprises (a) isolating rAAV particles from a feed comprising an impurity (for example, rAAV production culture), and (b) formulating the isolated rAAV particles to produce the formulation.

In some embodiments, the disclosure further provides methods for producing a pharmaceutical unit dosage of a formulation comprising isolated recombinant adeno-associated virus (rAAV) particles, comprising isolating rAAV particles from a feed comprising an impurity (for example, rAAV production culture), and formulating the isolated rAAV particles.

Isolated rAAV particles can be isolated using methods known in the art. In some embodiments, methods of isolating rAAV particles comprises downstream processing such as, for example, harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, hydroxylapatite chromatography, sterile filtration, or any combination(s) thereof. In some embodiments, downstream processing includes at least 2, at least 3, at least 4, at least 5 or at least 6 of: harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, hydroxylapatite chromatography, and sterile filtration. In some embodiments, downstream processing comprises harvest of a cell culture, clarification of the harvested cell culture (e.g., by depth filtration), sterile filtration, tangential flow filtration, affinity chromatography, and anion exchange chromatography. In some embodiments, downstream processing comprises clarification of a harvested cell culture, sterile filtration, tangential flow filtration, affinity chromatography, and anion exchange chromatography. In some embodiments, downstream processing comprises clarification of a harvested cell culture by depth filtration, sterile filtration, tangential flow filtration, affinity chromatography, and anion exchange chromatography. In some embodiments, clarification of the harvested cell culture comprises sterile filtration. In some embodiments, downstream processing does not include centrifugation. In some embodiments, the rAAV particles comprise a capsid protein of the AAV8 serotype. In some embodiments, the rAAV particles comprise a capsid protein of the AAV9 serotype.

In some embodiments, a method of isolating rAAV particles produced according to a method described herein comprises harvest of a cell culture, clarification of the harvested cell culture (e.g., by depth filtration), a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles described herein comprises harvest of a cell culture, clarification of the harvested cell culture (e.g., by depth filtration), a first sterile filtration, affinity chromatography, anion exchange chromatography (e.g., monolith anion exchange chromatography or AEX chromatography using a quaternary amine ligand), a tangential flow filtration, and a second sterile filtration. In some embodiments, a method of isolating rAAV particles produced according to a method described herein comprises clarification of a harvested cell culture, a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles described herein comprises clarification of a harvested cell culture, a first sterile filtration, affinity chromatography, anion exchange chromatography (e.g., monolith anion exchange chromatography or AEX chromatography using a quaternary amine ligand), tangential flow filtration, and a second sterile filtration. In some embodiments, a method of isolating rAAV particles produced according to a method described herein comprises clarification of a harvested cell culture by depth filtration, a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles described herein comprises clarification of a harvested cell culture by depth filtration, a first sterile filtration, affinity chromatography, anion exchange chromatography (e.g., monolith anion exchange chromatography or AEX chromatography using a quaternary amine ligand), tangential flow filtration, and a second sterile filtration. In some embodiments, the method does not include centrifugation. In some embodiments, clarification of the harvested cell culture comprises sterile filtration. In some embodiments, the rAAV particles comprise a capsid protein of the AAV8 serotype. In some embodiments, the rAAV particles comprise a capsid protein of the AAV9 serotype.

Numerous methods are known in the art for production of rAAV particles, including transfection, stable cell line production, and infectious hybrid virus production systems which include adenovirus-AAV hybrids, herpesvirus-AAV hybrids and baculovirus-AAV hybrids. rAAV production cultures for the production of rAAV virus particles all require; (1) suitable host cells, including, for example, human-derived cell lines such as HeLa, A549, or HEK293 cells and their derivatives (HEK293T cells, HEK293F cells), mammalian cell lines such as Vero, or insect-derived cell lines such as SF-9 in the case of baculovirus production systems; (2) suitable helper virus function, provided by wild type or mutant adenovirus (such as temperature sensitive adenovirus), herpes virus, baculovirus, or a plasmid construct providing helper functions; (3) AAV rep and cap genes and gene products; (4) a transgene (such as a therapeutic transgene) flanked by AAV ITR sequences; and (5) suitable media and media components to support rAAV production. In some embodiments, the suitable helper virus function is provided by a recombinant polynucleotide described herein or a plasmid described herein. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), and Sf-900 II SFM media as described in U.S. Pat. No. 6,723,551, which is incorporated herein by reference in its entirety.

rAAV production cultures can routinely be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, RK cells, Per.C6 cells, chicken embryo cells or SF-9 cells which can be cultured in a variety of ways including, for example, spinner flasks, stirred tank bioreactors, and disposable systems such as the Wave bag system. In some embodiments, the cells are HEK293 cells. In some embodiments, the cells are HEK293 cells adapted for growth in suspension culture. Numerous suspension cultures are known in the art for production of rAAV particles, including for example, the cultures disclosed in U.S. Pat. Nos. 6,995,006, 9,783,826, and in U.S. Pat. Appl. Pub. No. 20120122155, each of which is incorporated herein by reference in its entirety.

In some embodiments, the rAAV production culture comprises a high density cell culture. In some embodiments, the culture has a total cell density of between about 1×10E+06 cells/ml and about 30×10E+06 cells/ml. In some embodiments, more than about 50% of the cells are viable cells. In some embodiments, the cells are HeLa cells, HEK293 cells, HEK293 derived cells (e.g., HEK293T cells, HEK293F cells), Vero cells, or SF-9 cells. In further embodiments, the cells are HEK293 cells. In further embodiments, the cells are HEK293 cells adapted for growth in suspension culture.

In additional embodiments of the provided method the rAAV production culture comprises a suspension culture comprising rAAV particles. Numerous suspension cultures are known in the art for production of rAAV particles, including for example, the cultures disclosed in U.S. Pat. Nos. 6,995,006, 9,783,826, and in U.S. Pat. Appl. Pub. No. 20120122155, each of which is incorporated herein by reference in its entirety. In some embodiments, the suspension culture comprises a culture of mammalian cells or insect cells. In some embodiments, the suspension culture comprises a culture of HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, RK cells, Per.C6 cells, chicken embryo cells or SF-9 cells. In some embodiments, the suspension culture comprises a culture of HEK293 cells.

In some embodiments, methods for the production of rAAV particles encompasses providing a cell culture comprising a cell capable of producing rAAV; adding to the cell culture a histone deacetylase (HDAC) inhibitor to a final concentration between about 0.1 mM and about 20 mM; and maintaining the cell culture under conditions that allows production of the rAAV particles. In some embodiments, the HDAC inhibitor comprises a short-chain fatty acid or salt thereof. In some embodiments, the HDAC inhibitor comprises butyrate (e.g., sodium butyrate), valproate (e.g., sodium valproate), propionate (e.g., sodium propionate), or a combination thereof.

In some embodiments, rAAV particles are produced as disclosed in WO 2020/033842, which is incorporated herein by reference in its entirety.

Recombinant AAV particles can be harvested from rAAV production cultures by harvest of the production culture comprising host cells or by harvest of the spent media from the production culture, provided the cells are cultured under conditions known in the art to cause release of rAAV particles into the media from intact host cells. Recombinant AAV particles can also be harvested from rAAV production cultures by lysis of the host cells of the production culture. Suitable methods of lysing cells are also known in the art and include for example multiple freeze/thaw cycles, sonication, microfluidization, and treatment with chemicals, such as detergents and/or proteases.

At harvest, rAAV production cultures can contain one or more of the following: (1) host cell proteins; (2) host cell DNA; (3) plasmid DNA; (4) helper virus; (5) helper virus proteins; (6) helper virus DNA; and (7) media components including, for example, serum proteins, amino acids, transferrins and other low molecular weight proteins. rAAV production cultures can further contain product-related impurities, for example, inactive vector forms, empty viral capsids, aggregated viral particles or capsids, mis-folded viral capsids, degraded viral particle.

In some embodiments, the rAAV production culture harvest is clarified to remove host cell debris. In some embodiments, the production culture harvest is clarified by filtration through a series of depth filters. Clarification can also be achieved by a variety of other standard techniques known in the art, such as, centrifugation or filtration through any cellulose acetate filter of 0.2 mm or greater pore size known in the art. In some embodiments, clarification of the harvested cell culture comprises sterile filtration. In some embodiments, the production culture harvest is clarified by centrifugation. In some embodiments, clarification of the production culture harvest does not include centrifugation.

In some embodiments, harvested cell culture is clarified using filtration. In some embodiments, clarification of the harvested cell culture comprises depth filtration. In some embodiments, clarification of the harvested cell culture further comprises depth filtration and sterile filtration. In some embodiments, harvested cell culture is clarified using a filter train comprising one or more different filtration media. In some embodiments, the filter train comprises a depth filtration media. In some embodiments, the filter train comprises one or more depth filtration media. In some embodiments, the filter train comprises two depth filtration media. In some embodiments, the filter train comprises a sterile filtration media. In some embodiments, the filter train comprises 2 depth filtration media and a sterile filtration media. In some embodiments, the depth filter media is a porous depth filter. In some embodiments, the filter train comprises Clarisolve® 20 MS, Millistak+® COHC, and a sterilizing grade filter media. In some embodiments, the filter train comprises Clarisolve® 20 MS, Millistak+® COHC, and Sartopore® 2 XLG 0.2 μm. In some embodiments, the harvested cell culture is pretreated before contacting it with the depth filter. In some embodiments, the pretreating comprises adding a salt to the harvested cell culture. In some embodiments, the pretreating comprises adding a chemical flocculent to the harvested cell culture. In some embodiments, the harvested cell culture is not pre-treated before contacting it with the depth filter.

In some embodiments, the production culture harvest is clarified by filtration are disclosed in WO 2019/212921, which is incorporated herein by reference in its entirety.

In some embodiments, the rAAV production culture harvest is treated with a nuclease (e.g., Benzonase®) or endonuclease (e.g., endonuclease from Serratia marcescens) to digest high molecular weight DNA present in the production culture. The nuclease or endonuclease digestion can routinely be performed under standard conditions known in the art. For example, nuclease digestion is performed at a final concentration of 1-2.5 units/ml of Benzonase® at a temperature ranging from ambient to 37° C. for a period of 30 minutes to several hours.

Sterile filtration encompasses filtration using a sterilizing grade filter media. In some embodiments, the sterilizing grade filter media is a 0.2 or 0.22 μm pore filter. In some embodiments, the sterilizing grade filter media comprises polyethersulfone (PES). In some embodiments, the sterilizing grade filter media comprises polyvinylidene fluoride (PVDF). In some embodiments, the sterilizing grade filter media has a hydrophilic heterogeneous double layer design. In some embodiments, the sterilizing grade filter media has a hydrophilic heterogeneous double layer design of a 0.8 μm pre-filter and 0.2 μm final filter membrane. In some embodiments, the sterilizing grade filter media has a hydrophilic heterogeneous double layer design of a 1.2 μm pre-filter and 0.2 μm final filter membrane. In some embodiments, the sterilizing grade filter media is a 0.2 or 0.22 μm pore filter. In further embodiments, the sterilizing grade filter media is a 0.2 μm pore filter. In some embodiments, the sterilizing grade filter media is a Sartopore® 2 XLG 0.2 μm, Durapore™ PVDF Membranes 0.45 μm, or Sartoguard® PES 1.2 μm+0.2 μm nominal pore size combination. In some embodiments, the sterilizing grade filter media is a Sartopore® 2 XLG 0.2 μm.

In some embodiments, the clarified feed is concentrated via tangential flow filtration (“TFF”) before being applied to a chromatographic medium, for example, affinity chromatography medium. Large scale concentration of viruses using TFF ultrafiltration has been described by Paul et al., Human Gene Therapy 4:609-615 (1993). TFF concentration of the clarified feed enables a technically manageable volume of clarified feed to be subjected to chromatography and allows for more reasonable sizing of columns without the need for lengthy recirculation times. In some embodiments, the clarified feed is concentrated between at least two-fold and at least ten-fold. In some embodiments, the clarified feed is concentrated between at least ten-fold and at least twenty-fold. In some embodiments, the clarified feed is concentrated between at least twenty-fold and at least fifty-fold. In some embodiments, the clarified feed is concentrated about twenty-fold. One of ordinary skill in the art will also recognize that TFF can also be used to remove small molecule impurities (e.g., cell culture contaminants comprising media components, serum albumin, or other serum proteins) form the clarified feed via diafiltration. In some embodiments, the clarified feed is subjected to diafiltration to remove small molecule impurities. In some embodiments, the diafiltration comprises the use of between about 3 and about 10 diafiltration volume of buffer. In some embodiments, the diafiltration comprises the use of about 5 diafiltration volume of buffer. One of ordinary skill in the art will also recognize that TFF can also be used at any step in the purification process where it is desirable to exchange buffers before performing the next step in the purification process. In some embodiments, the methods for isolating rAAV from the clarified feed described herein comprise the use of TFF to exchange buffers.

Affinity chromatography can be used to isolate rAAV particles from a composition. In some embodiments, affinity chromatography is used to isolate rAAV particles from the clarified feed. In some embodiments, affinity chromatography is used to isolate rAAV particles from the clarified feed that has been subjected to tangential flow filtration. Suitable affinity chromatography media are known in the art and include without limitation, AVB Sepharose™, POROS™ CaptureSelect™ AAVX affinity resin, POROS™ CaptureSelect™ AAV9 affinity resin, and POROS™ CaptureSelect™ AAV8 affinity resin. In some embodiments, the affinity chromatography media is POROS™ CaptureSelect™ AAV9 affinity resin. In some embodiments, the affinity chromatography media is POROS™ CaptureSelect™ AAV8 affinity resin. In some embodiments, the affinity chromatography media is POROS™ CaptureSelect™ AAVX affinity resin.

Anion exchange chromatography can be used to isolate rAAV particles from a composition. In some embodiments, anion exchange chromatography is used after affinity chromatography as a final concentration and polish step. Suitable anion exchange chromatography media are known in the art and include without limitation, UNOsphere™ Q (Biorad, Hercules, Calif.), and N-charged amino or imino resins such as e.g., POROS™ 50 PI, or any DEAE, TMAE, tertiary or quaternary amine, or PEI-based resins known in the art (U.S. Pat. No. 6,989,264; Brument et al., Mol. Therapy 6 (5): 678-686 (2002); Gao et al., Hum. Gene Therapy 11:2079-2091 (2000)). In some embodiments, the anion exchange chromatography media comprises a quaternary amine. In some embodiments, the anion exchange media is a monolith anion exchange chromatography resin. In some embodiments, the monolith anion exchange chromatography media comprises glycidylmethacrylate-ethylenedimethacrylate or styrene-divinylbenzene polymers. In some embodiments, the monolith anion exchange chromatography media is selected from the group consisting of CIMmultus™ QA-1 Advanced Composite Column (Quaternary amine), CIMmultus™ DEAE-1 Advanced Composite Column (Dicthylamino), CIM® QA Disk (Quaternary amine), CIM® DEAE, and CIM® EDA Disk (Ethylene diamino). In some embodiments, the monolith anion exchange chromatography media is CIMmultus™ QA-1 Advanced Composite Column (Quaternary amine). In some embodiments, the monolith anion exchange chromatography media is CIM® QA Disk (Quaternary amine). In some embodiments, the anion exchange chromatography media is CIM QA (BIA Separations, Slovenia). In some embodiments, the anion exchange chromatography media is BIA CIM® QA-80 (Column volume is 80 mL). One of ordinary skill in the art can appreciate that wash buffers of suitable ionic strength can be identified such that the rAAV remains bound to the resin while impurities, including without limitation impurities which may be introduced by upstream purification steps are stripped away.

In some embodiments, anion exchange chromatography is performed according to a method disclosed in WO 2019/241535, which is incorporated herein by reference in its entirety.

In some embodiments, a method of isolating rAAV particles comprises determining the vector genome titer, capsid titer, and/or the ratio of full to empty capsids in a composition comprising the isolated rAAV particles. In some embodiments, the vector genome titer is determined by quantitative PCR (qPCR) or digital PCR (dPCR) or droplet digital PCR (ddPCR). In some embodiments, the capsid titer is determined by serotype-specific ELISA. In some embodiments, the ratio of full to empty capsids is determined by Analytical Ultracentrifugation (AUC) or Transmission Electron Microscopy (TEM).

In some embodiments, the vector genome titer, capsid titer, and/or the ratio of full to empty capsids is determined by spectrophotometry, for example, by measuring the absorbance of the composition at 260 nm; and measuring the absorbance of the composition at 280 nm. In some embodiments, the rAAV particles are not denatured prior to measuring the absorbance of the composition. In some embodiments, the rAAV particles are denatured prior to measuring the absorbance of the composition. In some embodiments, the absorbance of the composition at 260 nm and 280 nm is determined using a spectrophotometer. In some embodiments, the absorbance of the composition at 260 nm and 280 nm is determined using an HPLC. In some embodiments, the absorbance is peak absorbance. Several methods for measuring the absorbance of a composition at 260 nm and 280 nm are known in the art. Methods of determining vector genome titer and capsid titer of a composition comprising the isolated recombinant rAAV particles are disclosed in WO 2019/212922, which is incorporated herein by reference in its entirety.

In additional embodiments the disclosure provides compositions comprising isolated rAAV particles produced according to a method described herein. In some embodiment, the composition is a pharmaceutical composition comprising a pharmaceutically acceptable carrier.

As used herein the term “pharmaceutically acceptable” means a biologically acceptable formulation, gaseous, liquid or solid, or mixture thereof, which is suitable for one or more routes of administration, in vivo delivery or contact. A “pharmaceutically acceptable” composition is a material that is not biologically or otherwise undesirable, e.g., the material may be administered to a subject without causing substantial undesirable biological effects. Thus, such a pharmaceutical composition may be used, for example in administering rAAV isolated according to the disclosed methods to a subject. Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions. Pharmaceutical compositions can be formulated to be compatible with a particular route of administration or delivery, as set forth herein or known to one of skill in the art. Thus, pharmaceutical compositions include carriers, diluents, or excipients suitable for administration by various routes. Pharmaceutical compositions and delivery systems appropriate for rAAV particles and methods and uses of the invention are known in the art (see, e.g., Remington: The Science and Practice of Pharmacy (2003) 20th ed., Mack Publishing Co., Easton, Pa.; Remington's Pharmaceutical Sciences (1990) 18th ed., Mack Publishing Co., Easton, Pa.; The Merck Index (1996) 12th cd., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11th ed., Lippincott Williams & Wilkins, Baltimore, Md.; and Poznansky et al., Drug Delivery Systems (1980), R. L. Juliano, ed., Oxford, N.Y., pp. 253-315).

In some embodiments, the composition is a pharmaceutical unit dose. A “unit dose” refers to a physically discrete unit suited as a unitary dosage for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dose forms may be within, for example, ampules and vials, which may include a liquid composition, or a composition in a freeze-dried or lyophilized state; a sterile liquid carrier, for example, can be added prior to administration or delivery in vivo. Individual unit dose forms can be included in multi-dose kits or containers. Recombinant vector (e.g., AAV) sequences, plasmids, vector genomes, and recombinant virus particles, and pharmaceutical compositions thereof can be packaged in single or multiple unit dose form for ease of administration and uniformity of dosage. In some embodiments, the composition comprises rAAV particles comprising an AAV capsid protein from an AAV capsid serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the AAV capsid serotype is AAV8. In some embodiments, the AAV capsid serotype is AAV9.

### EXAMPLES

### Example 1. Development of Improved Helper Plasmids

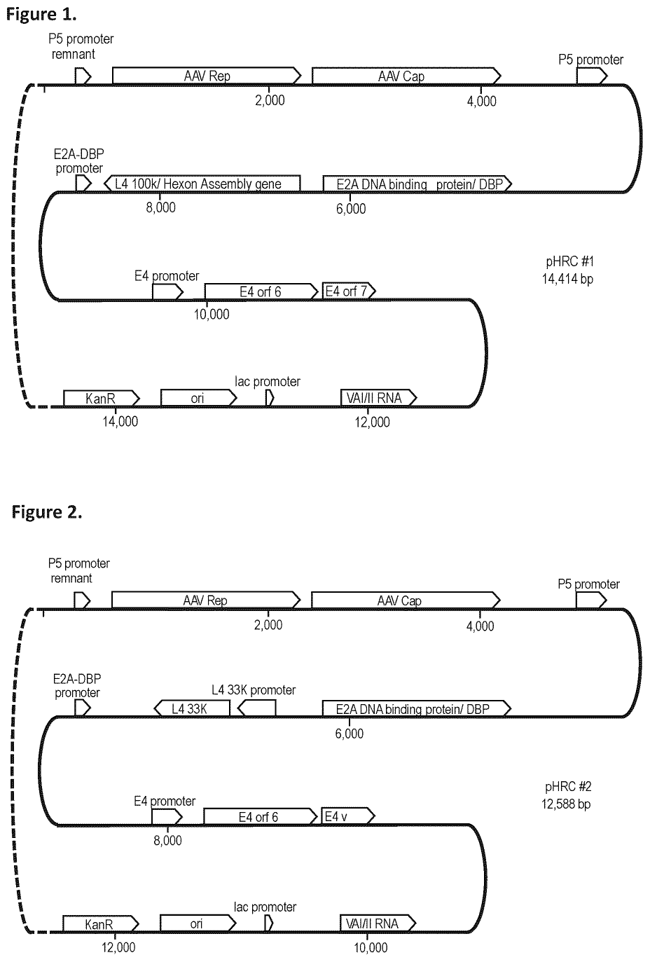

Plasmid pAdDeltaF6, also referred to as the original helper plasmid, was constructed by Dr. James M. Wilson and colleagues at UPenn. pAdDeltaF6 is 15770 bp in size. The plasmid contains the regions of the adenovirus genome that are important for AAV replication, namely E2A (DNA binding protein), E4, and VA RNAI but does not contain other adenovirus replication genes. This plasmid was derived from an E1, E3 deleted molecular clone of Ad5 (pBHG10, a pBR322 based plasmid). Deletions were introduced in the Ad5 DNA to remove expression of unnecessary adenovirus genes and reduce the amount of adenovirus DNA from 32 kb to 12 kb (FIG. 1, A of International Publication No. WO2023060113A, which is incorporated herein by reference in its entirety). Finally, the ampicillin resistance gene was replaced by the kanamycin resistance gene to give pAdDeltaF6 (FIG. 1, B of International Publication No. WO2023060113A). The functional elements of the E2A, E4 and VA RNAI adenoviral genes necessary for AAV vector production remain in this plasmid. The adenoviral E1 essential gene functions are supplied by the HEK293 cells. There are also some remnant genes/elements that were resulted from partial digestion of pBHG10. These include the promoterless L3 23K/viral endoprotease, L4 100K/hexon assembly gene, L4 pVIII/hexon-associated precursor and L5 pVI/fiber genes in the map. In pAdDeltaF6 plasmid, these genes are not transcribed due to the deletion of their promoter MLP (Major Late Promoter). Biasiotto et al., Int. J. Mol. Sci., 16:2893-2912; doi: 10.3390/ijms16022893 (2015). Since some of these genes including L4 100K and L4 pVIII overlap with E2A region, deletion of these genes may impact the production of the essential helper protein E2A as described below during the sequential reconfiguration of the helper plasmid. Furthermore, there is an L4 22K/33K gene with its own intact promoter located at this region. This gene encodes the L4 22K and L4 33K proteins involved in Adenovirus 5 packaging. The promoter of the L4 22K/33K gene also overlaps with E2A region. Therefore, deletion of the promoter may impact the production of E2A. There is a partial adenoviral inverted terminal repeat in the plasmid map that also resulted from partial digestion of pBHG10. However, due to the deletion of the essential DNA polymerase gene (E2 region) for Adenovirus 5 DNA replication, no infectious adenovirus is expected to be generated. DNA plasmid sequencing was performed by Qiagen Genomic Services and revealed 100% homology with the following important functional elements of the reference sequence pAdDeltaF6 p1707FH-Q: E4 ORF6 3692-2808 bp; E2A DNA binding protein 11784-10194 bp; VA RNAI region 12426-13378 bp. The sequence is confirmed at Aldevron, as part of the manufacturing process.

New helper plasmid #1 The new helper plasmid #1 (FIG. 2 of International Publication No. WO2023060113A) was constructed based on Ad5 sequence where E2A and E4 orientations were re-configured to express them bidirectionally. The rationale behind this was to avoid possible interference from E4 strong promoter which could result in lowering the expression from E2A promoter located downstream. The new helper plasmid #1 genes were synthesized by Genscript and cloned into EcoRI/NotI sites of pUC57 vector that was freely available from Genscript. In this new designed plasmid, some nonessential remnant genes (Ad5 structural genes) and elements that include the ITR sequence (Ad5 inverted terminal repeat) next to E4 promoter, L3 23K/viral endoprotease, L5 pVI/fibre, and L4 pVIII/hexon-associated precursor sequences were removed. On the other hand, the L4 33K/L4 100K hexon assembly gene was kept since the E2A transcription starting sites (TSS) are located at that region and their removal may impact E2A expression. The virus associated (VA) RNA was further modified by incorporating VA RNAII to VA RNAI. VA RNA is known to stimulate viral protein synthesis in infected cells and antagonizes the interferon-induced cellular defense system by regulating innate cellular response (Ma et al., Journal of Virology, August 1996, p 5083-5099). The new plasmid has the size of 11,484 bp.

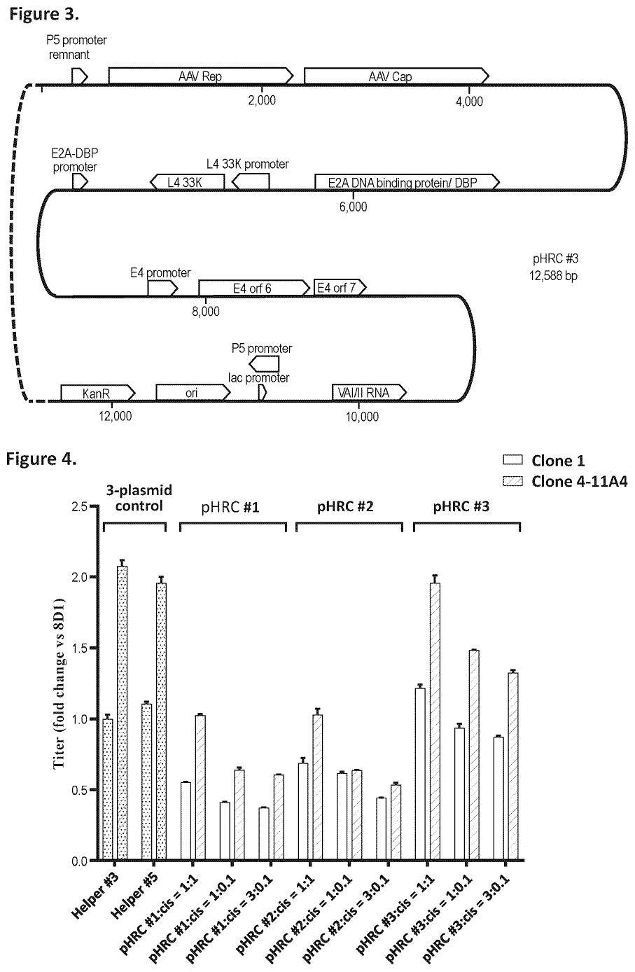

The new helper plasmid #1 improved AAV titers and performed well on different transgenes as shown in FIG. 3 of International Publication No. WO2023060113A. rAAV production titers were assessed using clone 1, 2, 3, 4, and 5 HEK293-derived host cells.

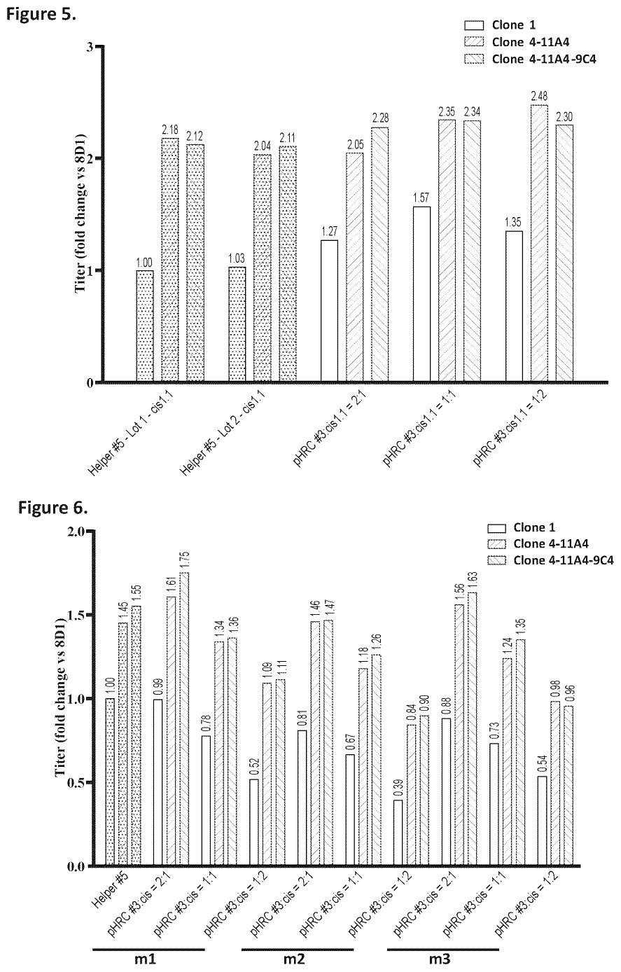

New helper plasmid #2 The new helper plasmid #2 (FIG. 4 of International Publication No. WO2023060113A) was designed based on the new helper #1. In this new design, the E4 region was dissected by sequential deletion and the impact of the deletions on AAV production was investigated. E4 Orf 1 and 2 were deleted based on results indicating that deletion of E4 Orf 1 and 2 improved AAV titers (data not shown). It is known in the field that the promoter controlling E4 region is active at earlier phase of adenovirus infection and continues to the late phases. The E4 region has the potential to transcribe and encode 7 different proteins that are resulted from differential splicing of a single primary transcript (Orf1, 2, 3, 3/4, 4, 6, 6/7) generated by this promoter. The pattern of differential splicing for this transcript changes during the phases of viral infection with some appearing only in early phases and others in late phase (Dix et al., Journal of General Virology (1995), 76, 1051-1055). The encoded protein products of Orf1, Orf2, Orf3, Orf4, Orf6, and Orf6/7 were reported to exist in infected cells except for Orf3/4, which might be absent or expressed below detection limit (Täuber et al., Gene 278 (2001) 1-23). Orf1 encoded protein is expressed in the late phase and targets a family of cellular proteins that play a role in cell signaling and signal transfection. There is no functional information about E4 product encoded by Orf2. Furthermore, Ad5 mutants in which E4 Orf2 were deleted, were about to grow to wild-type levels (Täuber et al., Gene 278 (2001) 1-23). The deletion of Orf1 and 2 did not impact AAV production but improved its titer which indicated that E4 Orf1 and 2 are not essential (FIG. 5 of International Publication No. WO2023060113A). rAAV production titers were assessed using clone 1, 2, 4, and 6 HEK293-derived host cells.

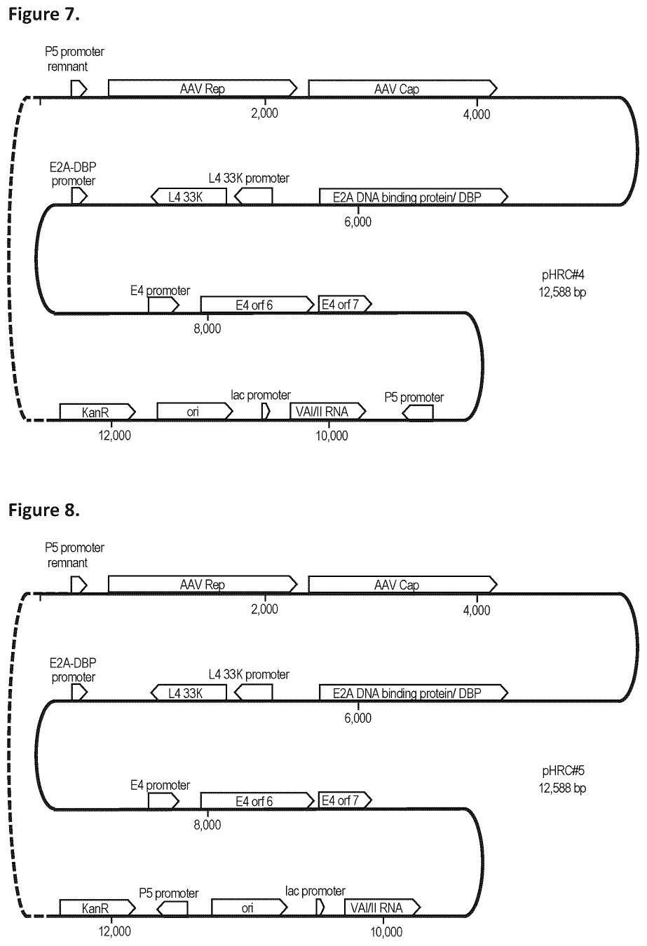

New helper plasmid #3 During helper plasmid #3 design, the E4 region was further dissected by sequential deletion. Different E4 variants with E4 native promoter and CMV promoter were screened for AAV production (FIG. 6 of International Publication No. WO2023060113A; wholeE4, E4Orf2+, E4Orf3+, E4Orf4+, E$Orf6/7 and E4Orf6 corresponds to helper plasmid #2, #3B, #3D, #3C, #3 and #3D, respectively). Those E4 variants with E4 Orf6-7 only gave the highest titers. E4 Orf3-4 was further removed from helper #2 to generate helper #3 (FIG. 7 of International Publication No. WO2023060113A). To further explain the rationale behind removing Orf3 and Orf4, it appears that Orf3 and Orf6 can partially or totally compensate for each other's defects. Orf3 and Orf6 have redundant functions and independently amplify viral DNA replication, late viral protein synthesis, shut-off of host protein synthesis, and prevent concatemer formation of viral genomes (Täuber et al., Gene 278 (2001) 1-23). E4 Orf4 also downregulates E4 transcription by inhibiting E1A-mediated transactivation of the E4 promoter through its interaction with the serine/threonine protein phosphatase 2A (PP2A), an enzyme that plays an important role on numerous cellular processes. This autoregulatory loop may be required to limit the cytotoxic effects of E4 gene products during the early phase of infection, where E4 Orf4 can induce apoptosis through caspase activation in a cell line-specific manner. Therefore, further removal of E4 Orf5 resulted in prevention of this cytotoxic effect (Tauber et al., Gene 278 (2001) 1-23).

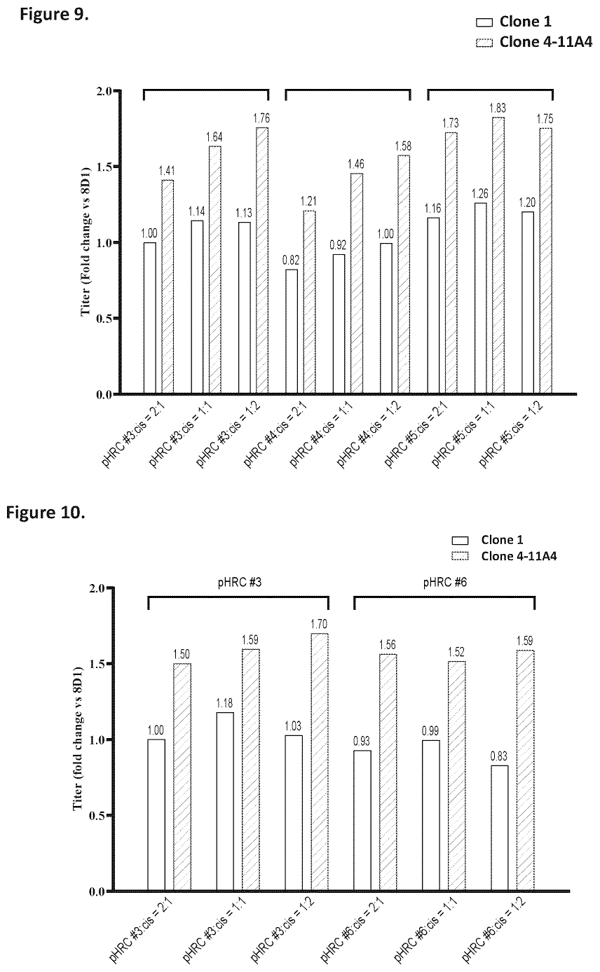

Helper #3 improved AAV titers including AAV8 and AAV9, and different transgenes (FIG. 8 of International Publication No. WO2023060113A) and product quality by improving % Full (FIG. 9 of International Publication No. WO2023060113A). rAAV production titers were assessed using clone 1 and 4 HEK293-derived host cells.

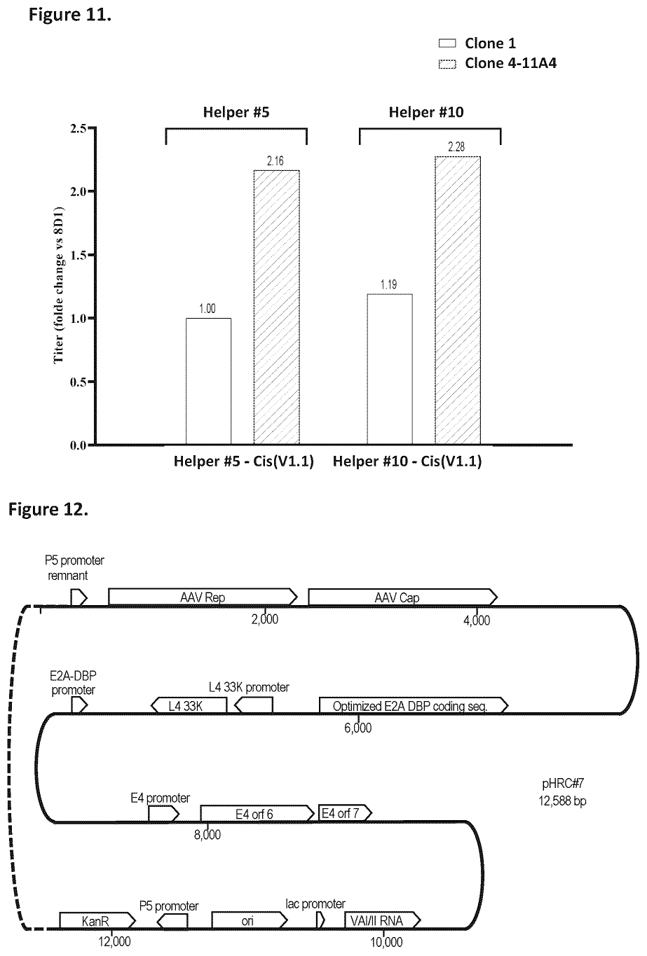

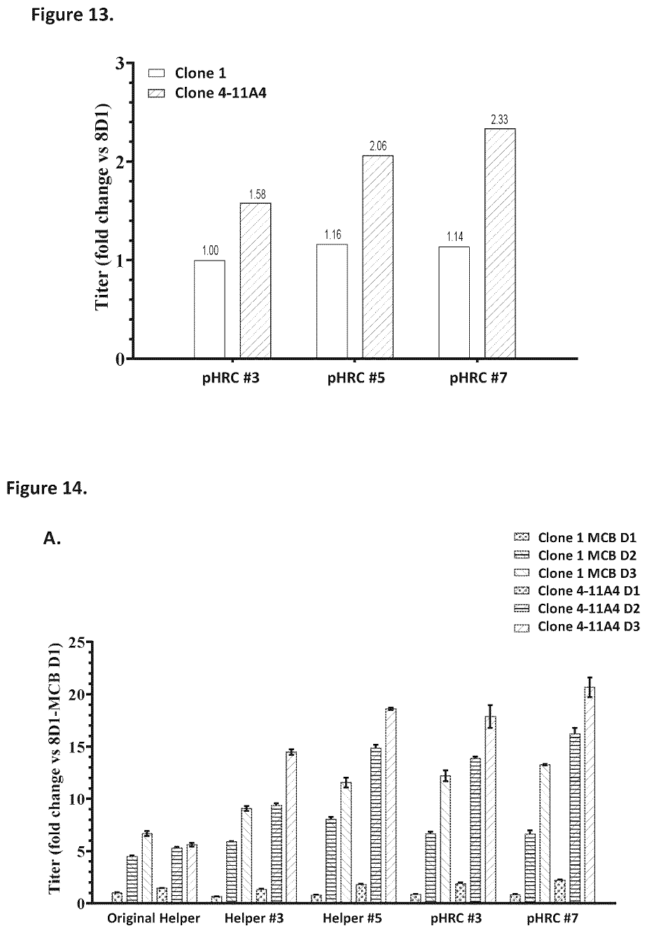

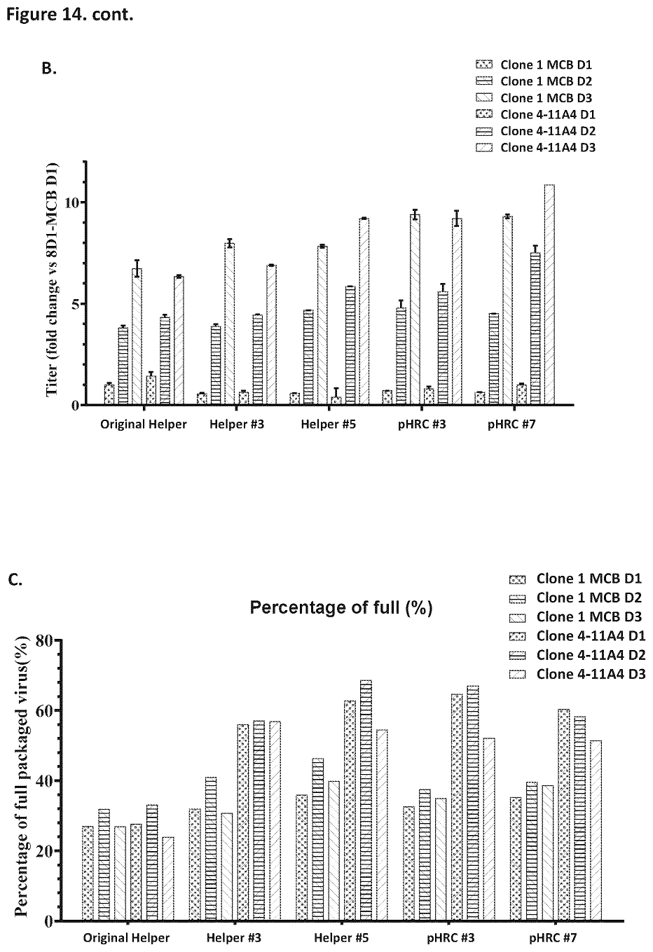

New helper plasmid #4 The possibility of adding other genes to the new helper plasmid to further improve AAV titers was investigated. Incorporation of selected genes from Boca virus helper that were reported to have positive impact on AAV production (Wang et al., Molecular Therapy: Methods & Clinical Development Vol. 11 Dec. 2018), addition of a copy of ELA gene and AAP (assembly-activating protein derived from trans plasmid) under CMV promoter were explored. The addition of Boca virus selected genes NP1 and NS2 genes to helper plasmid #2 (FIG. 10 of International Publication No. WO2023060113A) had no impact on AAV titers (FIG. 11 of International Publication No. WO2023060113A). It is known in the field that the assembly activating protein encoded by AAV capsid can provide increased capsid protein stability when expressed in trans (Maurer et al., 2018, Cell Reports 23, 1817-1830; Maurer et al., Journal Virology, 2019 Volume 93 Issue 7 e02013-18). The addition of AAP gene expressed in trans for AAV8 (FIG. 12 of International Publication No. WO2023060113A) had a negative impact on AAV titers (FIG. 14 of International Publication No. WO2023060113A). E1A is known to start AAV virus replication by enhancing the transcription from the rep gene promoters, P5 and P19 and by activating E2A and E4 adenovirus promoters. E1A is also known to control the host cell cycle to accommodate for AAV viral DNA replication. A potential drawback from overexpressing E1A is that it is known to stabilize p53, which can lead to apoptosis. This can be overcome by the E1B55K and the E4Orf6 proteins that will form a complex with p53 and cause it to be degraded (Matsushita et al., Journal of General Virology (2004), 85, 2209-2214; Meier et al., Viruses 2020, 12, 662). A copy of E1A under the control of CMV promoter was added to the helper plasmid #3 to create helper plasmid #4 (FIG. 13 of International Publication No. WO2023060113A). The location of E1A was between E4 and VA RNA I/II. The results indicated that this plasmid further improved AAV titers as shown in FIG. 14 of International Publication No. WO2023060113A. rAAV production titers were assessed using clone 1 and 4 HEK293-derived host cells.

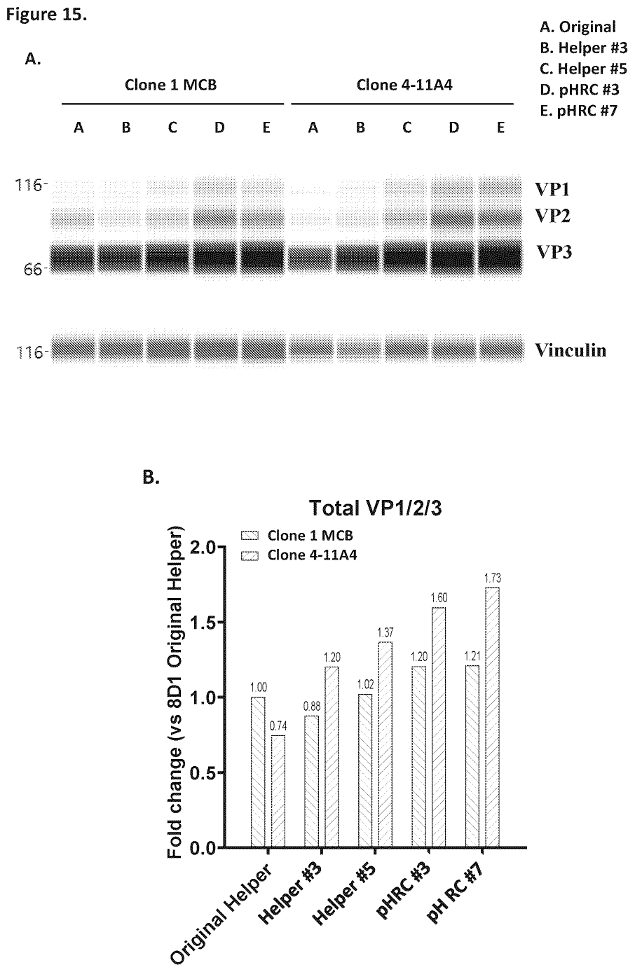

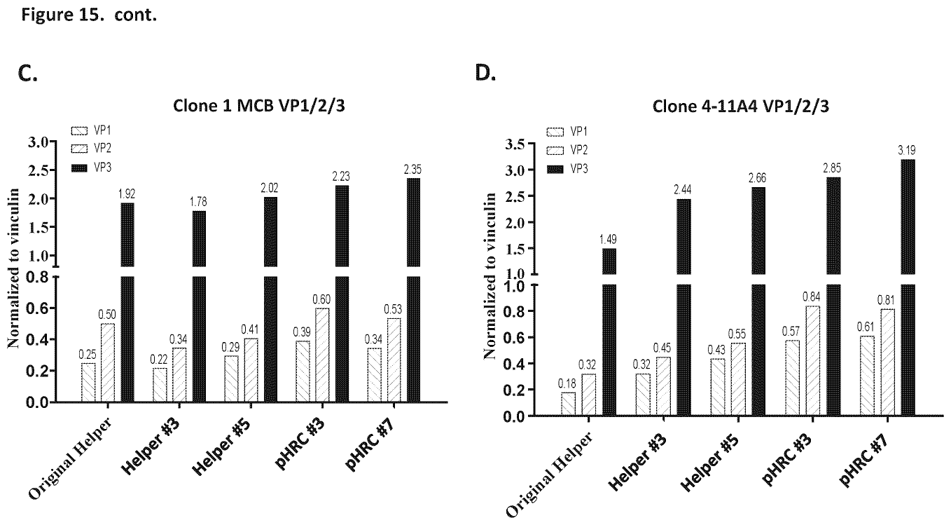

New helper plasmids #5, #6, #7, #8 and #9 It is known that E2A, E4 and VA RNA I/II microRNA are essential helper components for AAV production (Meier et al., Viruses 2020, 12, 662; doi: 10.3390/v12060662). In the current helper plasmids #1-4, L4 100K/hexon assembly and L4 22K/33K were kept in the helper plasmid #3 because their genes are located between the E2A promoter and E2A open reading frame. This region might be important since two E2A transcription starting sites (TSS) are located at this region as documented from the long-read direct RNA sequencing study of Donovan-Banfield et al., (Communication Biology (2020) 3:124). To test whether these two sequences could be removed while maintaining high titer, several mutations were generated based on helper #3 (Table 3). The analysis of all these mutations indicated that helper #5 and helper #8 gave similar titers or slightly higher titers than the helper plasmid #3 (FIG. 15 of International Publication No. WO2023060113A). rAAV production titers were assessed using clone 1 and 4 HEK293-derived host cells. In the helper plasmid #5, N-terminal region of encoded hexon assembly was removed, while in helper plasmid #8 the start codon was mutated for the hexon assembly region. On the other hand, all mutants in which L4 22K/33K start codon was mutated showed decrease in titers indicating that L4 22K/33K might be important for AAV production. These findings accord with the reported effect of L4 22K deletion, which resulted in continuous increase in E2A (DBP) expression in later phases and subsequently had a negative impact on E4 expression (Wu et al., Journal of Virology (2012) p.10474-10483; Guimet et al., Journal of Virology (2013) p.7688-7699). Helper plasmid #5A is identical to helper #5 except for not comprising a polynucleotide sequence of SEQ ID NO: 7 encoding E4 ORF7.

TABLE 3
Mutation of hexon assembly and L4 22K/33K
gene based on helper plasmid #3

| Plasmid | Size | | Hexon Assembly | L4 22K/33K |
|---|---|---|---|---|
| Helper #5 | 8.2 | Kb | Partial deletion | |
| Helper #6 | 8.2 | Kb | Partial deletion | Mutation (stop codon) |
| Helper #7 | 10.0 | Kb | | Mutation (stop codon) |
| Helper #8 | 10.0 | Kb | Mutation (stop codon) | |
| Helper #9 | 10 | Kb | Mutation (stop codon) | Mutation (stop codon) |

### Example 2. Development of Packaging Plasmids

pHRC Plasmids #1, #2 and #3

Three new packaging plasmids (pHRC #1, #2 and #3) were designed using the helper plasmids described herein. The sizes of the helper plasmids #3 and #5 described herein are only 10.0 and 8.2 Kb, respectively, which make the design of compact packaging plasmids feasible. See, PCT/US2022/077587, filed Oct. 5, 2022, and PCT/US2023/064500, filed Mar. 16, 2023, each of which is incorporated herein by reference in its entirety.

pHRC plasmid #1 was designed based on the helper plasmid #3 and a starting trans plasmid encoding AAV8 capsid. The rep and cap sequences of the starting trans plasmid were cloned between the KanR and E2A coding sequence of Helper #3 (FIG. 1). Similarly, pHRC plasmid #2 was designed based on the helper plasmid #5 and the starting trans plasmid. The rep and cap sequences of the starting trans plasmid were cloned between the KanR and E2A coding sequence of Helper #5 (FIG. 2). The Rep protein expression was controlled by a p5 promoter, which is around 3 Kb away from its start codon in the starting trans plasmid. After combining the rep and cap sequences with helper plasmid #5, the distance between the p5 promoter and the rep start codon changed from 3 Kb to 8.4 Kb in packaging plasmid #2. This long distance may weaken Rep protein expression. To address this possible problem, pHRC plasmid #3 was generated by moving the p5 promoter from its original location to a new site within the lac promoter. This change shortened the distance between the p5 promoter to the rep start codon from 8.4 Kb in pHRC #2 to 2.6 Kb in pHRC #3. (FIG. 3.)

The three packaging plasmids were evaluated by transient transfection using both Clone 1 and Clone 4-11A4 HEK293 cells. Transfection of two plasmids in order to produce AAV vectors that package a transgene, in this context, require the plasmid expressing rep, cap and helper genes (HRC plasmid, also called packaging plasmid) and a cis plasmid (carrying the genome expressing a transgene to be packaged). Since the optimal ratio between packaging plasmid and cis plasmid is not known, 3 different mass ratios that include 1:1, 1:0.1 and 3:0.1 packaging to cis were tested. Among all 3 constructs, pHRC #3 gave the highest titers of TG-X rAAV particles using the TG-X cis plasmid at 1:1 mass ratio (FIG. 4.)

To further confirm this result, a transient transfection was performed to produce TG-A and TG-B rAAV particles using clone 1, clone 4-11A4 and clone 4-9C4 HEK293 cells. For TG-A, different packaging to cis plasmid ratios were evaluated. FIG. 5. For TG-B, in addition to testing different packaging to cis plasmid ratios, different total mass of plasmids (m1, m2 and m3) were also tested. FIG. 6. As shown in FIG. 5, the 1:2 ratio of pHRC #3 to cis TG-A gave the highest titers. However, the optimal ratio for TG-B production was 2:1 ratio of pHRC #3 to cis TG-B. FIG. 6. For both cis plasmids, the two-plasmid system gave higher titers than the triple transfection system using the helper #5. These results indicated that the packaging to cis ratio needs to be optimized for each individual rAAV particle.

pHRC Plasmids #4 and #5

Since the initial results showed that the distance between the p5 promoter and the rep coding sequence significantly impacted the AAV titers, the distance was further optimized by designing pHRC #4 and pHRC #5 plasmids. The distance between the p5 promoter and the rep coding sequence in pHRC #4 and pHRC #5 is 3.6 Kb and 1.6 Kb, respectively (FIGS. 7 and 8). As shown in FIG. 9, pHRC #5 showed slight titer improvement compared to pHRC #3. This result indicated that moving the p5 promoter closer to the rep starting codon could improve the total virus expression, which might be due to the increased Rep protein expression levels.

Helper #10 and pHRC #6

The E2A and E4 codon sequences were originally adapted from the Adenovirus 5 gene. It is understood in the field that codon optimization of transgenes to maximize the codon usage of the expression host can improve expression levels of proteins encoded by a transgene. The E2A gene in Helper #5 was codon optimized to generate Helper #10; and the gene in pHRC #3 was optimized to generate pHRC #6. E4 optimized construct pHRC #6 resulted in a slight titer decrease for TG-A production (FIG. 10), while E2A optimized construct Helper #10 slightly improved TG-A titers (FIG. 11). Without being bound by any theory, codon optimization of E2A may have improved mRNA transcription, protein expression levels, and subsequently the stability of E2A protein.

pHRC Plasmid #7

Since the E2A optimized construct was based on Helper #5, to further evaluate its impact on the two-plasmid system, we replaced the E2A codon region in pHRC #5 with the optimized codon to generate pHRC plasmid #7 (FIG. 12). As shown in FIG. 13, pHRC #7 showed higher levels of TG-A titers than the other packaging plasmids tested.

### Comparison of Triple Transfection and Two-Plasmid Expression Systems

To compare the different expression systems generated, we performed a transient expression experiment using five different plasmids including the pAdDeltaF6 (original helper), Helper #3, Helper #5, pHRC #3 and pHRC #7 in clone 1 and Clone 4-11A4 HEK293 cells. As shown in FIG. 14 panel A, the newly designed two-plasmid system pHRC #3 and #8 provided similar or better titers than new helper plasmids #3 and #5. In addition, using new helpers #3 and #5 resulted in a slight increase in the total capsid titers. While a more dramatic capsid titer increase was observed in the case of the two-plasmid system pHRC #3 and pHRC #7, there was no significant capsid titer difference between the two cell lines tested (FIG. 14 panel B). Furthermore, using all newly designed plasmids, including the new helper #3, #5 and the two-plasmid system pHRC #3 and pHRC #7, during transient transfection resulted in an increase in % Full, particularly for the clone 4-11A4 (FIG. 14 panel C). % Full capsid was calculated based on the ddPCR and ELISA titers.

To better understand the mechanism of improved virus production by the two-plasmid system, Western blot analysis to determine expression levels of virus capsid proteins (VP1, VP2, VP3) and Replicase proteins (Rep78, Rep68, Rep52 and Rep40) was performed. As shown in FIG. 15 panel A, there were slightly more capsid proteins expressed in clone 4-11A4 compared to clone 1. Furthermore, the newly designed helper plasmids Helper #3 and Helper #5, and the packaging plasmids #3 and #8 improved total capsid expression levels in the clone 4-11A4. Using these new plasmids in clone 1 resulted in 1.2-fold improved capsid levels compared to the same clone with the original helper plasmid, while the capsid level improvement in the case of clone 4-11A4 using the same newly designed plasmids ranged from 1.2-1.7-fold (FIG. 15 panel B). The expression levels of individual capsid proteins VP1, VP2 and VP3 were also determined. As shown in FIG. 15 panel C and panel D, all of the virus capsid protein subunits VP1, VP2 and VP3 were increased in each newly designed construct in the clone 4-11A4, while this increase was not significant in clone 1.

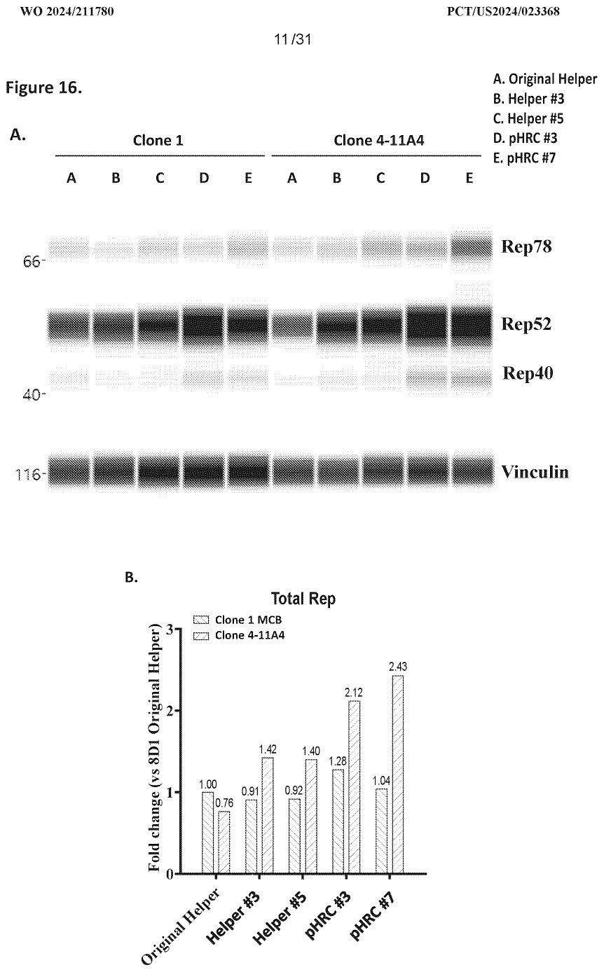

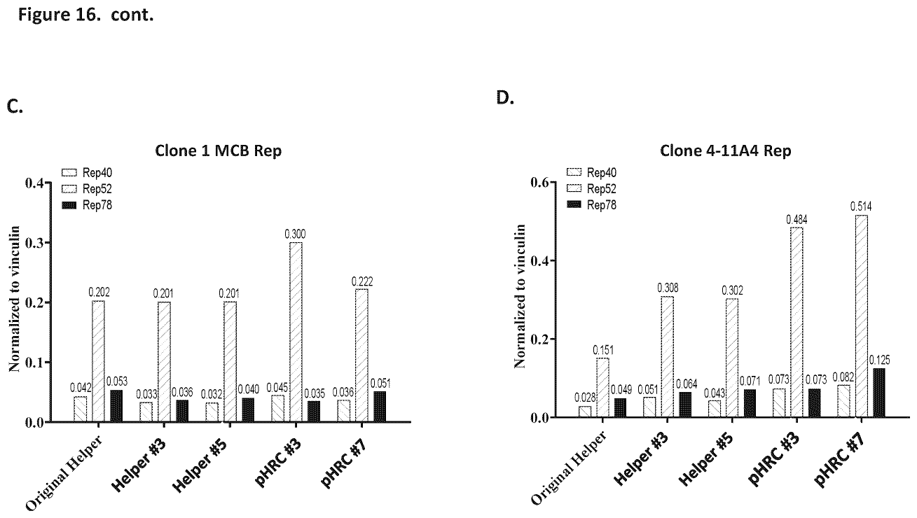

There are four Rep proteins, i.e., Rep78, Rep68, Rep52 and Rep40, encoded by the rep gene from the trans plasmid or packaging plasmid that result from alternative splicing of transcribed mRNA. However, only three Rep proteins, i.e., Rep78, Rep52 and Rep40, were observed in the Western blot. Without being bound by any theory, the lack of Rep68 detection might be due to its lower level of expression. Similar to the capsid Western blot results, there were more Rep proteins expressed in clone 2-11A4 compared to clone 1 (FIG. 16 panel A and panel B). Total Rep protein expression levels increased more significantly in the two-plasmid system using pHRC #3 and #7 (FIG. 16 panels B, C and D). Also, all the Rep protein subunits increased dramatically in clone 4-11A4 using pHRC #3 and #7 (FIG. 16 panel D).

Comparing the three-plasmid system using the original helper or the new helpers, and the two-plasmid system, the two-plasmid system described herein gave the highest AAV titers and improved % Full ~2-fold. It showed slight titer improvement and similar % Full compared to the triple plasmid system using the new helpers described herein. Without being bound by any theory, the titer increase observed for the two-plasmid system described herein can be due to improvement in both capsid and replicase protein expression levels, which could facilitate virus replication, capsid formation, and packaging, as observed in the capsid and Rep western blot results.

pHRC Plasmids #3, #5 and #7

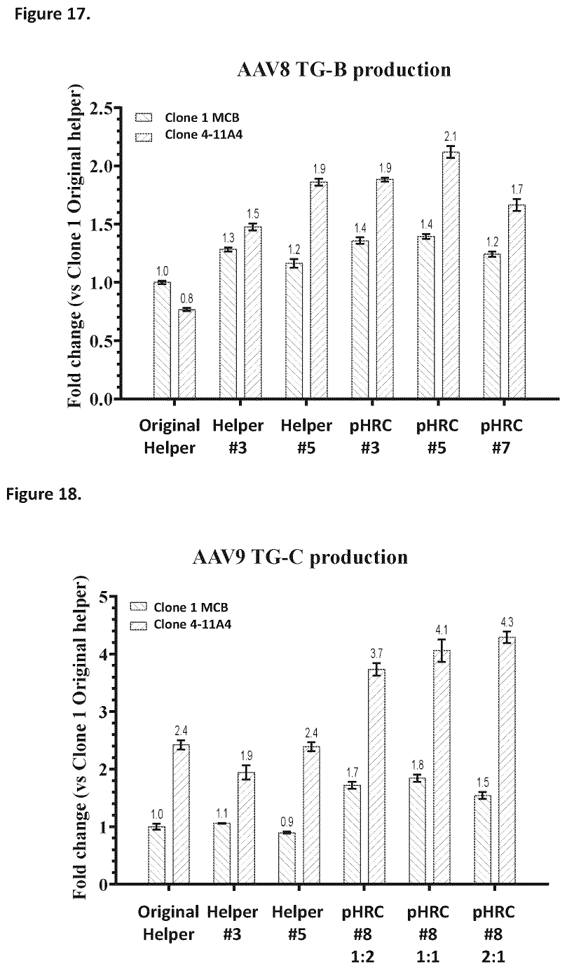

To test whether the two-plasmid system works well for different transgenes, TG-B rAAV8 particles were produced using the helper and packaging plasmids described herein. The Original helper, Helper #3, Helper #5, pHRC #3, pHRC #5, and pHRC #7 plasmids were tested. As shown in FIG. 17, all new helper and packaging plasmids tested significantly improved TG-B rAAV8 production in two different cell lines. The packaging plasmid pHRC #5 produced more viruses and gave 2-fold improvement compared to the level of production achieved by the original helper in Clone 1 cells.

pHRC Plasmid #8 Through #16

To confirm that the packaging plasmids described herein can be used to produce rAAV of all serotypes, packaging plasmids encoding AAV2, AAV6 and AAV9 capsids were produced by replacing the AAV8 capsid gene in pHRC packaging plasmids #3, #5 and #7 with sequences encoding AAV2, AAV6 or AAV9 capsids. The designation of the new packaging plasmids and their serotype is shown in Table 4. rAAV production by the AAV2 and AAV9 specific packaging plasmids was tested using the TG-A, TG-C and TG-D transgenes and 2 cell lines (Clone 2 and Clone 4-11A4). Results are shown in FIGS. 18-22.

TABLE 4
AAV8, AAV2, AAV6 and AAV9 packaging plasmids

| AAV8 | AAV2 | AAV6 | AAV9 |
|---|---|---|---|
| pHRC #3 | pHRC #12 | pHRC #13 | pHRC #11 |
| pHRC #5 | pHRC #15 | pHRC #16 | pHRC #14 |
| pHRC #7 | pHRC #9 | pHRC #10 | pHRC #8 |

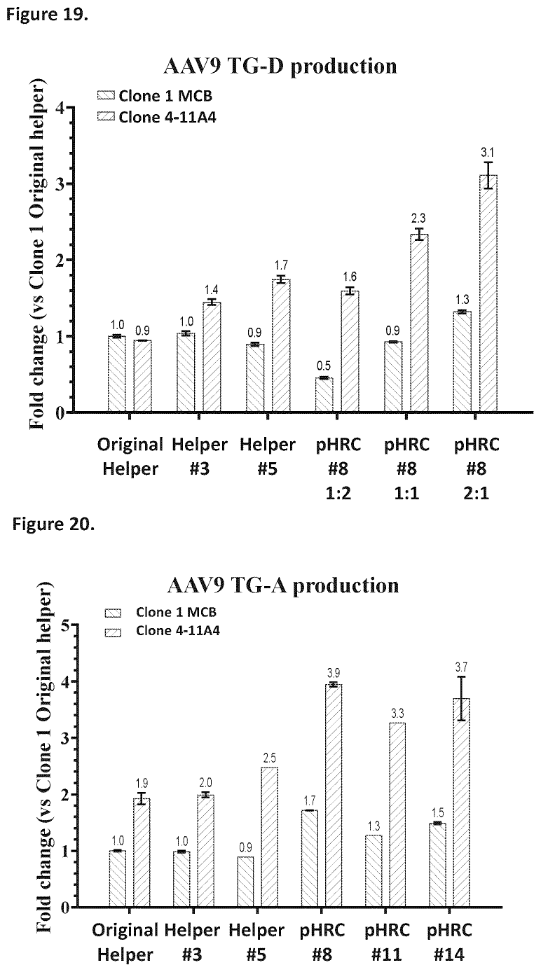

As shown in FIGS. 18 and 19, pHRC #8 used in Clone 4-11A4 cells increased TG-C and TG-D rAAV9 particle production by nearly four-fold and three-fold, respectively, compared to the titer achieved using the original helper in Clone 1 cells. Packaging plasmid to transgene plasmid ratios of 1:2, 1:1 and 2:1 were tested for pHRC #8.

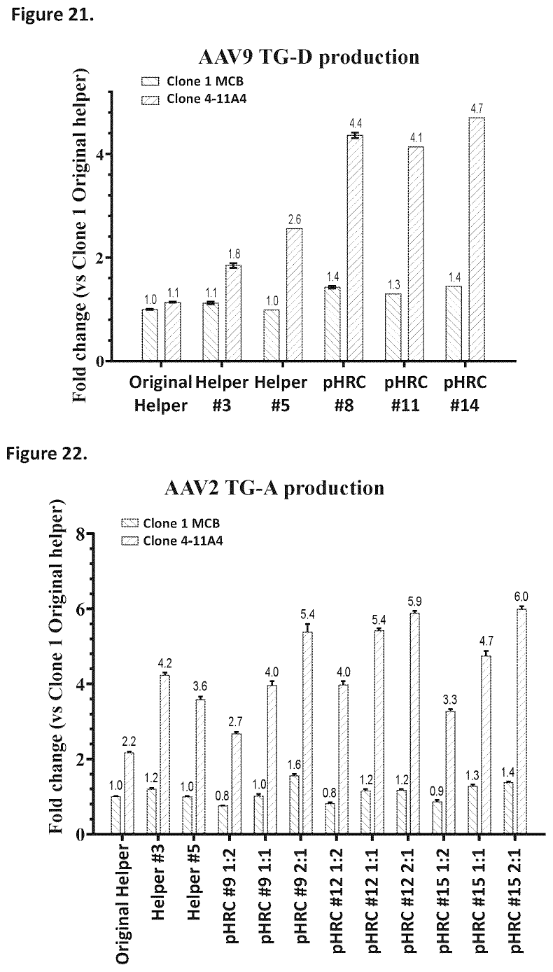

As shown in FIGS. 20 and 21, the efficiency of the pHRC #8, #11 and #14 rAAV9 packaging plasmids was compared by testing the three plasmids ability to facilitate the production of TG-A and TG-D rAAV9 particles. Overall, no significant differences were observed between the three plasmids. When used with Clone 4 11A4 cells, all these plasmids gave a 3-4-fold increase compared to the titer achieved using the original helper in Clone 1 cells.

As shown in FIG. 22, the pHRC #9, #12 and #15 AAV2 specific packaging plasmids were similarly evaluated for TG-A rAAV2 production. Packaging plasmid to transgene plasmid ratios of 1:2, 1:1 and 2:1 were tested. pHRC #12 and #15 gave slightly higher titers than pHRC #9. In Clone 4 11A4 cells, pHRC #9, #12 and #15 showed 67% titer increase compared to Helper #5. pHRC #9, #12 and #15 used in Clone 4 11A4 cells gave a 6-fold improvement compared to the original helper used in Clone 1 cells. And in Clone 1 cells, pHRC #9, #12 and #15 gave a ~3-fold improvement compared to the original helper.

### Bioreactor Study

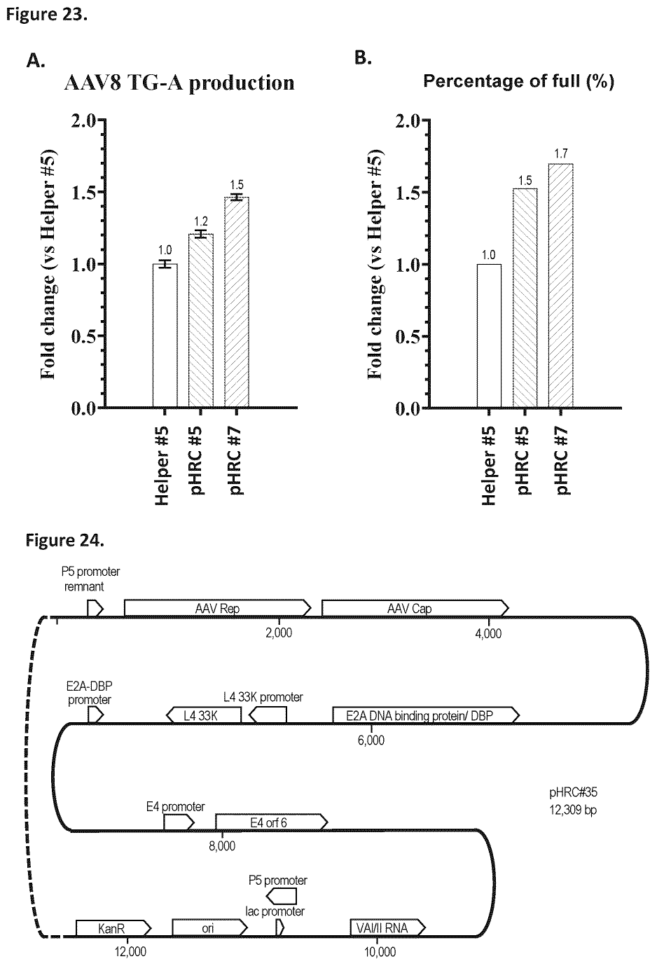

The packaging plasmids described herein gave significant titer improvement over the three-plasmid system in small scale experiments. To confirm that the packaging systems are suitable for scale-up and to check the product quality, a 5-liter bioreactor study was performed to compare the pHRC #5 and #7 AAV8 packaging plasmids with Helper #5 for TG-A rAAV8 production. As shown in FIG. 23A, pHRC #5 and #7 improved rAAV titers by 1.2- and 1.5-fold, respectively. Viral titers were measured using ddPCR. To check product quality of harvest materials, one step affinity purification was performed followed by AUC (Analytical Ultracentrifugation). As shown in FIG. 23B, pHRC #5 and #7 gave much higher % Full, as determined by AUC, than Helper #5. pHRC #7 gave the overall best product quality with a 1.7-fold improvement in % full particles over Helper #5.

### E4 ORF6

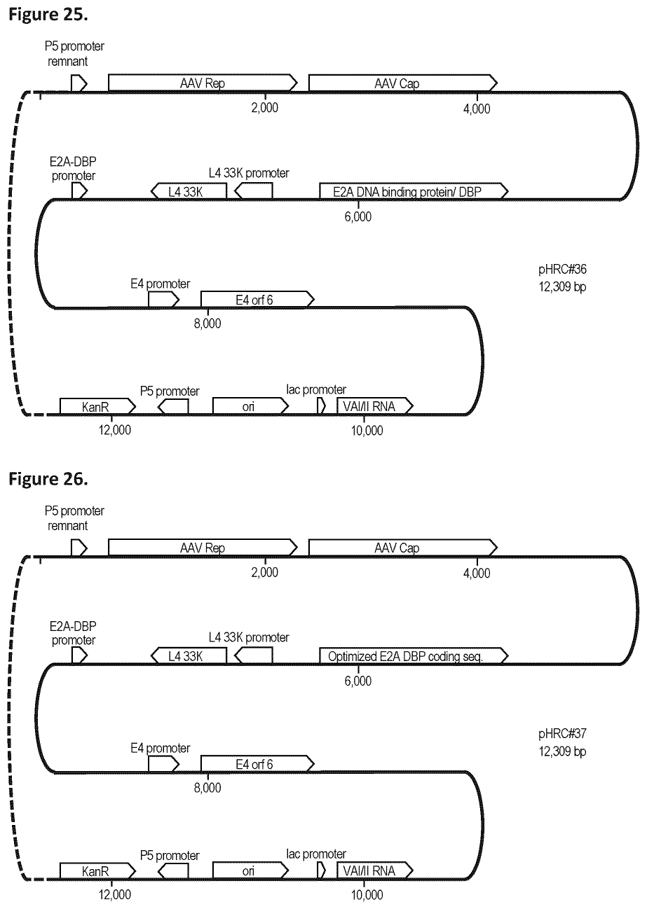

To confirm that E4 ORF6 alone is sufficient to mediate rAAV particles using the packaging plasmids described herein, pHRC #35, #36 and #37 packaging plasmids were produced by replacing the nucleotide sequence encoding E4 ORF6 and 7 in pHRC #3, #5 and #7, respectively, with a nucleotide sequence encoding E4 ORF6. A map representing pHRC #35, #36 and #37 packaging plasmid is shown in FIGS. 24-26, respectively. The efficiency of pHRC #35, #36 and #37 to produce rAAV will be evaluated.

pHCR #17 Through #34-Membrane-Associated Accessory Protein

Membrane-associated accessory protein (mAAP), a polypeptide encoded by AAV, has been linked to virus secretion. See, e.g., Ogden et al., Comprehensive AAV capsid fitness landscape reveals a viral gene and enables machine-guided design, Science, 2019 Nov. 29, 366(6469):1139-1143. Truncation of mAAP has been reported to improve the virus titers. See, e.g., Galibert et al., Functional roles of the membrane-associated AAV protein MAAP, Sci Rep. 2021 Nov. 4, 11(1):21698. To evaluate the impact of mAAP truncation on rAAV titers in the context of the packaging plasmids described herein, AAV2, AAV8 and AAV9 packaging plasmids comprising one of 6 different mAAP non-sense mutations were produced by modifying the pHRC #15, #7 and #8 starting plasmids, as listed in Table 5.

TABLE 5
mAAP mutant packaging plasmids

| mAAP mutation | AAV2 | AAV8 | AAV9 |
|---|---|---|---|
| Wild type starting plasmid | pHRC #15 | pHRC #7 | pHRC #8 |
| S39->Stop | pHRC #17 | pHRC #23 | pHRC #29 |
| L78->Stop | pHRC #18 | pHRC #24 | pHRC #30 |
| E90->Stop | pHRC #19 | pHRC #25 | pHRC #31 |
| L100->Stop | pHRC #20 | pHRC #26 | pHRC #32 |
| L106->Stop | pHRC #21 | pHRC #27 | pHRC #33 |
| L110->Stop | pHRC #22 | pHRC #28 | pHRC #34 |

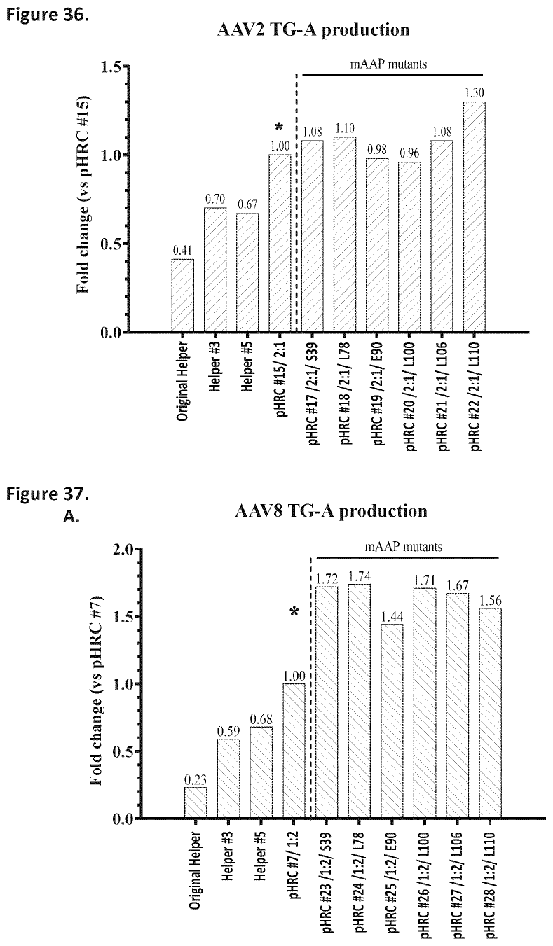

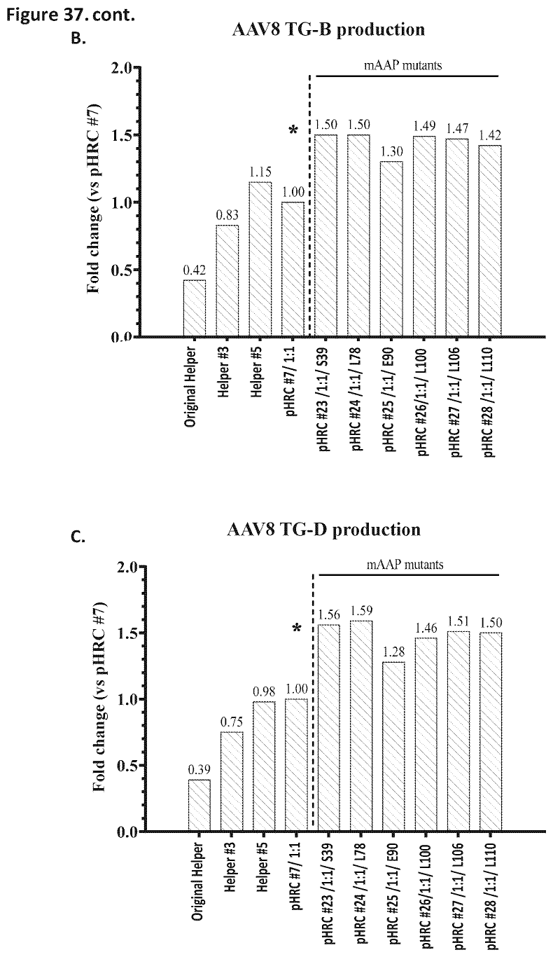

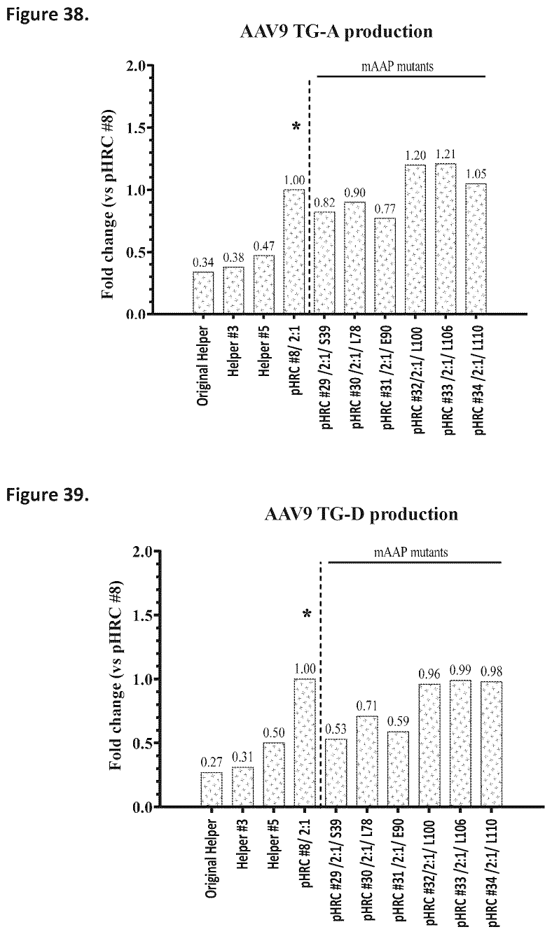

As shown in FIGS. 36-39, a non-sense mutation in mAAP improved rAAV virus titers in several serotypes. For example, rAAV titer increased 1.3-fold for TG-A rAAV2 (FIG. 36), 1.7-fold for TG-A rAAV8 (FIG. 37A) and 1.2-fold for TG-A rAAV9 (FIG. 38). Since the most significant improvement was observed for AAV8 serotype, two more transgenes expressed in AAV8 capsids were tested (FIGS. 37B and C). Titer improvement was observed for both TG-B rAAV8 and TG-D rAAV8 (FIGS. 37B and C). The TG-D transgene tested in the context of AAV9 did not show titer improvement (FIG. 39).

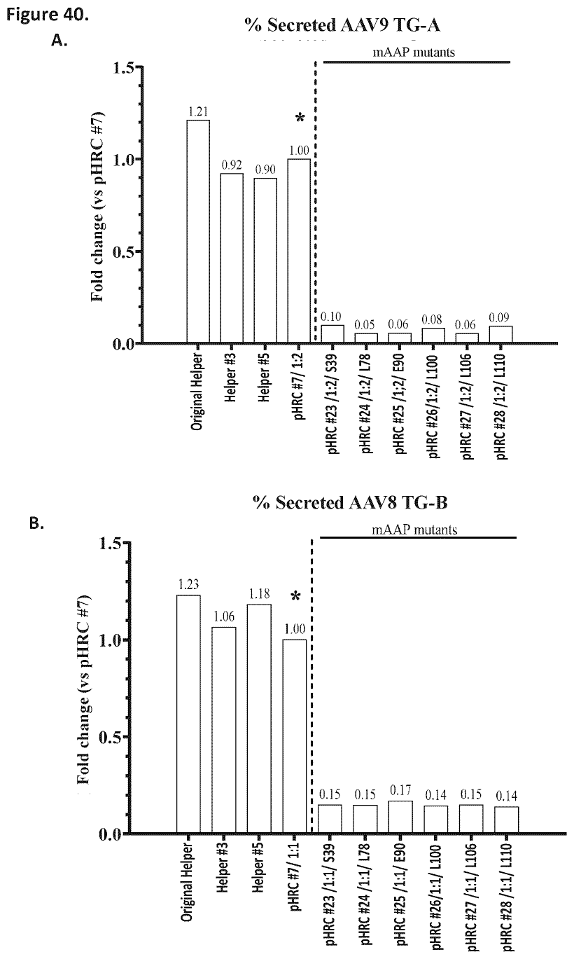

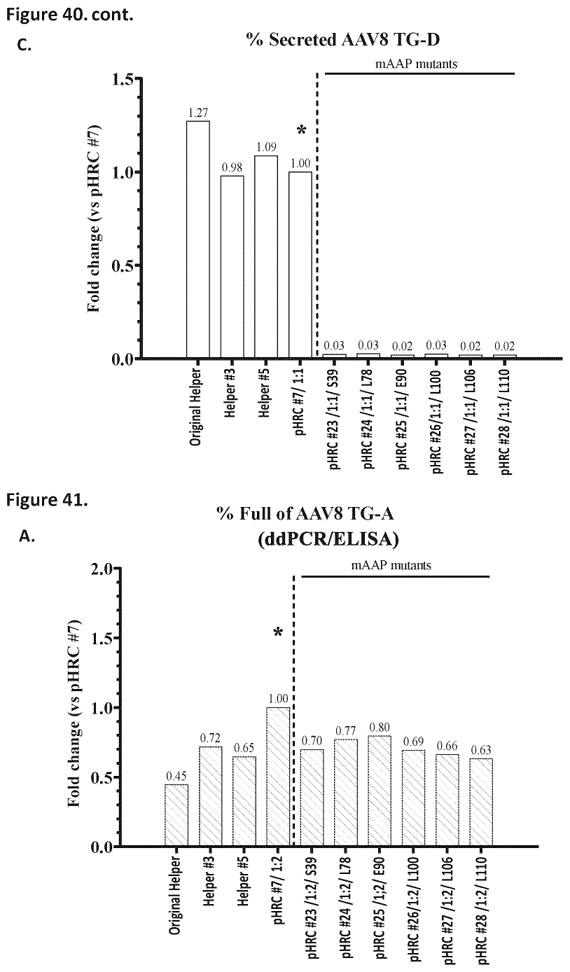

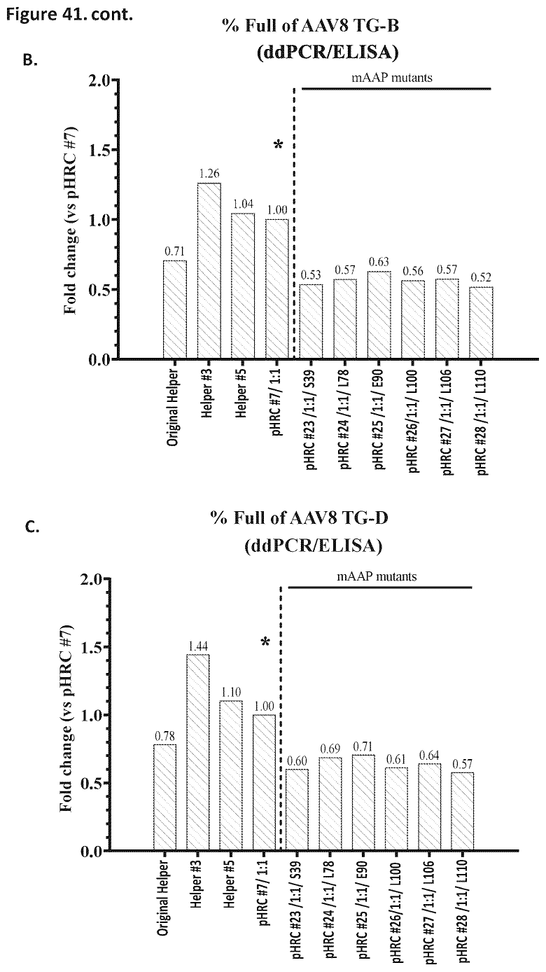

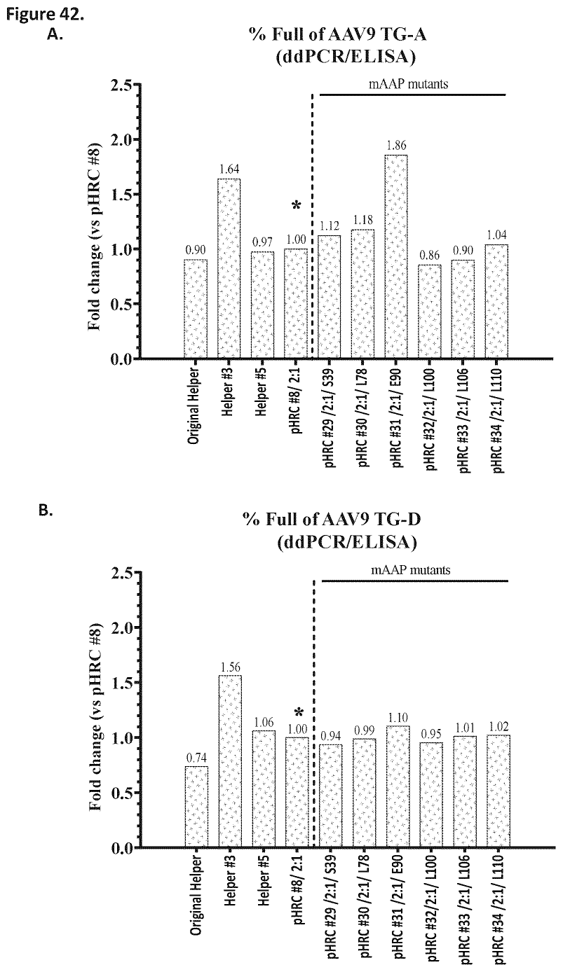

As shown in FIG. 40, mAAP mutations significantly lowered the percentage of rAAV8 viruses secreted into the medium, as reported previously. See, e.g., Galibert L, et al., Functional roles of the membrane-associated AAV protein MAAP, Sci Rep. 2021 Nov. 4, 11 (1): 21698. (FIG. 40). The % Full of rAAV8 viruses also decreased for all the tested transgenes (FIG. 41). For serotype AAV9, the impact of mAAP mutations on virus quality, as reflected by % full, was transgene specific (FIG. 42). One of six mutations tested (E90 to stop codon) increased the total % Full for TG-A rAAV9, whereas all 6 mAAP mutations tested decreased % Full of TG-D rAAV9.

For all six mAAP mutations incorporated into pHRC #17 through #34, mAAP was partially expressed. It has been reported that complete removal of mAAP expression could also boost titers of AAV1, AAV8 and AAV9. See, e.g., Elmore et al., The membrane associated accessory protein is an adeno-associated viral egress factor, Nat Commun. 2021 Oct. 29, 12 (1): 6239. pHRC #38 and pHRC #39 were created by incorporating a mAAP modified start codon (ctg to cag) mutation to further test whether ablation of mAAP can increase rAAV production utilizing the two-plasmid system. The efficiency of pHRC #38 and #39 to produce rAAV will be evaluated.

### Example 3. Development of a One-Plasmid Expression System

Given the significant titer improvement provided by the two-plasmid system of rAAV production using the packaging plasmids described herein compared to the three-plasmid triple transfection system, a one-plasmid transfection system was developed to test whether it can further increase rAAV titers and product quality. In the past, developing a single plasmid with all the components required for rAAV production that is suitable for large scale, GMP compliant manufacturing would have been extremely challenging, if at all possible, due to the large size of the plasmid. Combining the necessary parts of the known helper, trans and cis plasmids into a single molecule would have resulted in a plasmid larger than 25-30 Kb. Such large size would have made GMP plasmid manufacture process very challenging. In addition, the large plasmid size would have lowered transfection efficiency resulting in lower rAAV virus titers. Until now, no research team reported the use of a one-plasmid system for transient transfection to produce rAAV.

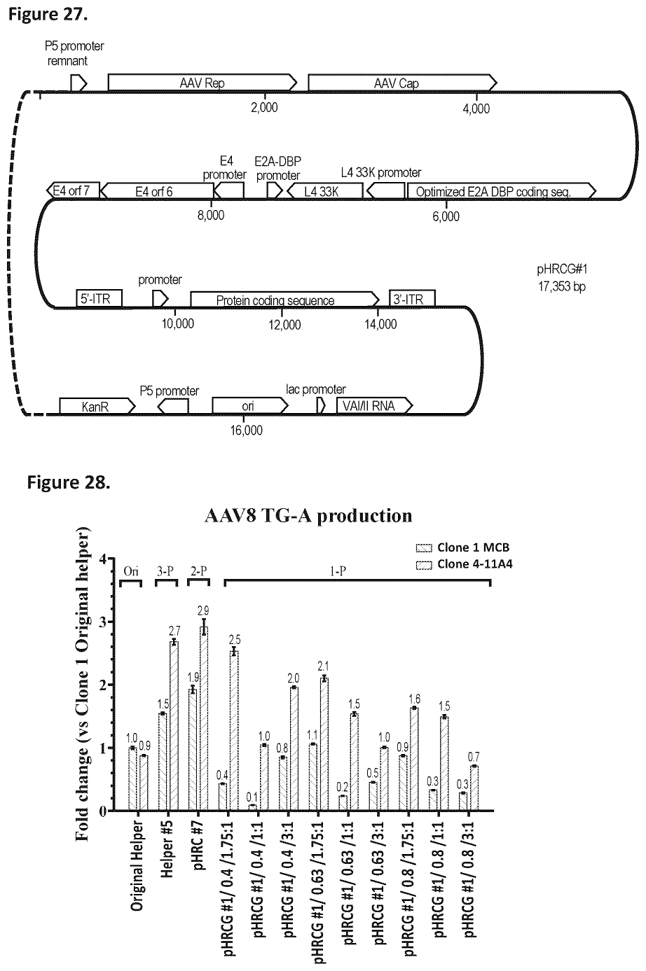

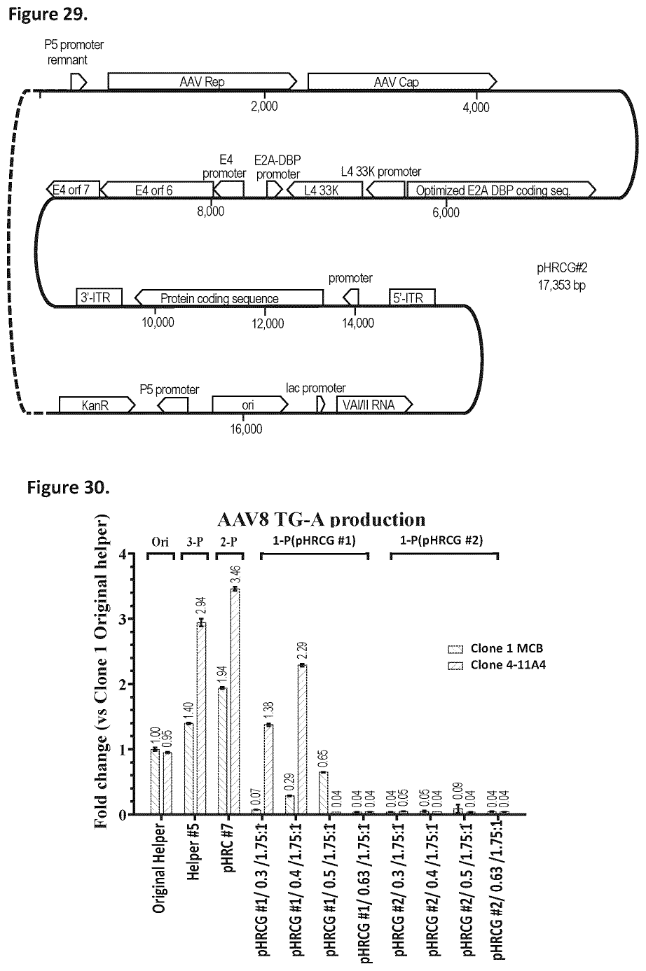

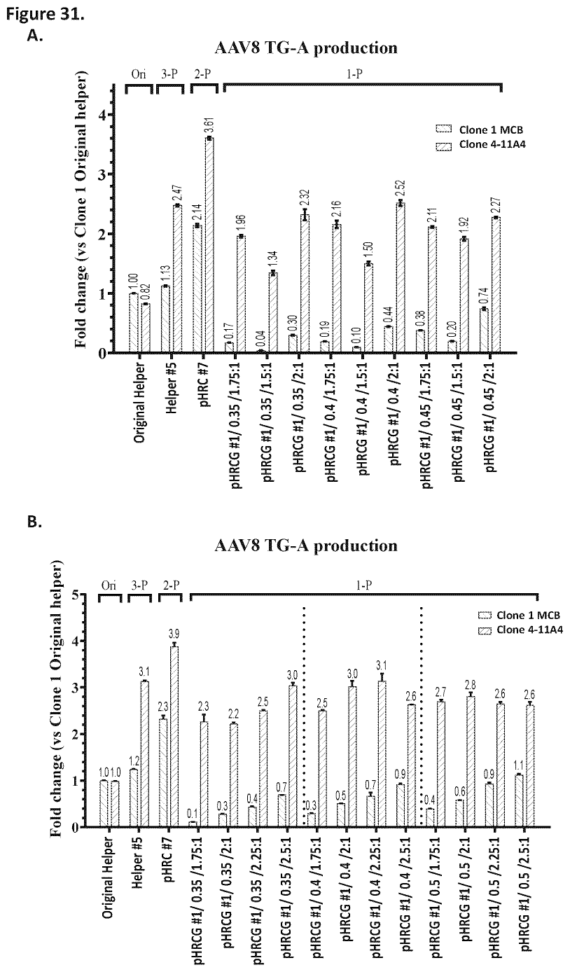

Given that the Helper #5 plasmid described herein at 8.2 Kb is only about half as large as the stating Original helper plasmid, and given that the new packaging plasmids were only about 12.6 Kb, designing a one-plasmid system for rAAV production became feasible. The pHRCG #1 complete plasmid was produced by inserting into the pHRC #7 packaging plasmid a fragment encoding the TG-A cis-transgene with its flanking ITRs into the region between VARNA and E4 sequence of pHRC #7 (FIG. 27). The TG-A rAAV8 titer produced by pHRCG #1 complete plasmid using different amounts of total DNA and PEI: plasmid ratios are shown in FIG. 28. Under some conditions, the titer produced by pHRCG #1 surpassed the titer produced using the original helper and was comparable to the titers produced by Helper #5 and pHRC #7. To evaluate the impact of ITR-transgene orientation on production titers, the pHRCG #2 complete plasmid was produced in which the fragment encoding the TG-A cis-transgene with its flanking ITRs is inserted at the same site as in pHRCG #1 but in opposite orientation compared to pHRCG #1 (FIG. 29). Surprisingly, pHRCG #2 produced significantly lower rAAV titers than pHRCG #1 (FIG. 30). These results indicated that the orientation of ITR-transgene is critical for virus replication and packaging. Several DOE studies were performed to optimize the total amount of DNA and PEI/DNA ratio for improving TG-A rAAV8 titers produced by the pHRCG #1 complete plasmid (FIGS. 31A &B). The optimal process gave titers close to the titers produced by the two-plasmid system using the pHRC #7 packaging plasmid.

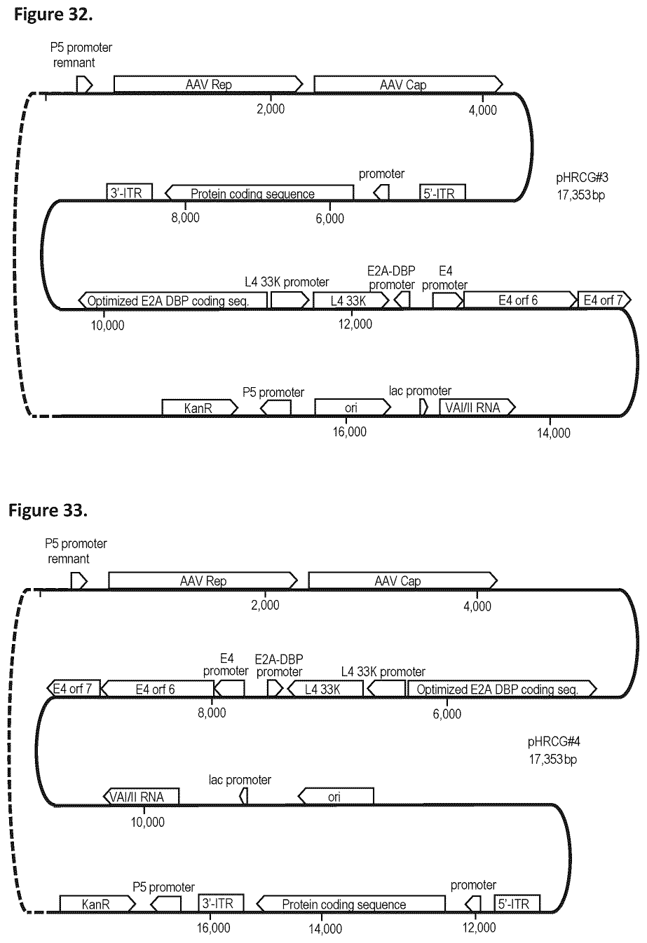

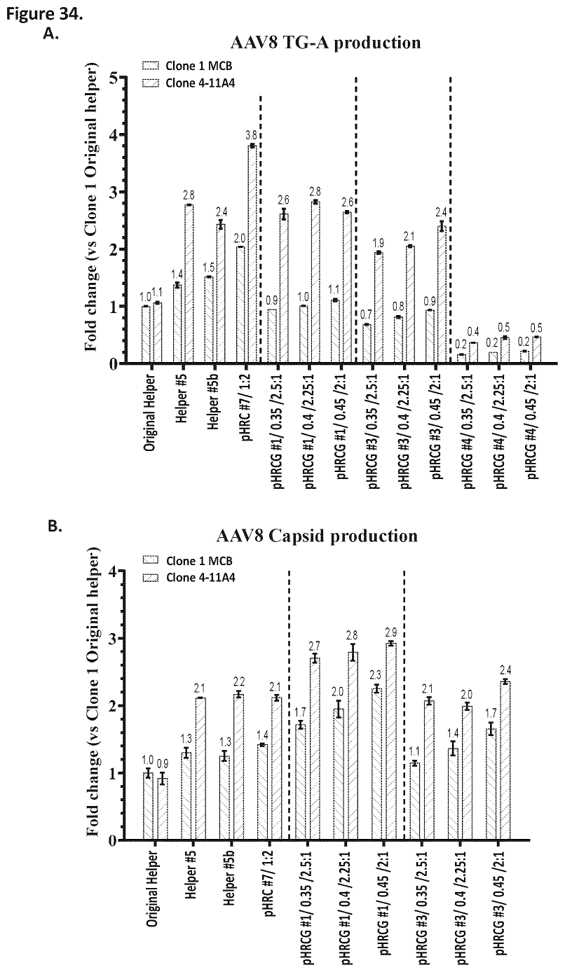

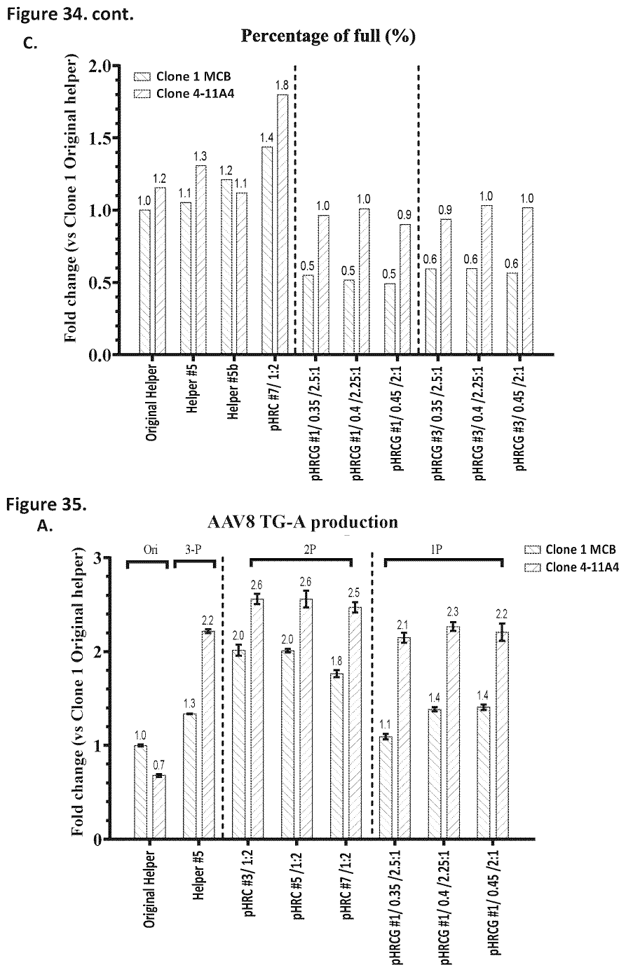

To further improve the one-plasmid system disclosed herein, pHRCG #3 and pHRCG #4 complete plasmids were produced by inserting the fragment encoding the TG-A cis-transgene with its flanking ITRs close to the capsid encoding sequence or the replicase encoding sequence, respectively, of the pHRC #7 packaging plasmid. Maps representing the pHRCG #3 and pHRCG #4 complete plasmids are shown in FIGS. 32 and 33, respectively. Moving the ITR-transgene close to capsid sequence in pHRCG #3 did not improve virus titers, whereas moving the ITR-transgene close to replicase sequence in pHRCG #4 gave significantly lower virus titers (FIG. 34A). rAAV particles produced by using pHRCG #1 were characterized by a % Full value, determined by ddPCR and ELISA, that was lower than the % Full value of rAAV particles produced using the pHRC #7 (FIG. 34A). Without being bound by any theory, the lower % Full virus value can be due to the increased capsid concentrations detected in cells transfected with pHRCG #1 (FIG. 34B).

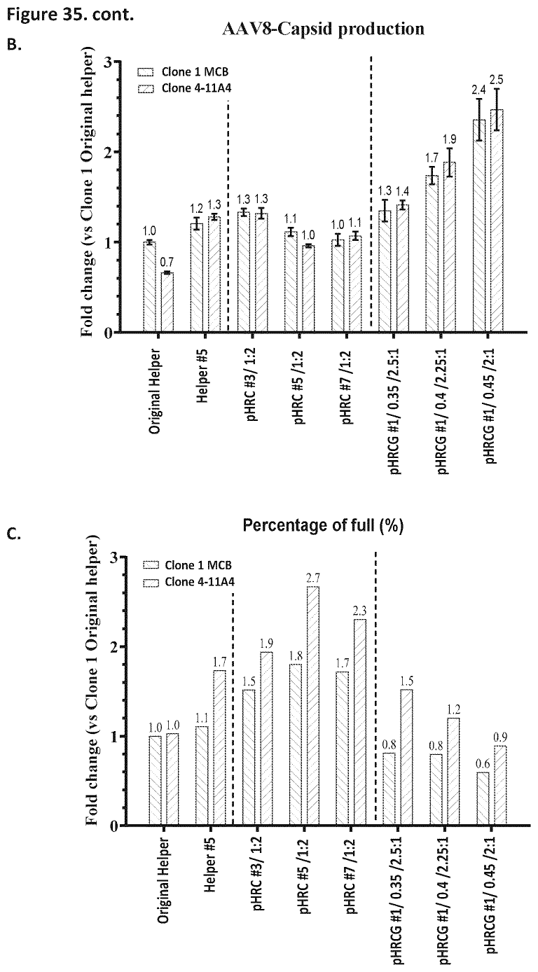

TG-A rAAV8 production using the original helper, Helper #5, pHRC #7 and pHRCG #1 in Clone 1 and Clone 4-11A4 cells was compared (FIG. 35). Helper #5 improved titers and % Full compared to the original helper plasmid. The two-plasmid system using the pHRC #7 packaging plasmid further improved total rAAV yield and quality, as reflected by % Full, compared to the three-plasmid system using Helper #5. And the one-plasmid system using the pHRCG #1 complete plasmid gave similar titers as the two-plasmid system using pHRC #7 but reduced product quality.

In conclusion, the two-plasmid system using packaging plasmids described herein significantly improves rAAV titer and quality, as reflected by % Full, compared to the three-plasmid system using helper plasmids. Without being bound by any theory, the titer increase observed using the two-plasmid system can be due to increase in both capsid and replicase protein expression levels, which would facilitate virus replication, capsid formation, and packaging, as shown in the capsid and Rep western blot results. The two-plasmid system using packaging plasmids described herein has shown great potential to replace the triple transfection system currently used in rAAV manufacturing.

While the disclosed methods have been described in connection with what is presently considered to be the most practical and preferred embodiments, it is to be understood that the methods encompassed by the disclosure are not to be limited to the disclosed embodiments, but on the contrary, is intended to cover various modifications and equivalent arrangements included within the spirit and scope of the appended claims.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

1. An isolated recombinant polynucleotide comprising
a) a first nucleotide sequence encoding an adenovirus E2A DNA binding protein (DBP) operably linked to a first promoter;
b) a second nucleotide sequence encoding an adenovirus E4 polypeptide operably linked to a second promoter;
c) a third nucleotide sequence encoding an adenovirus VA RNA I; and
d) a fourth nucleotide sequence encoding a parvovirus p5 promoter, which is optionally an adeno associated virus (AAV) p5 promoter, and a fifth nucleotide sequence encoding an AAV rep gene and an AAV cap gene, wherein the parvovirus p5 promoter is operably linked to the AAV rep gene and controls the expression of the rep78 and rep68 gene products,
optionally wherein the isolated recombinant polynucleotide does not comprise a nucleotide sequence encoding an adenovirus ITR sequence, L3 23K endoprotease, L5 pVI/fibre, and/or L4 pVIII/hexon-associated precursor.

2. The isolated recombinant polynucleotide of claim 1, wherein
a) the fourth nucleotide sequence encodes an AAV p5 promoter, which is positioned between 1,000 and 2,000 nucleotides upstream from the AAV rep start codon, wherein the isolated recombinant polynucleotide comprises
a1) a fragment comprising the first nucleotide sequence encoding an adenovirus E2A DBP and the second nucleotide sequence encoding an adenovirus E4 ORF6 polypeptide, wherein the fragment comprises a nucleotide sequence having at least 95% identity to SEQ ID NO: 282,
a2) a fragment comprising the first nucleotide sequence encoding an adenovirus E2A DBP, the second nucleotide sequence encoding an adenovirus E4 ORF6 polypeptide and the third nucleotide sequence encoding an adenovirus VA RNA I and VA RNA II, wherein the fragment comprises a nucleotide sequence having at least 95% identity to SEQ ID NO: 158,
a3) a fragment comprising the first, second, third, and fifth nucleotide sequences, wherein the fragment comprises a nucleotide sequence having at least 95% identity to SEQ ID NO: 161, or
a4) a nucleotide sequence having at least 95% identity to SEQ ID NO: 164;
b) the fourth nucleotide sequence encodes an AAV p5 promoter, which is positioned between 1,000 and 2,000 nucleotides upstream from the AAV rep start codon, wherein the isolated recombinant polynucleotide comprises
b1) a fragment comprising the first nucleotide sequence encoding an adenovirus E2A DBP and the second nucleotide sequence encoding an adenovirus E4 ORF6 polypeptide, wherein the fragment comprises a nucleotide sequence having at least 95% identity to SEQ ID NO: 281,
b2) a fragment comprising the first nucleotide sequence encoding an adenovirus E2A DBP, the second nucleotide sequence encoding an adenovirus E4 ORF6 polypeptide and the third nucleotide sequence encoding an adenovirus VA RNA I and VA RNA II, wherein the fragment comprises a nucleotide sequence having at least 95% identity to SEQ ID NO: 157,
b3) a fragment comprising the first, second, third, and fifth nucleotide sequences, wherein the fragment comprises a nucleotide sequence having at least 95% identity to SEQ ID NO: 160, or
b4) a nucleotide sequence having at least 95% identity to SEQ ID NO: 163; or
c) the fourth nucleotide sequence encodes an AAV p5 promoter, which is positioned between 2,000 and 3,000 nucleotides upstream from the AAV rep start codon, wherein the isolated recombinant polynucleotide comprises
c1) a fragment comprising the first nucleotide sequence encoding an adenovirus E2A DBP and the second nucleotide sequence encoding an adenovirus E4 ORF6 polypeptide, wherein the fragment comprises a nucleotide sequence having at least 95% identity to SEQ ID NO: 280,
c2) a fragment comprising the first nucleotide sequence encoding an adenovirus E2A DBP, the second nucleotide sequence encoding an adenovirus E4 ORF6 polypeptide and the third nucleotide sequence encoding an adenovirus VA RNA I and VA RNA II, wherein the fragment comprises a nucleotide sequence having at least 95% identity to SEQ ID NO: 156,
c3) a fragment comprising the first, second, third, and fifth nucleotide sequences, wherein the fragment comprises a nucleotide sequence having at least 95% identity to SEQ ID NO: 159, or
c4) a nucleotide sequence having at least 95% identity to SEQ ID NO: 162.

3. The isolated recombinant polynucleotide of claim 1, wherein the nucleotide sequence encoding the adenovirus E2A DBP and the nucleotide sequence encoding the adenovirus E4 polypeptide are in opposite 5′ to 3′ orientation.

4. The isolated recombinant polynucleotide of claim 1 characterized by one or more of:
a) the adenovirus E2A DBP comprises an amino acid sequence having at least 90% identity to SEQ ID NO: 45,
b) the E4 polypeptide comprises the E4 ORF6 and ORF7, optionally wherein the adenovirus E4 ORF6 polypeptide comprises an amino acid sequence having at least 90% identity to SEQ ID NO: 46 and the adenovirus E4 ORF7 polypeptide comprises an amino acid sequence having at least 90% identity to SEQ ID NO: 120,
c) the E4 polypeptide comprises the E4 ORF6, optionally wherein the adenovirus E4 ORF6 polypeptide comprises an amino acid sequence having at least 90% identity to SEQ ID NO: 46,
d) wherein the nucleotide sequence encoding the adenovirus VA RNA I comprises a nucleotide sequence having at least 90% identity to SEQ ID NO: 54, and
e) wherein the nucleotide sequence encoding the adenovirus VA RNA I encodes VA RNA I and VA RNA II, and optionally comprises a nucleotide sequence having at least 90% identity to SEQ ID NO: 9.

5. The isolated recombinant polynucleotide of claim 1 characterized by one or more of:
a) the first promoter and second promoter are different promoters,
b) the first promoter is an adenovirus E2A promoter, and
c) the second promoter is an adenovirus E4 promoter.

6. The isolated recombinant polynucleotide of claim 1 characterized by one or more of:
a) the isolated recombinant polynucleotide comprises a nucleotide sequence encoding the E2A promoter, L4 22K/33K polypeptides and promoter, L4 100 k/hexon assembly polypeptide comprising an N terminal deletion and the E2A DBP, wherein the N-terminal deletion of the L4 100 k/hexon assembly polypeptide corresponds to the nucleotide sequence of SEQ ID NO: 21, and optionally wherein the nucleotide sequence has at least 90% identity to SEQ ID NO: 22,
b) the isolated recombinant polynucleotide comprises a nucleotide sequence encoding the E2A promoter, L4 22K/33K polypeptides and promoter, L4 100 k/hexon assembly polypeptide comprising a mutation in its start codon and the E2A DBP, optionally wherein the nucleotide sequence has at least 90% identity to SEQ ID NO: 23,
c) the isolated recombinant polynucleotide comprises a nucleotide sequence encoding the E2A promoter, L4 22K/33K polypeptides and promoter, L4 100 k/hexon assembly polypeptide comprising an N terminal deletion and the E2A DBP, wherein the N-terminal deletion of the L4 100 k/hexon assembly polypeptide encompasses the start codon of L4 100 k/hexon assembly but does not encompass the start codon of the L4 22K/33K polypeptides,
d) the isolated recombinant polynucleotide comprises a nucleotide sequence encoding the E2A promoter, L4 22K/33K polypeptides and promoter, L4 100 k/hexon assembly polypeptide comprising an N terminal deletion and the E2A DBP, wherein all or part of the L4 100 k/hexon assembly polypeptide is deleted without disruption of the L4 22K/33K start codon,
e) the isolated recombinant polynucleotide comprises a nucleotide sequence encoding the E2A promoter, L4 22K/33K polypeptides and promoter, L4 100 k/hexon assembly polypeptide comprising an N terminal deletion and the E2A DBP, wherein the N-terminal deletion of the L4 100 k/hexon assembly starts at the start codon of L4 100 k/hexon assembly and ends immediately adjacent to the L4 22K/33K promoter,
f) the isolated recombinant polynucleotide comprises a nucleotide sequence having at least 90% identity to SEQ ID NO: 25-34, 56, 57, 106-109, 122-130 or 131,
g) the isolated recombinant polynucleotide comprises a nucleotide sequence having at least 80%, at least 85%, at least 90%, at least 95%, at least 97%, at least 98%, at least 99% or 100% identity to SEQ ID NO: 140-158, 165, 170, 175, 180, 185, 190, 195, 200, 205, 210, 215, 220, 225, 230, 235, 240, 245, 250, 255, or 260, and
h) the isolated recombinant polynucleotide comprises a nucleotide sequence having at least 80%, at least 85%, at least 90%, at least 95%, at least 97%, at least 98%, at least 99% or 100% identity to SEQ ID NO: 265 or 266.

7. The isolated recombinant polynucleotide of claim 1,
a) wherein the AAV rep gene and the AAV cap gene have the same serotype,
b) wherein the AAV rep gene and the AAV cap gene have different serotypes,
c) wherein the AAV rep gene comprises an AAV2 rep gene,
d) wherein the AAV cap gene comprises a serotype selected from the group consisting of AAV8, AAV9, AAV.rh10, AAV.rh20, AAV.rh39, AAV.Rh74, AAV.RHM4-1, AAV.hu32, and AAV.hu37,
e) wherein the AAV cap gene comprises a serotype selected from the group consisting of AAV8 or AAV9 serotype,
f) wherein the AAV cap gene comprises the AAV2, AAV6, AAV8, or AAV9 serotype,
g) wherein the sequence encoding the cap gene comprises one or more mutations that disrupt the expression of the mAAP polypeptide,
h) wherein the sequence encoding the cap gene comprises one or more non-sense mutations in the mAAP ORF that disrupt the expression of the mAAP polypeptide, and/or
i) wherein the sequence encoding the cap gene comprises a mutation in the start codon of the mAAP ORF that disrupt the expression of the mAAP polypeptide.

8. The isolated recombinant polynucleotide of claim 1, wherein
a) the p5 promoter is positioned between about 10 and about 10,000 nucleotides upstream from the AAV rep start codon,
b) the p5 promoter is positioned between about 5,000 and about 10,000 nucleotides upstream from the AAV rep start codon,
c) the p5 promoter is positioned between about 1,000 and about 5,000 nucleotides upstream from the AAV rep start codon,
d) the first, second and/or third nucleotide sequence is positioned between the p5 promoter and the AAV rep start codon upstream from the AAV rep start codon,
e) the first, second and third nucleotide sequences are positioned between the p5 promoter and the AAV rep start codon upstream from the AAV rep start codon, and/or
f) the first, second or third nucleotide sequence is not positioned between the p5 promoter and the AAV rep start codon upstream from the AAV rep start codon.

9. The isolated recombinant polynucleotide of claim 1 comprising
a) a nucleotide sequence having at least 80%, at least 85%, at least 90%, at least 95%, at least 97%, at least 98%, at least 99% or 100% identity to SEQ ID NO: 72-86, 118, 159-161, 168, 173, 178, 183, 188, 193, 198, 203, 208, 213, 218, 223, 228, 233, 238, 243, 248, 253, 558, or 263,
b) a nucleotide sequence having at least 80%, at least 85%, at least 90%, at least 95%, at least 97%, at least 98%, at least 99% or 100% identity to SEQ ID NO: 76 or 77,
c) a nucleotide sequence having at least 80%, at least 85%, at least 90%, at least 95%, at least 97%, at least 98%, at least 99% or 100% identity to SEQ ID NO: 87-101, 119, 162-164, 169, 174, 179, 184, 189, 194, 199, 204, 209, 214, 219, 224, 229, 234, 239, 244, 249, 254, 259, or 264, or
d) a nucleotide sequence having at least 80%, at least 85%, at least 90%, at least 95%, at least 97%, at least 98%, at least 99% or 100% identity to SEQ ID NO: 91 or 92.

10. The isolated recombinant polynucleotide of claim 1, wherein the isolated recombinant polynucleotide is a plasmid comprising a bacterial replication origin and a selectable marker gene.

11. The isolated recombinant polynucleotide of claim 10, wherein
a) the bacterial replication origin or the selectable marker gene are positioned between the fourth nucleotide and the fifth nucleotide such that transcription initiated at the p5 promoter traverses the bacterial replication origin or the selectable marker gene, respectively, before reaching the AAV rep gene, or
b) the bacterial replication origin and the selectable marker gene are positioned between the fourth nucleotide and the fifth nucleotide such that transcription initiated at the p5 promoter traverses the bacterial replication origin and the selectable marker gene before reaching the AAV rep gene.

12. The isolated recombinant polynucleotide of claim 1 comprising a sixth nucleotide sequence encoding a recombinant viral genome comprising at least one AAV inverted terminal repeat (ITR) and a non-AAV nucleic acid sequence encoding a gene product operably linked to sequences which direct expression of the gene product in a target cell.

13. A host cell comprising the isolated recombinant polynucleotide of claim 1, optionally wherein host cell is a HEK293 cell, HEK derived cell, CHO cell, CHO derived cell, HeLa cell, SF-9 cell, BHK cell, Vero cell, or PerC6 cell.

14. A method of producing the isolated recombinant polynucleotide of claim 1 comprising incubating under suitable conditions a host cell comprising the isolated recombinant polynucleotide of claim 1.

15. A method of producing rAAV particles, comprising
a) providing a cell culture comprising a cell;
b) introducing into the cell
i. a polynucleotide of claim 1; and
ii. a polynucleotide comprising a genome comprising at least one AAV inverted terminal repeat (ITR) and a non-AAV nucleic acid sequence encoding a gene product operably linked to sequences which direct expression of the gene product in a target cell, and
c) maintaining the cell culture under conditions that allow production of the rAAV particles,
optionally wherein the introducing of the polynucleotides into the cell is by transfection,
optionally wherein the cell is a HEK293 cell, HEK derived cell, CHO cell, CHO derived cell, HeLa cell, SF-9 cell, BHK cell, Vero cell, or PerC6 cell, and
optionally wherein the gene product is a polypeptide or a double stranded RNA molecule, optionally wherein the gene product is a dystrophin or a microdystrophin.
